SEQUENCE LISTING (60 sequences)

SEQ ID NO: 1 - DNA, 2160 bp, Phomopsis amygdali
atggagttcaaatactcggaagtcgttgaaccctcaacttattacactgaggggctttgcgaaggtatcgatgtgcgcaagagcaagttcaccactcttgaggatcgaggtgccattcgtgctcacgaggactggaacaagcacattggtccttgcggtgaataccgcggaacgcttgggcccagattcagcttcatctcggtggctgtaccggagtgcatacctgagagactggaggtcatctcgtacgcgaacgagtttgcctttctgcacgatgatgttaccgaccatgttggtcacgacacaggcgaagtcgaaaatgatgagatgatgacggttttcctcgaggccgcccataccggtgcgatcgacacctcaaacaaggtcgatattcggcgggcaggaaagaaacggattcaatcacagttattccttgagatgctggcaatcgatcctgaatgtgctaaaaccactatgaaatcttgggcacggttcgtagaggtcgggtccagccgacaacacgagactcgttttgtcgagctggctaagtacataccgtatcgcattatggacgttggagagatgttctggtttggacttgttacctttgggcttggccttcacatacccgatcatgagctcgaactttgccgcgaactaatggcaaatgcctggattgctgtgggcttgcagaacgacatctggtcttggccaaaggagcgagatgccgcgacgctccacggcaaggaccacgtcgttaacgcaatctgggtcctgatgcaggagcatcagacggacgtagatggagctatgcagatctgtcggaagctcatcgtagaatacgtcgccaagtacctcgaggttattgaggctactaagaacgatgagtcgatctcgttagacctgcgcaagtacctcgacgccatgctttacagtatctctgggaatgttgtttggagtcttgaatgcccacgatacaacccagatgtttcattcaacaagacacaattggaatggatgcgtcaaggactgccatctttggagtcatgtcctgtactggcaagaagccctgagatcgactcagacgaatctgcagtttcacccaccgcagatgaatcggactctacagaggatagcttgggaagcggaagtaggcaggattcttcgctgagcactgggttgtctttgtcgcctgttcacagcaacgaaggcaaggatttgcagagagtcgacaccgaccatatattcttcgagaaagcggtcctcgaggcgccctatgactacattgcttccatgccatctaaaggagtccgagatcaatttatcgatgctctgaacgactggttgcgtgttcctgatgtcaaggtgggaaagataaaggatgctgtccgtgttttgcacaactcttcgctgctgctcgacgacttccaagacaactctcccctaagacgcggcaaaccgtcgacgcataacatctttgggtcagcacagactgtgaatacggcgacttactcaataataaaagcaatcggccagatcatggaattttctgcaggcgaatctgtccaagaggtaatgaacagtattatgattttgtttcaaggccaagccatggatctcttctggacatataatggacacgtacccagtgaagaagaatattatcggatgatcgatcaaaaaaccgggcagctgttctcaatcgccaccagtcttcttctaaatgcagcagacaatgagattcccaggacgaaaattcaaagttgtcttcaccggctgacgcgtctacttggacgctgtttccagatacgtgacgattatcagaaccttgtttctgccgactacacaaagcagaagggtttctgcgaggatcttgatgaagggaaatggtctctagcgctgatccacatgattcacaaacagcggagtcatatggcattactcaatgtgctatcaacggggagaaagcatggtggcatgactttggagcagaagcagttcgtgttggacatcatagaggaggagaaaagtctggactataccagatccgtcatgatggacttgcacgttcagctgcgcgctgaaataggacggattgagattctgcttgattctcccaaccctgccatgaggcttttgctggagcttctgcgagtctga

SEQ ID NO: 2 - protein, 719 aa, Phomopsis amygdali
MEFKYSEVVEPSTYYTEGLCEGIDVRKSKFTTLEDRGAIRAHEDWNKHIGPCGEYRGTLGPRFSFISVAVPECIPERLEVISYANEFAFLHDDVTDHVGHDTGEVENDEMMTVFLEAAHTGAIDTSNKVDIRRAGKKRIQSQLFLEMLAIDPECAKTTMKSWARFVEVGSSRQHETRFVELAKYIPYRIMDVGEMFWFGLVTFGLGLHIPDHELELCRELMANAWIAVGLQNDIWSWPKERDAATLHGKDHVVNAIWVLMQEHQTDVDGAMQICRKLIVEYVAKYLEVIEATKNDESISLDLRKYLDAMLYSISGNVVWSLECPRYNPDVSFNKTQLEWMRQGLPSLESCPVLARSPEIDSDESAVSPTADESDSTEDSLGSGSRQDSSLSTGLSLSPVHSNEGKDLQRVDTDHIFFEKAVLEAPYDYIASMPSKGVRDQFIDALNDWLRVPDVKVGKIKDAVRVLHNSSLLLDDFQDNSPLRRGKPSTHNIFGSAQTVNTATYSIIKAIGQIMEFSAGESVQEVMNSIMILFQGQAMDLFWTYNGHVPSEEEYYRMIDQKTGQLFSIATSLLLNAADNEIPRTKIQSCLHRLTRLLGRCFQIRDDYQNLVSADYTKQKGFCEDLDEGKWSLALIHMIHKQRSHMALLNVLSTGRKHGGMTLEQKQFVLDIIEEEKSLDYTRSVMMDLHVQLRAEIGRIEILLDSPNPAMRLLLELLRV

SEQ ID NO: 3 - protein, 12 aa, Artificial Sequence
  Strep tag II
TGSAWSHPQFEK

SEQ ID NO: 4 - DNA, 2157 bp, Artificial Sequence
  Codon optimized sequence
atggaatttaaatattcagaagttgtagaaccatcaacttactatacagaaggattatgtgaaggtattgatgtacgtaaatcaaaatttactactttagaagatcgtggtgctattcgtgcacacgaagactggaacaaacacattggtccatgtggtgaatatcgtggcacattaggtccacgttttagttttatttcagttgcagtacctgaatgcattccagaaagattagaagttatatcttatgctaatgagttcgcttttcttcacgatgatgtaactgaccacgttggtcacgacacaggagaggttgaaaacgatgaaatgatgactgtatttttagaagctgcacatacaggtgctattgacacttctaataaagtagatattcgtcgtgctggtaaaaaacgtattcaatctcaactttttttagaaatgcttgctattgatcctgaatgtgctaaaacaactatgaaaagttgggcacgtttcgtagaggtaggttcaagtcgtcagcacgaaactcgttttgtagaattagcaaaatacattccataccgtattatggatgttggtgaaatgttttggttcggtttagttacttttggtttaggtttacatattcctgatcatgagttagaactttgtagagaacttatggctaatgcttggattgcagtaggtttacaaaatgatatttggagttggccaaaagaacgtgatgctgcaacattacatggtaaagatcatgtagttaatgcaatttgggttttaatgcaagaacaccaaactgacgtagacggtgcaatgcaaatctgccgtaaacttattgtagaatacgtagcaaaatacttagaagtaattgaagctactaaaaatgatgaaagtatttctttagatttacgtaaatatcttgatgcaatgctttacagtattagtggaaacgtagtatggtctttagaatgccctcgttataacccagatgtttcttttaacaaaacacaattagaatggatgcgtcaaggtcttccatctttagagtcttgtcctgtattagctcgttctccagagatagattctgatgaaagtgctgtttcaccaacagctgatgaatcagattctacagaagatagtttaggttctggttcacgtcaagacagttcattatctactggtcttagtttatcaccagttcattctaatgagggaaaagacttacaacgtgttgatactgaccatatttttttcgaaaaagcagtattagaggctccttatgattacatagctagtatgccttctaaaggtgtacgtgatcaattcattgacgctcttaacgattggttacgtgttcctgacgtaaaagttggtaaaatcaaagacgctgttcgtgtacttcataatagttcattattattagatgatttccaagacaattcaccattacgtagaggtaaaccttctactcataacatttttggtagtgcacaaacagttaatacagcaacatactcaatcattaaagctattggacaaataatggaattttctgctggtgaaagtgtacaagaagttatgaactcaattatgattttattccaaggccaagctatggatttattctggacatataatggacatgttccatcagaagaagagtattatcgtatgattgaccaaaaaactggtcaattattctctattgcaacaagtcttcttcttaatgcagctgataatgaaataccacgtactaaaattcaatcatgtcttcaccgtttaacacgtttattaggtcgttgttttcaaattcgtgacgactatcaaaacttagtatctgctgattatactaaacaaaaaggtttttgtgaagaccttgatgagggtaaatggtctttagctttaattcacatgattcacaaacaacgtagtcacatggcattattaaatgttttaagtacaggtcgtaaacatggtggtatgactttagagcaaaaacaattcgtacttgatattattgaagaggaaaaatctttagattatacacgttcagttatgatggacttacacgttcaattacgtgctgaaattggtcgtattgagatccttttagattctcctaatcctgctatgagacttttattagaattattacgtgtt

SEQ ID NO: 5 - DNA, 2196 bp, Artificial Sequence
  Codon optimized sequence
atggaatttaaatattcagaagttgtagaaccatcaacttactatacagaaggattatgtgaaggtattgatgtacgtaaatcaaaatttactactttagaagatcgtggtgctattcgtgcacacgaagactggaacaaacacattggtccatgtggtgaatatcgtggcacattaggtccacgttttagttttatttcagttgcagtacctgaatgcattccagaaagattagaagttatatcttatgctaatgagttcgcttttcttcacgatgatgtaactgaccacgttggtcacgacacaggagaggttgaaaacgatgaaatgatgactgtatttttagaagctgcacatacaggtgctattgacacttctaataaagtagatattcgtcgtgctggtaaaaaacgtattcaatctcaactttttttagaaatgcttgctattgatcctgaatgtgctaaaacaactatgaaaagttgggcacgtttcgtagaggtaggttcaagtcgtcagcacgaaactcgttttgtagaattagcaaaatacattccataccgtattatggatgttggtgaaatgttttggttcggtttagttacttttggtttaggtttacatattcctgatcatgagttagaactttgtagagaacttatggctaatgcttggattgcagtaggtttacaaaatgatatttggagttggccaaaagaacgtgatgctgcaacattacatggtaaagatcatgtagttaatgcaatttgggttttaatgcaagaacaccaaactgacgtagacggtgcaatgcaaatctgccgtaaacttattgtagaatacgtagcaaaatacttagaagtaattgaagctactaaaaatgatgaaagtatttctttagatttacgtaaatatcttgatgcaatgctttacagtattagtggaaacgtagtatggtctttagaatgccctcgttataacccagatgtttcttttaacaaaacacaattagaatggatgcgtcaaggtcttccatctttagagtcttgtcctgtattagctcgttctccagagatagattctgatgaaagtgctgtttcaccaacagctgatgaatcagattctacagaagatagtttaggttctggttcacgtcaagacagttcattatctactggtcttagtttatcaccagttcattctaatgagggaaaagacttacaacgtgttgatactgaccatatttttttcgaaaaagcagtattagaggctccttatgattacatagctagtatgccttctaaaggtgtacgtgatcaattcattgacgctcttaacgattggttacgtgttcctgacgtaaaagttggtaaaatcaaagacgctgttcgtgtacttcataatagttcattattattagatgatttccaagacaattcaccattacgtagaggtaaaccttctactcataacatttttggtagtgcacaaacagttaatacagcaacatactcaatcattaaagctattggacaaataatggaattttctgctggtgaaagtgtacaagaagttatgaactcaattatgattttattccaaggccaagctatggatttattctggacatataatggacatgttccatcagaagaagagtattatcgtatgattgaccaaaaaactggtcaattattctctattgcaacaagtcttcttcttaatgcagctgataatgaaataccacgtactaaaattcaatcatgtcttcaccgtttaacacgtttattaggtcgttgttttcaaattcgtgacgactatcaaaacttagtatctgctgattatactaaacaaaaaggtttttgtgaagaccttgatgagggtaaatggtctttagctttaattcacatgattcacaaacaacgtagtcacatggcattattaaatgttttaagtacaggtcgtaaacatggtggtatgactttagagcaaaaacaattcgtacttgatattattgaagaggaaaaatctttagattatacacgttcagttatgatggacttacacgttcaattacgtgctgaaattggtcgtattgagatccttttagattctcctaatcctgctatgagacttttattagaattattacgtgttaccggtagtgcttggtcacaccctcaatttgagaaataa

SEQ ID NO: 6 - protein, 731 aa, Phomopsis amygdali
  MISC_FEATURE (720)..(731): Strep tag II
MEFKYSEVVEPSTYYTEGLCEGIDVRKSKFTTLEDRGAIRAHEDWNKHIGPCGEYRGTLGPRFSFISVAVPECIPERLEVISYANEFAFLHDDVTDHVGHDTGEVENDEMMTVFLEAAHTGAIDTSNKVDIRRAGKKRIQSQLFLEMLAIDPECAKTTMKSWARFVEVGSSRQHETRFVELAKYIPYRIMDVGEMFWFGLVTFGLGLHIPDHELELCRELMANAWIAVGLQNDIWSWPKERDAATLHGKDHVVNAIWVLMQEHQTDVDGAMQICRKLIVEYVAKYLEVIEATKNDESISLDLRKYLDAMLYSISGNVVWSLECPRYNPDVSFNKTQLEWMRQGLPSLESCPVLARSPEIDSDESAVSPTADESDSTEDSLGSGSRQDSSLSTGLSLSPVHSNEGKDLQRVDTDHIFFEKAVLEAPYDYIASMPSKGVRDQFIDALNDWLRVPDVKVGKIKDAVRVLHNSSLLLDDFQDNSPLRRGKPSTHNIFGSAQTVNTATYSIIKAIGQIMEFSAGESVQEVMNSIMILFQGQAMDLFWTYNGHVPSEEEYYRMIDQKTGQLFSIATSLLLNAADNEIPRTKIQSCLHRLTRLLGRCFQIRDDYQNLVSADYTKQKGFCEDLDEGKWSLALIHMIHKQRSHMALLNVLSTGRKHGGMTLEQKQFVLDIIEEEKSLDYTRSVMMDLHVQLRAEIGRIEILLDSPNPAMRLLLELLRVTGSAWSHPQFEK

SEQ ID NO: 7 - DNA, 2157 bp, Artificial Sequence
  Codon optimized seqeunce
atggaatttaaatattcagaagttgttgaaccatcaacatattatacagaaggtttatgtgaaggtattgatgttcgtaaatcaaaatttacaacattagaagatcgtggtgctattcgtgctcatgaagattggaataaacatattggtccatgtggtgaatatcgtggtacattaggtccacgtttttcatttatttcagttgctgttccagaatgtattccagaacgtttagaagttatttcatacgctaatgaatttgcttttttacatgatgatgttacagatcatgttggtcatgatacaggtgaagttgaaaatgatgaaatgatgacagtttttttagaagctgctcatacaggtgctattgatacatcaaataaagttgatattcgtcgtgctggtaaaaaacgtattcaatcacaattatttttagaaatgttagctattgatccagaatgtgctaaaacaacaatgaaatcatgggctcgttttgttgaagttggttcatcacgtcaacatgaaacacgttttgttgaattagctaaatatattccatatcgtattatggatgttggtgaaatgttttggtttggtttagttacatttggtttaggtttacatattccagatcatgaattagaattatgtcgtgaacttatggctaatgcttggattgctgttggtttacaaaatgatatttggtcatggccaaaagaacgtgatgctgctacattacatggtaaagatcatgttgttaatgctatttgggttttaatgcaagaacatcaaacagatgttgatggtgctatgcaaatttgtcgtaaacttattgttgaatatgttgctaaatatttagaagttattgaagctacaaaaaatgatgaatcaatttcattagatttacgtaaatatttagatgctatgttatattcaatttcaggtaatgttgtttggtcattagaatgtccacgttataatccagatgtttcatttaataaaacacaattagaatggatgcgtcaaggtttaccatcattagaatcatgtccagttttagctcgttcaccagaaattgattcagatgaatcagcagtttcaccaactgctgatgaatcagattcaacagaagattcattaggttcaggttcacgtcaagattcatcattatcaacaggtttatcattatcaccagttcattcaaatgaaggtaaagatttacaacgtgttgatacagatcatattttttttgaaaaagctgttttagaagctccatacgattatattgcttcaatgccatcaaaaggtgttcgtgaccaatttattgatgctttaaatgattggttacgtgttccagatgttaaagttggtaaaattaaagatgctgttcgtgttttacataattcatcattattattagatgattttcaagataattcaccattacgtcgtggtaaaccatcaacacataatatttttggttcagctcaaacagttaatacagctacatattcaattattaaagctattggtcaaattatggaattttctgctggtgagtcagttcaagaagttatgaactcaattatgattttatttcaaggtcaagctatggatttattttggacatataatggtcatgttccatcagaagaagaatattatcgtatgattgaccaaaaaacaggtcaattattttcaattgctacatcattattattaaatgctgctgataatgaaattccacgtacaaaaattcaatcatgtttacatcgtttaacacgtttattaggtcgttgttttcaaattcgtgatgattatcaaaatttagtttctgctgattacactaaacaaaaaggattctgtgaagatttagatgaaggtaaatggtcattagctttaattcacatgattcataaacaacgttcacacatggctttattaaatgttttatcaacaggtcgtaaacatggtggtatgacattagaacaaaaacaatttgttttagatattattgaagaagaaaaatcattagattatacacgttcagttatgatggatcttcatgttcaattacgtgctgaaattggtcgtattgaaattttattagattcaccaaatccagctatgcgtttattattagaattattacgtgtt

SEQ ID NO: 8 - DNA, 2196 bp, Artificial Sequence
  Codon optimized sequence
atggaatttaaatattcagaagttgttgaaccatcaacatattatacagaaggtttatgtgaaggtattgatgttcgtaaatcaaaatttacaacattagaagatcgtggtgctattcgtgctcatgaagattggaataaacatattggtccatgtggtgaatatcgtggtacattaggtccacgtttttcatttatttcagttgctgttccagaatgtattccagaacgtttagaagttatttcatacgctaatgaatttgcttttttacatgatgatgttacagatcatgttggtcatgatacaggtgaagttgaaaatgatgaaatgatgacagtttttttagaagctgctcatacaggtgctattgatacatcaaataaagttgatattcgtcgtgctggtaaaaaacgtattcaatcacaattatttttagaaatgttagctattgatccagaatgtgctaaaacaacaatgaaatcatgggctcgttttgttgaagttggttcatcacgtcaacatgaaacacgttttgttgaattagctaaatatattccatatcgtattatggatgttggtgaaatgttttggtttggtttagttacatttggtttaggtttacatattccagatcatgaattagaattatgtcgtgaacttatggctaatgcttggattgctgttggtttacaaaatgatatttggtcatggccaaaagaacgtgatgctgctacattacatggtaaagatcatgttgttaatgctatttgggttttaatgcaagaacatcaaacagatgttgatggtgctatgcaaatttgtcgtaaacttattgttgaatatgttgctaaatatttagaagttattgaagctacaaaaaatgatgaatcaatttcattagatttacgtaaatatttagatgctatgttatattcaatttcaggtaatgttgtttggtcattagaatgtccacgttataatccagatgtttcatttaataaaacacaattagaatggatgcgtcaaggtttaccatcattagaatcatgtccagttttagctcgttcaccagaaattgattcagatgaatcagcagtttcaccaactgctgatgaatcagattcaacagaagattcattaggttcaggttcacgtcaagattcatcattatcaacaggtttatcattatcaccagttcattcaaatgaaggtaaagatttacaacgtgttgatacagatcatattttttttgaaaaagctgttttagaagctccatacgattatattgcttcaatgccatcaaaaggtgttcgtgaccaatttattgatgctttaaatgattggttacgtgttccagatgttaaagttggtaaaattaaagatgctgttcgtgttttacataattcatcattattattagatgattttcaagataattcaccattacgtcgtggtaaaccatcaacacataatatttttggttcagctcaaacagttaatacagctacatattcaattattaaagctattggtcaaattatggaattttctgctggtgagtcagttcaagaagttatgaactcaattatgattttatttcaaggtcaagctatggatttattttggacatataatggtcatgttccatcagaagaagaatattatcgtatgattgaccaaaaaacaggtcaattattttcaattgctacatcattattattaaatgctgctgataatgaaattccacgtacaaaaattcaatcatgtttacatcgtttaacacgtttattaggtcgttgttttcaaattcgtgatgattatcaaaatttagtttctgctgattacactaaacaaaaaggattctgtgaagatttagatgaaggtaaatggtcattagctttaattcacatgattcataaacaacgttcacacatggctttattaaatgttttatcaacaggtcgtaaacatggtggtatgacattagaacaaaaacaatttgttttagatattattgaagaagaaaaatcattagattatacacgttcagttatgatggatcttcatgttcaattacgtgctgaaattggtcgtattgaaattttattagattcaccaaatccagctatgcgtttattattagaattattacgtgttaccggtagtgcttggtcacaccctcaatttgagaaataa

SEQ ID NO: 9 - DNA, 2841 bp, Phaeosphaeria nodorum
atgtttgccaaattcgatatgcttgaagaagaagcccgggcccttgttcgaaaagtaggtaacgcagttgatccgatttacggcttcagtaccacgagctgtcagatctacgacacagcctgggcggccatgatatctaaagaagagcatggagacaaagtgtggctctttcccgagagtttcaaatatctccttgaaaagcaaggcgaggacggtagctgggaaagacatcccaggtcgaagacggttggcgtcttgaacacagcggctgcgtgtcttgcactcttgcgtcatgtcaaaaaccctctacagctacaagatatcgctgctcaagatatcgaattgcgcatccagcgtgggctaagatcacttgaagaacaacttatcgcctgggacgacgtgttggacaccaatcacattggtgttgagatgattgtccccgcattattggactatttgcaggcagaagacgaaaacgtggactttgaattcgagagccacagcctactgatgcagatgtacaaggaaaaaatggcccgcttcagtcctgagtctctctaccgggcgcggccatcgtcagccctccacaatctggaggctctgattggcaagctggatttcgacaaggttggacatcacctgtacaatggttcaatgatggcatctccgtcctctacagcagcttttttgatgcatgcttccccatggagtcacgaggctgaagcatatttgcggcatgtattcgaagctggtacaggcaaaggttcgggcggatttccaggcacatatcctactacgtactttgagttgaactgggtgctgtctactcttatgaaaagcgggtttactctatctgatctggagtgtgatgagctttccagcatcgcaaacaccattgctgaagggttcgagtgtgatcatggtgtgatcggttttgctccacgtgcagtggatgttgacgacacggccaaagggctactgacgctgactttgcttggcatggatgaaggtgtcagtcctgcgccaatgattgccatgttcgaagccaaagatcatttcttgacgtttctgggggagagggacccaagtttcacgtcgaactgtcacgtgctgctttctctgttgcatcgaacggatctactgcaatacctgcctcagatacggaaaacgacgacgttcctgtgcgaagcatggtgggcgtgcgatgggcagatcaaagacaagtggcatctgagccatctgtacccaacaatgttgatggtgcaagcgtttgcggaaattttgctcaagagcgccgagggagagcctctccacgacgctttcgacgcggccacgctatcgcgagtctccatctgcgtgttccaggcgtgcttacgaacgctgctggcccagagccaggatggatcgtggcatggccaaccagaggcttcgtgctatgcggttctaacgctcgccgagtcgggtcggctcgtgttgctgcaggccctgcagccgcagattgcagctgccatggaaaaggccgcagacgtcatgcaggccggacgctggagctgcagcgaccatgactgtgactggacgtccaaaacggcatatcgcgtggaccttgttgctgcagcgtaccgcctagccgccatgaaggctagctccaacttaaccttcaccgtcgacgacaatgtgtcgaagcgtagcaacggtttccagcagctggtcggccggacagatctgttctctggggtaccggcatgggaattgcaggcgtcatttcttgagagcgctctatttgttcccctgctcagaaaccaccggctcgacgtatttgaccgagacgatatcaaggtcagcaaggatcattatctcgacatgattcccttcacttgggtcggctgcaataaccggtcacgcacatatgtttcgacatcgtttctatttgacatgatgatcatctccatgctgggataccagattgacgagttcttcgaagctgaggccgcccccgcgtttgcccagtgcatcggccaactccaccaggtggttgataaagtcgttgatgaagtgattgatgaagtcgttgataaagtcgttggtaaagtcgtcggtaaagtcgtcggtaaagtcgttgatgagcgagtcgactcaccaacgcacgaagccattgcaatttgcaacatcgaggcttcgctgcggcggttcgtcgaccatgtgctgcatcaccagcatgtacttcacgccagccagcaggagcaagacatcctgtggcgcgagctgcgggcttttttgcacgctcatgttgtccagatggccgacaactccaccttagcgccacccggtcgcaccttcttcgactgggttcgcactaccgctgcagatcacgtggcatgtgcctactcgtttgcatttgcatgctgcatcacctctgccaccatcggccagggtcagagcatgtttgccacggtcaacgaactatacctcgtgcaagccgctgcccgccatatgacaacaatgtgccgcatgtgtaacgacattggctctgtcgaccgcgatttcatcgaagctaacattaactcggtccatttcccagaattctcaaccttgagcttggttgccgacaagaaaaaggctcttgcacgcctggctgcgtatgagaagtcttgtctgacccatacactcgaccagttcgagaacgaggttcttcaatctcccagagtctcctcggctgcgtctggtgatttccgcacaagaaaggtggccgttgtacgcttttttgctgatgtcacggatttttacgaccagctatacatactccgcgacctctccagctctttgaaacacgtcggcacgtag

SEQ ID NO: 10 - protein, 946 aa, Phaeosphaeria nodorum
MFAKFDMLEEEARALVRKVGNAVDPIYGFSTTSCQIYDTAWAAMISKEEHGDKVWLFPESFKYLLEKQGEDGSWERHPRSKTVGVLNTAAACLALLRHVKNPLQLQDIAAQDIELRIQRGLRSLEEQLIAWDDVLDTNHIGVEMIVPALLDYLQAEDENVDFEFESHSLLMQMYKEKMARFSPESLYRARPSSALHNLEALIGKLDFDKVGHHLYNGSMMASPSSTAAFLMHASPWSHEAEAYLRHVFEAGTGKGSGGFPGTYPTTYFELNWVLSTLMKSGFTLSDLECDELSSIANTIAEGFECDHGVIGFAPRAVDVDDTAKGLLTLTLLGMDEGVSPAPMIAMFEAKDHFLTFLGERDPSFTSNCHVLLSLLHRTDLLQYLPQIRKTTTFLCEAWWACDGQIKDKWHLSHLYPTMLMVQAFAEILLKSAEGEPLHDAFDAATLSRVSICVFQACLRTLLAQSQDGSWHGQPEASCYAVLTLAESGRLVLLQALQPQIAAAMEKAADVMQAGRWSCSDHDCDWTSKTAYRVDLVAAAYRLAAMKASSNLTFTVDDNVSKRSNGFQQLVGRTDLFSGVPAWELQASFLESALFVPLLRNHRLDVFDRDDIKVSKDHYLDMIPFTWVGCNNRSRTYVSTSFLFDMMIISMLGYQIDEFFEAEAAPAFAQCIGQLHQVVDKVVDEVIDEVVDKVVGKVVGKVVGKVVDERVDSPTHEAIAICNIEASLRRFVDHVLHHQHVLHASQQEQDILWRELRAFLHAHVVQMADNSTLAPPGRTFFDWVRTTAADHVACAYSFAFACCITSATIGQGQSMFATVNELYLVQAAARHMTTMCRMCNDIGSVDRDFIEANINSVHFPEFSTLSLVADKKKALARLAAYEKSCLTHTLDQFENEVLQSPRVSSAASGDFRTRKVAVVRFFADVTDFYDQLYILRDLSSSLKHVGT

SEQ ID NO: 11 - DNA, 2835 bp, Artificial Sequence
  Codon optimized sequence
tttgctaaatttgatatgttagaagaagaagctcgtgctttagttcgtaaagttggtaatgctgttgatccaatttatggtttttcaacaacatcatgtcaaatttatgatacagcttgggctgctatgatttcaaaagaagaacatggtgataaagtttggttatttccagaatcatttaaatatttattagaaaaacaaggtgaagatggttcatgggaacgtcatccacgttcaaaaacagttggtgttttaaatactgctgctgcttgtttagctttattacgtcatgttaaaaatccattacaattacaagatattgctgctcaagatattgaattacgtattcaacgtggtttacgttcattagaagaacaacttattgcttgggatgatgttttagatacaaatcatattggtgttgaaatgattgttccagctttattagattatttacaagctgaagatgaaaatgttgattttgaatttgaatcacattcattacttatgcaaatgtataaagaaaaaatggctcgtttttcaccagaatcattatatcgtgctcgtccatcatcagctttacataatttagaagctcttattggtaaattagattttgataaagttggtcatcatttatataatggttcaatgatggcttcaccatcatcaacagcagcttttttaatgcacgcttcaccttggtcacatgaagctgaggcttatttacgtcatgtttttgaagctggtacaggtaaaggttcaggtggttttccaggtacatatccaacaacatattttgaattaaattgggttttatcaacacttatgaaatcaggttttacattatcagatttagaatgtgatgaattatcatcaattgctaatacaattgctgaaggttttgaatgtgatcatggtgttattggttttgctccacgtgctgttgatgttgatgatacagctaaaggtttattaacattaacattattaggtatggatgaaggtgtttcaccagctccaatgattgctatgtttgaagctaaagatcattttttaacatttttaggtgaacgtgatccatcatttacatcaaattgtcatgttttattatcattattacatcgtacagatttattacaatatttaccacaaattcgtaaaacaacaacatttttatgtgaggcttggtgggcttgtgatggtcaaattaaagataaatggcatttatcacatttatatccaacaatgttaatggttcaggcttttgctgaaattttattaaaatctgctgaaggtgaaccattacatgatgcttttgatgctgctacattatcacgtgtttcaatttgtgtttttcaggcttgtttacgtacattattagctcaatcacaagatggttcatggcatggtcaaccagaggcttcatgttatgctgttttaacattagctgaatcaggtcgtttagttttattacaagcattacaaccacaaattgctgctgctatggaaaaagctgctgatgttatgcaagctggtcgttggtcatgttcagatcatgattgtgattggacatcaaaaacagcttatcgtgttgatttagttgctgctgcttatcgtttagctgctatgaaagcatcatcaaatttaacatttacagttgatgataatgtttcaaaacgttcaaatggttttcaacaattagttggtcgtacagatttattttcaggtgttccagcttgggaattacaagcatcatttttagaatcagctttatttgttccattattacgtaatcatcgtttagatgtttttgatcgtgatgatattaaagtttcaaaagatcattatttagatatgattccatttacatgggttggttgtaataatcgttcacgtacatacgtttcaacatcatttttatttgatatgatgattatttcaatgttaggttatcaaattgatgaattttttgaagctgaagctgctccagcttttgctcaatgtattggtcaattacatcaagttgttgataaagttgttgatgaagttattgatgaagttgtagataaagttgttggtaaagttgtaggtaaagttgttggtaaagttgttgatgaacgtgttgattcaccaacacatgaagctattgctatttgtaatattgaagcatcattacgtcgttttgttgatcatgttttacatcatcaacatgttttacatgcttcacaacaagaacaagatattttatggcgtgaattacgtgcttttttacatgctcatgttgttcaaatggctgataattcaacattagctccaccaggtcgtacattttttgattgggttcgtacaactgctgctgatcatgttgcttgtgcttattcatttgcttttgcttgttgtattacatcagctacaattggtcaaggtcaatcaatgtttgctacagttaatgaattatatttagttcaagctgctgctcgtcacatgacaacaatgtgtcgtatgtgtaatgatattggttcagttgatcgtgattttattgaagctaatattaactcagttcattttccagaattttcaacattatcattagttgctgataaaaaaaaagcattagctcgtttagctgcttatgaaaaatcatgtttaacacatacattagatcaatttgaaaatgaagttttacaatcaccacgtgtttcatcagcagcttcaggtgattttcgtacacgtaaagttgctgttgttcgtttttttgctgatgttacagatttttatgatcaattatatattttacgtgatttatcatcatcattaaaacatgttggtaca

SEQ ID NO: 12 - protein, 10 aa, Artificial Sequence
  Tag
MDYKDDDDKG

SEQ ID NO: 13 - DNA, 2874 bp, Artificial Sequence
  Codon optimized sequence
atggattataaagatgacgatgacaaaggttttgctaaatttgatatgttagaagaagaagctcgtgctttagttcgtaaagttggtaatgctgttgatccaatttatggtttttcaacaacatcatgtcaaatttatgatacagcttgggctgctatgatttcaaaagaagaacatggtgataaagtttggttatttccagaatcatttaaatatttattagaaaaacaaggtgaagatggttcatgggaacgtcatccacgttcaaaaacagttggtgttttaaatactgctgctgcttgtttagctttattacgtcatgttaaaaatccattacaattacaagatattgctgctcaagatattgaattacgtattcaacgtggtttacgttcattagaagaacaacttattgcttgggatgatgttttagatacaaatcatattggtgttgaaatgattgttccagctttattagattatttacaagctgaagatgaaaatgttgattttgaatttgaatcacattcattacttatgcaaatgtataaagaaaaaatggctcgtttttcaccagaatcattatatcgtgctcgtccatcatcagctttacataatttagaagctcttattggtaaattagattttgataaagttggtcatcatttatataatggttcaatgatggcttcaccatcatcaacagcagcttttttaatgcacgcttcaccttggtcacatgaagctgaggcttatttacgtcatgtttttgaagctggtacaggtaaaggttcaggtggttttccaggtacatatccaacaacatattttgaattaaattgggttttatcaacacttatgaaatcaggttttacattatcagatttagaatgtgatgaattatcatcaattgctaatacaattgctgaaggttttgaatgtgatcatggtgttattggttttgctccacgtgctgttgatgttgatgatacagctaaaggtttattaacattaacattattaggtatggatgaaggtgtttcaccagctccaatgattgctatgtttgaagctaaagatcattttttaacatttttaggtgaacgtgatccatcatttacatcaaattgtcatgttttattatcattattacatcgtacagatttattacaatatttaccacaaattcgtaaaacaacaacatttttatgtgaggcttggtgggcttgtgatggtcaaattaaagataaatggcatttatcacatttatatccaacaatgttaatggttcaggcttttgctgaaattttattaaaatctgctgaaggtgaaccattacatgatgcttttgatgctgctacattatcacgtgtttcaatttgtgtttttcaggcttgtttacgtacattattagctcaatcacaagatggttcatggcatggtcaaccagaggcttcatgttatgctgttttaacattagctgaatcaggtcgtttagttttattacaagcattacaaccacaaattgctgctgctatggaaaaagctgctgatgttatgcaagctggtcgttggtcatgttcagatcatgattgtgattggacatcaaaaacagcttatcgtgttgatttagttgctgctgcttatcgtttagctgctatgaaagcatcatcaaatttaacatttacagttgatgataatgtttcaaaacgttcaaatggttttcaacaattagttggtcgtacagatttattttcaggtgttccagcttgggaattacaagcatcatttttagaatcagctttatttgttccattattacgtaatcatcgtttagatgtttttgatcgtgatgatattaaagtttcaaaagatcattatttagatatgattccatttacatgggttggttgtaataatcgttcacgtacatacgtttcaacatcatttttatttgatatgatgattatttcaatgttaggttatcaaattgatgaattttttgaagctgaagctgctccagcttttgctcaatgtattggtcaattacatcaagttgttgataaagttgttgatgaagttattgatgaagttgtagataaagttgttggtaaagttgtaggtaaagttgttggtaaagttgttgatgaacgtgttgattcaccaacacatgaagctattgctatttgtaatattgaagcatcattacgtcgttttgttgatcatgttttacatcatcaacatgttttacatgcttcacaacaagaacaagatattttatggcgtgaattacgtgcttttttacatgctcatgttgttcaaatggctgataattcaacattagctccaccaggtcgtacattttttgattgggttcgtacaactgctgctgatcatgttgcttgtgcttattcatttgcttttgcttgttgtattacatcagctacaattggtcaaggtcaatcaatgtttgctacagttaatgaattatatttagttcaagctgctgctcgtcacatgacaacaatgtgtcgtatgtgtaatgatattggttcagttgatcgtgattttattgaagctaatattaactcagttcattttccagaattttcaacattatcattagttgctgataaaaaaaaagcattagctcgtttagctgcttatgaaaaatcatgtttaacacatacattagatcaatttgaaaatgaagttttacaatcaccacgtgtttcatcagcagcttcaggtgattttcgtacacgtaaagttgctgttgttcgtttttttgctgatgttacagatttttatgatcaattatatattttacgtgatttatcatcatcattaaaacatgttggtacaaccggttaa

SEQ ID NO: 14 - protein, 955 aa, Phaeosphaeria nodorum
  MISC_FEATURE (1)..(10): Tag
MDYKDDDDKGFAKFDMLEEEARALVRKVGNAVDPIYGFSTTSCQIYDTAWAAMISKEEHGDKVWLFPESFKYLLEKQGEDGSWERHPRSKTVGVLNTAAACLALLRHVKNPLQLQDIAAQDIELRIQRGLRSLEEQLIAWDDVLDTNHIGVEMIVPALLDYLQAEDENVDFEFESHSLLMQMYKEKMARFSPESLYRARPSSALHNLEALIGKLDFDKVGHHLYNGSMMASPSSTAAFLMHASPWSHEAEAYLRHVFEAGTGKGSGGFPGTYPTTYFELNWVLSTLMKSGFTLSDLECDELSSIANTIAEGFECDHGVIGFAPRAVDVDDTAKGLLTLTLLGMDEGVSPAPMIAMFEAKDHFLTFLGERDPSFTSNCHVLLSLLHRTDLLQYLPQIRKTTTFLCEAWWACDGQIKDKWHLSHLYPTMLMVQAFAEILLKSAEGEPLHDAFDAATLSRVSICVFQACLRTLLAQSQDGSWHGQPEASCYAVLTLAESGRLVLLQALQPQIAAAMEKAADVMQAGRWSCSDHDCDWTSKTAYRVDLVAAAYRLAAMKASSNLTFTVDDNVSKRSNGFQQLVGRTDLFSGVPAWELQASFLESALFVPLLRNHRLDVFDRDDIKVSKDHYLDMIPFTWVGCNNRSRTYVSTSFLFDMMIISMLGYQIDEFFEAEAAPAFAQCIGQLHQVVDKVVDEVIDEVVDKVVGKVVGKVVGKVVDERVDSPTHEAIAICNIEASLRRFVDHVLHHQHVLHASQQEQDILWRELRAFLHAHVVQMADNSTLAPPGRTFFDWVRTTAADHVACAYSFAFACCITSATIGQGQSMFATVNELYLVQAAARHMTTMCRMCNDIGSVDRDFIEANINSVHFPEFSTLSLVADKKKALARLAAYEKSCLTHTLDQFENEVLQSPRVSSAASGDFRTRKVAVVRFFADVTDFYDQLYILRDLSSSLKHVGT

SEQ ID NO: 15 - DNA, 1806 bp, Ricinus communis
atggcattgccatcagctgctatgcaatccaaccctgaaaagcttaacttatttcacagattgtcaagcttacccaccactagcttggaatatggcaataatcgcttccctttcttttcctcatctgccaagtcacactttaaaaaaccaactcaagcatgtttatcctcaacaacccaccaagaagttcgtccattagcatactttcctcctactgtctggggcaatcgctttgcttccttgaccttcaatccatcggaatttgaatcgtatgatgaacgggtaattgtgctgaagaaaaaagttaaggacatattaatttcatctacaagtgattcagtggagaccgttattttaatcgacttattatgtcggcttggcgtatcatatcactttgaaaatgatattgaagagctactaagtaaaatcttcaactcccagcctgaccttgtcgatgaaaaagaatgtgatctctacactgcggcaattgtattccgagttttcagacagcatggttttaaaatgtcttcggatgtgtttagcaaattcaaggacagtgatggtaagttcaaggaatccctacggggtgatgctaagggtatgctcagcctttttgaagcttcccatctaagtgtgcatggagaagacattcttgaagaagcctttgctttcaccaaggattacttacagtcctctgcagttgagttattccctaatctcaaaaggcatataacgaacgccctagagcagcctttccacagtggcgtgccgaggctagaggccaggaaattcatcgatctatacgaagctgatattgaatgccggaatgaaactctgctcgagtttgcaaagttggattataatagagttcagttattgcaccaacaagagctgtgccagttctcaaagtggtggaaagacctgaatcttgcttcggatattccttatgcaagagacagaatggcagagattttcttttgggcagtcgcgatgtactttgagcctgactatgcacacacccgaatgattattgcgaaggttgtattgcttatatcactaatagatgatacaattgatgcgtatgcaacaatggaggaaactcatattcttgctgaagcagtcgcaaggtgggacatgagctgcctcgagaagctgccagattacatgaaagttatttataaactattgctaaacaccttctctgaattcgagaaagaattgacggcggaaggcaagtcctacagcgtcaaatacggaagggaagcgtttcaagaactagtgagaggttactacctggaggctgtatggcgcgacgagggtaaaataccatcgttcgatgactacttgtataatggatccatgaccaccggattgcctctcgtctcaacagcttctttcatgggagttcaagaaattacaggtctcaacgaattccaatggctggaaactaatcccaaattaagttatgcttccggtgcattcatccgacttgtcaacgacttaacttctcatgtgactgaacaacaaagaggacacgttgcatcttgcatcgactgctatatgaaccaacatggagtttccaaagacgaagcagtcaaaatacttcaaaaaatggctacagattgttggaaagaaattaatgaagaatgtatgaggcagagtcaagtgtcagtgggtcacctaatgagaatagttaatctggcacgtcttacggatgtgagttacaagtatggagacggttacactgattcccagcaattgaaacaatttgttaagggattgttcgttgatccaatttctatttga

SEQ ID NO: 16 - protein, 601 aa, Ricinus communis
MALPSAAMQSNPEKLNLFHRLSSLPTTSLEYGNNRFPFFSSSAKSHFKKPTQACLSSTTHQEVRPLAYFPPTVWGNRFASLTFNPSEFESYDERVIVLKKKVKDILISSTSDSVETVILIDLLCRLGVSYHFENDIEELLSKIFNSQPDLVDEKECDLYTAAIVFRVFRQHGFKMSSDVFSKFKDSDGKFKESLRGDAKGMLSLFEASHLSVHGEDILEEAFAFTKDYLQSSAVELFPNLKRHITNALEQPFHSGVPRLEARKFIDLYEADIECRNETLLEFAKLDYNRVQLLHQQELCQFSKWWKDLNLASDIPYARDRMAEIFFWAVAMYFEPDYAHTRMIIAKVVLLISLIDDTIDAYATMEETHILAEAVARWDMSCLEKLPDYMKVIYKLLLNTFSEFEKELTAEGKSYSVKYGREAFQELVRGYYLEAVWRDEGKIPSFDDYLYNGSMTTGLPLVSTASFMGVQEITGLNEFQWLETNPKLSYASGAFIRLVNDLTSHVTEQQRGHVASCIDCYMNQHGVSKDEAVKILQKMATDCWKEINEECMRQSQVSVGHLMRIVNLARLTDVSYKYGDGYTDSQQLKQFVKGLFVDPISI

SEQ ID NO: 17 - DNA, 1638 bp, Artificial Sequence
  Codon optimized sequence
atgtcaacaacacatcaagaagttcgtccattagcttattttccaccaacagtttggggtaatcgttttgcttcattaacatttaatccatcagaatttgaatcttatgatgaacgtgttattgttttaaaaaaaaaagttaaagatattttaatttcatcaacatcagattcagttgaaacagttattttaattgatttattatgtcgtttaggtgtttcatatcattttgaaaatgatattgaagaattattatcaaaaatttttaattcacaaccagatttagttgatgaaaaagaatgtgatttatatacagcagctattgtttttcgtgtttttcgtcaacatggttttaaaatgtcatcagatgttttttcaaaatttaaagattcagatggtaaatttaaagaatcattacgtggtgatgctaaaggtatgttatcattatttgaagcatcacatttatcagttcatggtgaagatattttagaagaagcatttgcttttacaaaagattatttacaatcatctgctgttgaattatttccaaatttaaaacgtcatattacaaatgctttagaacaaccatttcattcaggtgttccacgtttagaagctcgtaaatttattgatttatatgaagctgatattgaatgtcgtaatgaaacattattagaatttgctaaattagattataatcgtgttcaattattacatcaacaagaattatgtcaattttcaaaatggtggaaagatttaaatttagcttcagatattccttatgctcgtgatcgtatggctgaaatttttttttgggctgttgctatgtattttgaaccagattatgctcatacacgtatgattattgctaaagttgttttacttatttctttaattgatgatacaattgatgcttatgctacaatggaagaaacacatattttagctgaagctgttgctcgttgggatatgtcatgtttagaaaaattaccagattatatgaaagttatttataaattattattaaatacattttcagaatttgaaaaagaattaacagcagaaggtaaatcatattcagttaaatatggtcgtgaagcatttcaagaattagttcgtggttattatttagaagctgtttggcgtgatgaaggtaaaattccatcatttgatgattatttatataatggttcaatgacaacaggtttaccattagtttcaacagcttcatttatgggtgttcaagaaattacaggtttaaatgaatttcaatggttagaaacaaatccaaaattatcttatgcttcaggtgcttttattcgtttagttaatgatttaacatctcatgttacagaacaacaacgtggtcatgttgcttcatgtattgattgttatatgaatcaacatggtgtttcaaaagatgaagctgttaaaattttacaaaaaatggctacagattgttggaaagaaatcaatgaagaatgtatgcgtcaatcacaagtttcagttggtcatttaatgcgtattgttaatttagctcgtttaacagatgtttcatataaatatggtgatggttatacagattcacaacaattaaaacaatttgttaaaggtttatttgttgatccaatttcaatt

SEQ ID NO: 18 - DNA, 1683 bp, Artificial Sequence
  Codon optimized sequence
atgtcaacaacacatcaagaagttcgtccattagcttattttccaccaacagtttggggtaatcgttttgcttcattaacatttaatccatcagaatttgaatcttatgatgaacgtgttattgttttaaaaaaaaaagttaaagatattttaatttcatcaacatcagattcagttgaaacagttattttaattgatttattatgtcgtttaggtgtttcatatcattttgaaaatgatattgaagaattattatcaaaaatttttaattcacaaccagatttagttgatgaaaaagaatgtgatttatatacagcagctattgtttttcgtgtttttcgtcaacatggttttaaaatgtcatcagatgttttttcaaaatttaaagattcagatggtaaatttaaagaatcattacgtggtgatgctaaaggtatgttatcattatttgaagcatcacatttatcagttcatggtgaagatattttagaagaagcatttgcttttacaaaagattatttacaatcatctgctgttgaattatttccaaatttaaaacgtcatattacaaatgctttagaacaaccatttcattcaggtgttccacgtttagaagctcgtaaatttattgatttatatgaagctgatattgaatgtcgtaatgaaacattattagaatttgctaaattagattataatcgtgttcaattattacatcaacaagaattatgtcaattttcaaaatggtggaaagatttaaatttagcttcagatattccttatgctcgtgatcgtatggctgaaatttttttttgggctgttgctatgtattttgaaccagattatgctcatacacgtatgattattgctaaagttgttttacttatttctttaattgatgatacaattgatgcttatgctacaatggaagaaacacatattttagctgaagctgttgctcgttgggatatgtcatgtttagaaaaattaccagattatatgaaagttatttataaattattattaaatacattttcagaatttgaaaaagaattaacagcagaaggtaaatcatattcagttaaatatggtcgtgaagcatttcaagaattagttcgtggttattatttagaagctgtttggcgtgatgaaggtaaaattccatcatttgatgattatttatataatggttcaatgacaacaggtttaccattagtttcaacagcttcatttatgggtgttcaagaaattacaggtttaaatgaatttcaatggttagaaacaaatccaaaattatcttatgcttcaggtgcttttattcgtttagttaatgatttaacatctcatgttacagaacaacaacgtggtcatgttgcttcatgtattgattgttatatgaatcaacatggtgtttcaaaagatgaagctgttaaaattttacaaaaaatggctacagattgttggaaagaaatcaatgaagaatgtatgcgtcaatcacaagtttcagttggtcatttaatgcgtattgttaatttagctcgtttaacagatgtttcatataaatatggtgatggttatacagattcacaacaattaaaacaatttgttaaaggtttatttgttgatccaatttcaattaccggtattaattcagcttggtcacatccacaatttgaaaaataa

SEQ ID NO: 19 - protein, 14 aa, Artificial Sequence
  Strep tag
TGINSAWSHPQFEK

SEQ ID NO: 20 - protein, 560 aa, Ricinus communis
  MISC_FEATURE (547)..(560): Strep tag
MSTTHQEVRPLAYFPPTVWGNRFASLTFNPSEFESYDERVIVLKKKVKDILISSTSDSVETVILIDLLCRLGVSYHFENDIEELLSKIFNSQPDLVDEKECDLYTAAIVFRVFRQHGFKMSSDVFSKFKDSDGKFKESLRGDAKGMLSLFEASHLSVHGEDILEEAFAFTKDYLQSSAVELFPNLKRHITNALEQPFHSGVPRLEARKFIDLYEADIECRNETLLEFAKLDYNRVQLLHQQELCQFSKWWKDLNLASDIPYARDRMAEIFFWAVAMYFEPDYAHTRMIIAKVVLLISLIDDTIDAYATMEETHILAEAVARWDMSCLEKLPDYMKVIYKLLLNTFSEFEKELTAEGKSYSVKYGREAFQELVRGYYLEAVWRDEGKIPSFDDYLYNGSMTTGLPLVSTASFMGVQEITGLNEFQWLETNPKLSYASGAFIRLVNDLTSHVTEQQRGHVASCIDCYMNQHGVSKDEAVKILQKMATDCWKEINEECMRQSQVSVGHLMRIVNLARLTDVSYKYGDGYTDSQQLKQFVKGLFVDPISITGINSAWSHPQFEK

SEQ ID NO: 21 - DNA, 2793 bp, Artificial Sequence
  Codon optimized sequence
atgtcaacaacacatcaagaagttcgtccattagcttattttccaccaacagtttggggtaatcgttttgcaagtttaacatttaatccatcagaatttgaatcatacgatgaacgtgttattgttttaaaaaaaaaagttaaagatattttaatttcatcaacatcagattcagttgaaacagttattttaattgatttattatgtcgtttaggtgtttcatatcattttgaaaatgatattgaagaattattatcaaaaatttttaattcacaaccagatttagttgatgaaaaagaatgtgatttatatacagcagctattgtttttcgtgtttttcgtcaacatggttttaaaatgtcatcagatgttttttcaaaatttaaagattcagatggtaaatttaaagaatcattacgtggtgatgctaaaggtatgttatcattatttgaagcatcacatttatcagttcatggtgaagatattttagaagaagcatttgcttttacaaaagattatttacaatcatctgctgttgaattatttccaaatttaaaacgtcatattacaaatgctttagaacaaccatttcattcaggtgttccacgtttagaagctcgtaaatttattgatttatatgaagctgatattgaatgtcgtaatgaaacattattagaatttgctaaattagattataatcgtgttcaattattacatcaacaagaattatgtcaattttcaaaatggtggaaagatttaaatttagcttcagatattccatacgctcgtgatcgtatggctgaaatttttttttgggctgttgctatgtattttgaaccagattatgctcatacacgtatgattattgctaaagttgttcttttaatttctttaattgatgatacaattgatgcttatgctacaatggaagaaacacatattttagctgaagctgttgctcgttgggatatgtcatgtttagaaaaattaccagattatatgaaagttatttataaattattattaaatacattttcagaatttgaaaaagaattaactgctgaaggtaaatcatattcagttaaatatggtcgtgaagcatttcaagaattagttcgtggttattatttagaagctgtttggcgtgatgaaggtaaaattccatcatttgatgattatttatataatggttcaatgacaacaggtttaccattagtttcaacagcttcatttatgggtgttcaagaaattacaggtttaaatgaatttcaatggttagaaacaaatccaaaattatcatacgcttcaggtgcttttattcgtttagttaatgatttaacatcacatgttacagaacaacaacgtggtcatgttgcttcatgtattgattgttatatgaatcaacatggtgtttcaaaagatgaagctgttaaaattttacaaaaaatggctactgattgttggaaagaaattaacgaagaatgtatgcgtcaatcacaagtttcagttggtcatttaatgcgtattgttaatttagctcgtttaacagatgtttcatataaatatggtgatggttatacagattcacaacaattaaaacaatttgttaaaggtttatttgttgatccaatttcaattacacaattagaatggatgcgtcaaggtttaccatcattagaatcatgtccagttttagctcgttcaccagaaattgattcagatgaatcagcagtttcaccaacagcagatgaatcagattcaacagaagattcattaggttcaggttcacgtcaagattcatcattatcaacaggtttatcattatcaccagttcattcaaatgaaggtaaagatttacaacgtgttgatacagatcatattttttttgaaaaagctgttttagaagctccatacgattatattgcttcaatgccatcaaaaggtgttcgtgatcaatttattgatgctttaaatgattggttacgtgttccagatgttaaagttggtaaaattaaagatgctgttcgtgttttacataattcatcattattattagatgattttcaagataattcaccattacgtcgtggtaaaccatcaacacataatatttttggttcagctcaaacagttaatacagctacatattcaattattaaagctattggtcaaattatggaattttctgctggtgaatcagttcaagaagttatgaactcaattatgattttatttcaaggtcaagctatggatttattttggacatataatggtcatgttccatcagaagaagaatattatcgtatgattgatcaaaaaacaggtcaattattttcaattgctacatcattattattaaatgctgctgataatgaaattccacgtacaaaaattcaatcatgtttacatcgtttaacacgtttattaggtcgttgttttcaaattcgtgatgattatcaaaatttagtttcagcagattatacaaaacaaaaaggtttttgtgaagatttagatgaaggtaaatggtcattagctttaattcacatgattcataaacaacgttcacacatggctttattaaatgttttatcaacaggtcgtaaacatggtggtatgacattagaacaaaaacaatttgttttagatattattgaagaagaaaaatcattagattatacacgttcagttatgatggatttacatgttcaattacgtgctgaaattggtcgtattgaaattttattagattcaccaaatccagctatgcgtttattattagaattattacgtgtt

SEQ ID NO: 22 - protein, 931 aa, Artificial Sequence
  Fusion protein
MSTTHQEVRPLAYFPPTVWGNRFASLTFNPSEFESYDERVIVLKKKVKDILISSTSDSVETVILIDLLCRLGVSYHFENDIEELLSKIFNSQPDLVDEKECDLYTAAIVFRVFRQHGFKMSSDVFSKFKDSDGKFKESLRGDAKGMLSLFEASHLSVHGEDILEEAFAFTKDYLQSSAVELFPNLKRHITNALEQPFHSGVPRLEARKFIDLYEADIECRNETLLEFAKLDYNRVQLLHQQELCQFSKWWKDLNLASDIPYARDRMAEIFFWAVAMYFEPDYAHTRMIIAKVVLLISLIDDTIDAYATMEETHILAEAVARWDMSCLEKLPDYMKVIYKLLLNTFSEFEKELTAEGKSYSVKYGREAFQELVRGYYLEAVWRDEGKIPSFDDYLYNGSMTTGLPLVSTASFMGVQEITGLNEFQWLETNPKLSYASGAFIRLVNDLTSHVTEQQRGHVASCIDCYMNQHGVSKDEAVKILQKMATDCWKEINEECMRQSQVSVGHLMRIVNLARLTDVSYKYGDGYTDSQQLKQFVKGLFVDPISITQLEWMRQGLPSLESCPVLARSPEIDSDESAVSPTADESDSTEDSLGSGSRQDSSLSTGLSLSPVHSNEGKDLQRVDTDHIFFEKAVLEAPYDYIASMPSKGVRDQFIDALNDWLRVPDVKVGKIKDAVRVLHNSSLLLDDFQDNSPLRRGKPSTHNIFGSAQTVNTATYSIIKAIGQIMEFSAGESVQEVMNSIMILFQGQAMDLFWTYNGHVPSEEEYYRMIDQKTGQLFSIATSLLLNAADNEIPRTKIQSCLHRLTRLLGRCFQIRDDYQNLVSADYTKQKGFCEDLDEGKWSLALIHMIHKQRSHMALLNVLSTGRKHGGMTLEQKQFVLDIIEEEKSLDYTRSVMMDLHVQLRAEIGRIEILLDSPNPAMRLLLELLRV

SEQ ID NO: 23 - protein, 191 aa, Artificial Sequence
  CLIP-8xhis tag
TGDKDCEMKRTTLDSPLGKLELSGCEQGLHEIIFLGKGTSAADAVEVPAPAAVLGGPEPLIQATAWLNAYFHQPEAIEEFPVPALHHPVFQQESFTRQVLWKLLKVVKFGEVISESHLAALVGNPAATAAVNTALDGNPVPILIPCHRVVQGDSDVGPYLGGLAVKEWLLAHEGHRLGKPGLGHHHHHHHH

SEQ ID NO: 24 - DNA, 3369 bp, Artificial sequence
  Fusion protein
atgtcaacaacacatcaagaagttcgtccattagcttattttccaccaacagtttggggtaatcgttttgcaagtttaacatttaatccatcagaatttgaatcatacgatgaacgtgttattgttttaaaaaaaaaagttaaagatattttaatttcatcaacatcagattcagttgaaacagttattttaattgatttattatgtcgtttaggtgtttcatatcattttgaaaatgatattgaagaattattatcaaaaatttttaattcacaaccagatttagttgatgaaaaagaatgtgatttatatacagcagctattgtttttcgtgtttttcgtcaacatggttttaaaatgtcatcagatgttttttcaaaatttaaagattcagatggtaaatttaaagaatcattacgtggtgatgctaaaggtatgttatcattatttgaagcatcacatttatcagttcatggtgaagatattttagaagaagcatttgcttttacaaaagattatttacaatcatctgctgttgaattatttccaaatttaaaacgtcatattacaaatgctttagaacaaccatttcattcaggtgttccacgtttagaagctcgtaaatttattgatttatatgaagctgatattgaatgtcgtaatgaaacattattagaatttgctaaattagattataatcgtgttcaattattacatcaacaagaattatgtcaattttcaaaatggtggaaagatttaaatttagcttcagatattccatacgctcgtgatcgtatggctgaaatttttttttgggctgttgctatgtattttgaaccagattatgctcatacacgtatgattattgctaaagttgttcttttaatttctttaattgatgatacaattgatgcttatgctacaatggaagaaacacatattttagctgaagctgttgctcgttgggatatgtcatgtttagaaaaattaccagattatatgaaagttatttataaattattattaaatacattttcagaatttgaaaaagaattaactgctgaaggtaaatcatattcagttaaatatggtcgtgaagcatttcaagaattagttcgtggttattatttagaagctgtttggcgtgatgaaggtaaaattccatcatttgatgattatttatataatggttcaatgacaacaggtttaccattagtttcaacagcttcatttatgggtgttcaagaaattacaggtttaaatgaatttcaatggttagaaacaaatccaaaattatcatacgcttcaggtgcttttattcgtttagttaatgatttaacatcacatgttacagaacaacaacgtggtcatgttgcttcatgtattgattgttatatgaatcaacatggtgtttcaaaagatgaagctgttaaaattttacaaaaaatggctactgattgttggaaagaaattaacgaagaatgtatgcgtcaatcacaagtttcagttggtcatttaatgcgtattgttaatttagctcgtttaacagatgtttcatataaatatggtgatggttatacagattcacaacaattaaaacaatttgttaaaggtttatttgttgatccaatttcaattacacaattagaatggatgcgtcaaggtttaccatcattagaatcatgtccagttttagctcgttcaccagaaattgattcagatgaatcagcagtttcaccaacagcagatgaatcagattcaacagaagattcattaggttcaggttcacgtcaagattcatcattatcaacaggtttatcattatcaccagttcattcaaatgaaggtaaagatttacaacgtgttgatacagatcatattttttttgaaaaagctgttttagaagctccatacgattatattgcttcaatgccatcaaaaggtgttcgtgatcaatttattgatgctttaaatgattggttacgtgttccagatgttaaagttggtaaaattaaagatgctgttcgtgttttacataattcatcattattattagatgattttcaagataattcaccattacgtcgtggtaaaccatcaacacataatatttttggttcagctcaaacagttaatacagctacatattcaattattaaagctattggtcaaattatggaattttctgctggtgaatcagttcaagaagttatgaactcaattatgattttatttcaaggtcaagctatggatttattttggacatataatggtcatgttccatcagaagaagaatattatcgtatgattgatcaaaaaacaggtcaattattttcaattgctacatcattattattaaatgctgctgataatgaaattccacgtacaaaaattcaatcatgtttacatcgtttaacacgtttattaggtcgttgttttcaaattcgtgatgattatcaaaatttagtttcagcagattatacaaaacaaaaaggtttttgtgaagatttagatgaaggtaaatggtcattagctttaattcacatgattcataaacaacgttcacacatggctttattaaatgttttatcaacaggtcgtaaacatggtggtatgacattagaacaaaaacaatttgttttagatattattgaagaagaaaaatcattagattatacacgttcagttatgatggatttacatgttcaattacgtgctgaaattggtcgtattgaaattttattagattcaccaaatccagctatgcgtttattattagaattattacgtgttaccggtgataaagattgtgaaatgaaacgtacaacattagattcaccattaggtaaattagaattatcaggttgtgaacaaggtttacatgaaattatttttttaggtaaaggtacatctgctgcagatgctgttgaagttccagctcctgctgcagttttaggtggtccagaacctttaattcaagctacagcttggttaaatgcttattttcatcaaccagaagctattgaagaatttccagttccagctttacatcatccagtttttcaacaagaatcatttacacgtcaagtattatggaaattattaaaagttgttaaatttggtgaagttatttcagaatcacatttagctgctttagttggtaatccagcagctacagcagcagttaatacagctttagatggtaatccagttccaattttaattccatgtcatcgtgttgttcaaggtgattcagatgttggtccatatttaggtggtttagctgttaaagaatggttattagctcatgaaggtcatcgtttaggtaaaccaggtttaggtcatcaccatcatcaccatcaccactaa

SEQ ID NO: 25 - protein, 1122 aa, Artificial Sequence
  Fusion protein
MSTTHQEVRPLAYFPPTVWGNRFASLTFNPSEFESYDERVIVLKKKVKDILISSTSDSVETVILIDLLCRLGVSYHFENDIEELLSKIFNSQPDLVDEKECDLYTAAIVFRVFRQHGFKMSSDVFSKFKDSDGKFKESLRGDAKGMLSLFEASHLSVHGEDILEEAFAFTKDYLQSSAVELFPNLKRHITNALEQPFHSGVPRLEARKFIDLYEADIECRNETLLEFAKLDYNRVQLLHQQELCQFSKWWKDLNLASDIPYARDRMAEIFFWAVAMYFEPDYAHTRMIIAKVVLLISLIDDTIDAYATMEETHILAEAVARWDMSCLEKLPDYMKVIYKLLLNTFSEFEKELTAEGKSYSVKYGREAFQELVRGYYLEAVWRDEGKIPSFDDYLYNGSMTTGLPLVSTASFMGVQEITGLNEFQWLETNPKLSYASGAFIRLVNDLTSHVTEQQRGHVASCIDCYMNQHGVSKDEAVKILQKMATDCWKEINEECMRQSQVSVGHLMRIVNLARLTDVSYKYGDGYTDSQQLKQFVKGLFVDPISITQLEWMRQGLPSLESCPVLARSPEIDSDESAVSPTADESDSTEDSLGSGSRQDSSLSTGLSLSPVHSNEGKDLQRVDTDHIFFEKAVLEAPYDYIASMPSKGVRDQFIDALNDWLRVPDVKVGKIKDAVRVLHNSSLLLDDFQDNSPLRRGKPSTHNIFGSAQTVNTATYSIIKAIGQIMEFSAGESVQEVMNSIMILFQGQAMDLFWTYNGHVPSEEEYYRMIDQKTGQLFSIATSLLLNAADNEIPRTKIQSCLHRLTRLLGRCFQIRDDYQNLVSADYTKQKGFCEDLDEGKWSLALIHMIHKQRSHMALLNVLSTGRKHGGMTLEQKQFVLDIIEEEKSLDYTRSVMMDLHVQLRAEIGRIEILLDSPNPAMRLLLELLRVTGDKDCEMKRTTLDSPLGKLELSGCEQGLHEIIFLGKGTSAADAVEVPAPAAVLGGPEPLIQATAWLNAYFHQPEAIEEFPVPALHHPVFQQESFTRQVLWKLLKVVKFGEVISESHLAALVGNPAATAAVNTALDGNPVPILIPCHRVVQGDSDVGPYLGGLAVKEWLLAHEGHRLGKPGLGHHHHHHHH

SEQ ID NO: 26 - DNA, 2607 bp, Abies grandis
atggccatgccttcctcttcattgtcatcacagattcccactgctgctcatcatctaactgctaacgcacaatccattccgcatttctccacgacgctgaatgctggaagcagtgctagcaaacggagaagcttgtacctacgatggggtaaaggttcaaacaagatcattgcctgtgttggagaaggtggtgcaacctctgttccttatcagtctgctgaaaagaatgattcgctttcttcttctacattggtgaaacgagaatttcctccaggattttggaaggatgatcttatcgattctctaacgtcatctcacaaggttgcagcatcagacgagaagcgtatcgagacattaatatccgagattaagaatatgtttagatgtatgggctatggcgaaacgaatccctctgcatatgacactgcttgggtagcaaggattccagcagttgatggctctgacaaccctcactttcctgagacggttgaatggattcttcaaaatcagttgaaagatgggtcttggggtgaaggattctacttcttggcatatgacagaatactggctacacttgcatgtattattacccttaccctctggcgtactggggagacacaagtacagaaaggtattgaattcttcaggacacaagctggaaagatggaagatgaagctgatagtcataggccaagtggatttgaaatagtatttcctgcaatgctaaaggaagctaaaatcttaggcttggatctgccttacgatttgccattcctgaaacaaatcatcgaaaagcgggaggctaagcttaaaaggattcccactgatgttctctatgcccttccaacaacgttattgtattctttggaaggtttacaagaaatagtagactggcagaaaataatgaaacttcaatccaaggatggatcatttctcagctctccggcatctacagcggctgtattcatgcgtacagggaacaaaaagtgcttggatttcttgaactttgtcttgaagaaattcggaaaccatgtgccttgtcactatccgcttgatctatttgaacgtttgtgggcggttgatacagttgagcggctaggtatcgatcgtcatttcaaagaggagatcaaggaagcattggattatgtttacagccattgggacgaaagaggcattggatgggcgagagagaatcctgttcctgatattgatgatacagccatgggccttcgaatcttgagattacatggatacaatgtatcctcagatgttttaaaaacatttagagatgagaatggggagttcttttgcttcttgggtcaaacacagagaggagttacagacatgttaaacgtcaatcgttgttcacatgtttcatttccgggagaaacgatcatggaagaagcaaaactctgtaccgaaaggtatctgaggaatgctctggaaaatgtggatgcctttgacaaatgggcttttaaaaagaatattcggggagaggtagagtatgcactcaaatatccctggcataagagtatgccaaggttggaggctagaagctatattgaaaactatgggccagatgatgtgtggcttggaaaaactgtatatatgatgccatacatttcgaatgaaaagtatttagaactagcgaaactggacttcaataaggtgcagtctatacaccaaacagagcttcaagatcttcgaaggtggtggaaatcatccggtttcacggatctgaatttcactcgtgagcgtgtgacggaaatatatttctcaccggcatcctttatctttgagcccgagttttctaagtgcagagaggtttatacaaaaacttccaatttcactgttattttagatgatctttatgacgcccatggatctttagacgatcttaagttgttcacagaatcagtcaaaagatgggatctatcactagtggaccaaatgccacaacaaatgaaaatatgttttgtgggtttctacaatacttttaatgatatagcaaaagaaggacgtgagaggcaagggcgcgatgtgctaggctacattcaaaatgtttggaaagtccaacttgaagcttacacgaaagaagcagaatggtctgaagctaaatatgtgccatccttcaatgaatacatagagaatgcgagtgtgtcaatagcattgggaacagtcgttctcattagtgctcttttcactggggaggttcttacagatgaagtactctccaaaattgatcgcgaatctagatttcttcaactcatgggcttaacagggcgtttggtgaatgacaccaaaacttatcaggcagagagaggtcaaggtgaggtggcttctgccatacaatgttatatgaaggaccatcctaaaatctctgaagaagaagctctacaacatgtctatagtgtcatggaaaatgccctcgaagagttgaatagggagtttgtgaataacaaaataccggatatttacaaaagactggtttttgaaactgcaagaataatgcaactcttttatatgcaaggggatggtttgacactatcacatgatatggaaattaaagagcatgtcaaaaattgcctcttccaaccagttgcctag

SEQ ID NO: 27 - protein, 868 aa, Abies grandis
MAMPSSSLSSQIPTAAHHLTANAQSIPHFSTTLNAGSSASKRRSLYLRWGKGSNKIIACVGEGGATSVPYQSAEKNDSLSSSTLVKREFPPGFWKDDLIDSLTSSHKVAASDEKRIETLISEIKNMFRCMGYGETNPSAYDTAWVARIPAVDGSDNPHFPETVEWILQNQLKDGSWGEGFYFLAYDRILATLACIITLTLWRTGETQVQKGIEFFRTQAGKMEDEADSHRPSGFEIVFPAMLKEAKILGLDLPYDLPFLKQIIEKREAKLKRIPTDVLYALPTTLLYSLEGLQEIVDWQKIMKLQSKDGSFLSSPASTAAVFMRTGNKKCLDFLNFVLKKFGNHVPCHYPLDLFERLWAVDTVERLGIDRHFKEEIKEALDYVYSHWDERGIGWARENPVPDIDDTAMGLRILRLHGYNVSSDVLKTFRDENGEFFCFLGQTQRGVTDMLNVNRCSHVSFPGETIMEEAKLCTERYLRNALENVDAFDKWAFKKNIRGEVEYALKYPWHKSMPRLEARSYIENYGPDDVWLGKTVYMMPYISNEKYLELAKLDFNKVQSIHQTELQDLRRWWKSSGFTDLNFTRERVTEIYFSPASFIFEPEFSKCREVYTKTSNFTVILDDLYDAHGSLDDLKLFTESVKRWDLSLVDQMPQQMKICFVGFYNTFNDIAKEGRERQGRDVLGYIQNVWKVQLEAYTKEAEWSEAKYVPSFNEYIENASVSIALGTVVLISALFTGEVLTDEVLSKIDRESRFLQLMGLTGRLVNDTKTYQAERGQGEVASAIQCYMKDHPKISEEEALQHVYSVMENALEELNREFVNNKIPDIYKRLVFETARIMQLFYMQGDGLTLSHDMEIKEHVKNCLFQPVA

SEQ ID NO: 28 - DNA, 2397 bp, Artificial Sequence
  Codon optimized seqeunce
caatctgctgaaaagaacgactctttatcaagttctacattagttaagagagaatttccacccggtttctggaaagacgacttaatcgacagtttaacttcaagtcacaaagtagctgctagcgatgaaaaacgtatcgaaaccttaatttcagaaattaagaatatgtttcgttgtatgggttatggtgagacaaatccatcagcttatgatactgcttgggtagctcgcatcccagcagttgatggatcagataatcctcactttccagagactgtggaatggatcttacaaaatcaattaaaagatggttcttggggtgaaggtttttacttccttgcttatgatcgcattttagccactttagcttgtattatcacacttacactttggcgtactggagaaacacaagtacagaaaggtatcgaatttttccgcactcaagcaggtaaaatggaagatgaagcagattcacaccgtccaagtggttttgagattgtatttcctgctatgttaaaagaggctaagattttaggcttagatttaccttatgatcttccttttcttaaacaaattattgaaaagagagaagctaagttaaaacgtattcctacagatgttttatatgctttaccaactactttactttattcattagaaggtttacaagaaatagtagactggcaaaaaatcatgaaattacaaagtaaagatggtagtttcttatcttctcctgcctcaacagcagcagtatttatgagaacaggtaacaaaaagtgtttagatttcttaaatttcgtgcttaaaaagttcggtaatcatgttccatgccactatcctttagacctttttgagcgtctttgggcagttgatactgttgaaagattaggtattgaccgtcattttaaagaagaaataaaagaggctttagactatgtgtattcacactgggacgaacgtggtattggttgggctcgtgaaaaccccgttccagatattgacgatacagcaatgggtcttcgtattttacgtcttcatggttacaatgttagcagcgatgttcttaaaacatttcgtgatgaaaatggtgagttcttttgctttttaggacaaacacaaagaggtgtgactgatatgttaaatgttaatcgttgtagccatgtatctttccctggtgaaactataatggaagaggcaaaattatgtactgaacgttacttacgcaacgcattagaaaatgtagacgcttttgataagtgggcatttaagaaaaacattcgtggtgaggtagaatatgctcttaaatatccttggcataaatcaatgccacgtttagaagcacgttcatatattgaaaattacggtccagatgatgtttggttaggtaaaactgtttatatgatgccttacatttcaaatgaaaagtacttagagttagctaaacttgattttaacaaagttcagtcaatccaccagacagaacttcaagacttacgccgttggtggaaaagttctggttttacagatttaaactttacaagagaacgtgttactgaaatttacttttcacctgcatcttttatcttcgaaccagaatttagtaaatgtcgtgaggtttatacaaaaacttctaattttactgtaattttagacgatttatatgacgctcatggctctttagatgacttaaaactttttacagagagtgttaaacgttgggatttatctttagttgaccaaatgccccagcagatgaaaatctgttttgtaggtttctataatacattcaacgatattgctaaagaaggtagagaacgtcaaggtcgtgatgttttaggttatattcaaaacgtatggaaagtacaacttgaagcatatactaaagaagcagaatggtcagaagcaaaatatgttcctagttttaacgaatacattgaaaatgcttcagtttcaattgccttaggtacagtagtacttatcagtgctttatttaccggagaagttttaacagatgaagttttatctaaaattgaccgtgaaagtagattcttacagttaatgggcttaactggacgtttagtaaatgatactaaaacatatcaagctgagcgtggtcaaggtgaagttgctagtgcaattcaatgttatatgaaagaccaccctaaaattagtgaagaagaagcattacaacatgtatattctgtaatggaaaatgcattagaagaattaaatcgtgagttcgttaacaacaaaattccagacatctataaacgtcttgttttcgaaactgcacgtataatgcaattattttacatgcaaggtgatggtttaacattaagtcacgatatggaaattaaagagcacgtaaagaattgtttattccagccagtagctggt

SEQ ID NO: 29 - protein, 26 aa, Artificial Sequence
  TEV-FLAG tag
TGENLYFQGSGGGGSDYKDDDDKGTG

SEQ ID NO: 30 - DNA, 2487 bp, Artificial Sequence
  Codon optimized sequence
atggtaccacaatctgctgaaaagaacgactctttatcaagttctacattagttaagagagaatttccacccggtttctggaaagacgacttaatcgacagtttaacttcaagtcacaaagtagctgctagcgatgaaaaacgtatcgaaaccttaatttcagaaattaagaatatgtttcgttgtatgggttatggtgagacaaatccatcagcttatgatactgcttgggtagctcgcatcccagcagttgatggatcagataatcctcactttccagagactgtggaatggatcttacaaaatcaattaaaagatggttcttggggtgaaggtttttacttccttgcttatgatcgcattttagccactttagcttgtattatcacacttacactttggcgtactggagaaacacaagtacagaaaggtatcgaatttttccgcactcaagcaggtaaaatggaagatgaagcagattcacaccgtccaagtggttttgagattgtatttcctgctatgttaaaagaggctaagattttaggcttagatttaccttatgatcttccttttcttaaacaaattattgaaaagagagaagctaagttaaaacgtattcctacagatgttttatatgctttaccaactactttactttattcattagaaggtttacaagaaatagtagactggcaaaaaatcatgaaattacaaagtaaagatggtagtttcttatcttctcctgcctcaacagcagcagtatttatgagaacaggtaacaaaaagtgtttagatttcttaaatttcgtgcttaaaaagttcggtaatcatgttccatgccactatcctttagacctttttgagcgtctttgggcagttgatactgttgaaagattaggtattgaccgtcattttaaagaagaaataaaagaggctttagactatgtgtattcacactgggacgaacgtggtattggttgggctcgtgaaaaccccgttccagatattgacgatacagcaatgggtcttcgtattttacgtcttcatggttacaatgttagcagcgatgttcttaaaacatttcgtgatgaaaatggtgagttcttttgctttttaggacaaacacaaagaggtgtgactgatatgttaaatgttaatcgttgtagccatgtatctttccctggtgaaactataatggaagaggcaaaattatgtactgaacgttacttacgcaacgcattagaaaatgtagacgcttttgataagtgggcatttaagaaaaacattcgtggtgaggtagaatatgctcttaaatatccttggcataaatcaatgccacgtttagaagcacgttcatatattgaaaattacggtccagatgatgtttggttaggtaaaactgtttatatgatgccttacatttcaaatgaaaagtacttagagttagctaaacttgattttaacaaagttcagtcaatccaccagacagaacttcaagacttacgccgttggtggaaaagttctggttttacagatttaaactttacaagagaacgtgttactgaaatttacttttcacctgcatcttttatcttcgaaccagaatttagtaaatgtcgtgaggtttatacaaaaacttctaattttactgtaattttagacgatttatatgacgctcatggctctttagatgacttaaaactttttacagagagtgttaaacgttgggatttatctttagttgaccaaatgccccagcagatgaaaatctgttttgtaggtttctataatacattcaacgatattgctaaagaaggtagagaacgtcaaggtcgtgatgttttaggttatattcaaaacgtatggaaagtacaacttgaagcatatactaaagaagcagaatggtcagaagcaaaatatgttcctagttttaacgaatacattgaaaatgcttcagtttcaattgccttaggtacagtagtacttatcagtgctttatttaccggagaagttttaacagatgaagttttatctaaaattgaccgtgaaagtagattcttacagttaatgggcttaactggacgtttagtaaatgatactaaaacatatcaagctgagcgtggtcaaggtgaagttgctagtgcaattcaatgttatatgaaagaccaccctaaaattagtgaagaagaagcattacaacatgtatattctgtaatggaaaatgcattagaagaattaaatcgtgagttcgttaacaacaaaattccagacatctataaacgtcttgttttcgaaactgcacgtataatgcaattattttacatgcaaggtgatggtttaacattaagtcacgatatggaaattaaagagcacgtaaagaattgtttattccagccagtagctggtaccggtgaaaacttatactttcaaggctcaggtggcggtggaagtgattacaaagatgatgatgataaaggaaccggttaa

SEQ ID NO: 31 - protein, 828 aa, Abies grandis
  MISC_FEATURE (803)..(828): TEV-FLAG tag
MVPQSAEKNDSLSSSTLVKREFPPGFWKDDLIDSLTSSHKVAASDEKRIETLISEIKNMFRCMGYGETNPSAYDTAWVARIPAVDGSDNPHFPETVEWILQNQLKDGSWGEGFYFLAYDRILATLACIITLTLWRTGETQVQKGIEFFRTQAGKMEDEADSHRPSGFEIVFPAMLKEAKILGLDLPYDLPFLKQIIEKREAKLKRIPTDVLYALPTTLLYSLEGLQEIVDWQKIMKLQSKDGSFLSSPASTAAVFMRTGNKKCLDFLNFVLKKFGNHVPCHYPLDLFERLWAVDTVERLGIDRHFKEEIKEALDYVYSHWDERGIGWARENPVPDIDDTAMGLRILRLHGYNVSSDVLKTFRDENGEFFCFLGQTQRGVTDMLNVNRCSHVSFPGETIMEEAKLCTERYLRNALENVDAFDKWAFKKNIRGEVEYALKYPWHKSMPRLEARSYIENYGPDDVWLGKTVYMMPYISNEKYLELAKLDFNKVQSIHQTELQDLRRWWKSSGFTDLNFTRERVTEIYFSPASFIFEPEFSKCREVYTKTSNFTVILDDLYDAHGSLDDLKLFTESVKRWDLSLVDQMPQQMKICFVGFYNTFNDIAKEGRERQGRDVLGYIQNVWKVQLEAYTKEAEWSEAKYVPSFNEYIENASVSIALGTVVLISALFTGEVLTDEVLSKIDRESRFLQLMGLTGRLVNDTKTYQAERGQGEVASAIQCYMKDHPKISEEEALQHVYSVMENALEELNREFVNNKIPDIYKRLVFETARIMQLFYMQGDGLTLSHDMEIKEHVKNCLFQPVAGTGENLYFQGSGGGGSDYKDDDDKGTG

SEQ ID NO: 32 - DNA, 2589 bp, Taxus brevifolia
atggctcagctctcatttaatgcagcgctgaagatgaacgcattggggaacaaggcaatccacgatccaacgaattgcagagccaaatctgagcgccaaatgatgtgggtttgctccagatcagggcgaaccagagtaaaaatgtcgagaggaagtggtggtcctggtcctgtcgtaatgatgagcagcagcactggcactagcaaggtggtttccgagacttccagtaccattgtggatgatatccctcgactctccgccaattatcatggcgatctgtggcaccacaatgttatacaaactctggagacaccgtttcgtgagagttctacttaccaagaacgggcagatgagctggttgtgaaaattaaagatatgttcaatgcgctcggagacggagatatcagtccgtctgcatacgacactgcgtgggtggcgaggctggcgaccatttcctctgatggatctgagaagccacggtttcctcaggccctcaactgggttttcaacaaccagctccaggatggatcgtggggtatcgaatcgcactttagtttatgcgatcgattgcttaacacgaccaattctgttatcgccctctcggtttggaaaacagggcacagccaagtacaacaaggtgctgagtttattgcagagaatctaagattactcaatgaggaagatgagttgtccccggatttccaaataatctttcctgctctgctgcaaaaggcaaaagcgttggggatcaatcttccttacgatcttccatttatcaaatatttgtcgacaacacgggaagccaggcttacagatgtttctgcggcagcagacaatattccagccaacatgttgaatgcgttggaaggtctcgaggaagttattgactggaacaagattatgaggtttcaaagtaaagatggatctttcctgagctcccctgcctccactgcctgtgtactgatgaatacaggggacgaaaaatgtttcacttttctcaacaatctgctcgacaaattcggcggctgcgtgccctgtatgtattccatcgatctgctggaacgcctttcgctggttgataacattgagcatctcggaatcggtcgccatttcaaacaagaaatcaaaggagctcttgattatgtctacagacattggagtgaaaggggcatcggttggggcagagacagccttgttccagatctcaacaccacagccctcggcctgcgaactcttcgcatgcacggatacaatgtttcttcagacgttttgaataatttcaaagatgaaaacgggcggttcttctcctctgcgggccaaacccatgtcgaattgagaagcgtggtgaatcttttcagagcttccgaccttgcatttcctgacgaaagagctatggacgatgctagaaaatttgcagaaccatatcttagagaggcacttgcaacgaaaatctcaaccaatacaaaactattcaaagagattgagtacgtggtggagtacccttggcacatgagtatcccacgcttagaagccagaagttatattgattcatatgacgacaattatgtatggcagaggaagactctatatagaatgccatctttgagtaattcaaaatgtttagaattggcaaaattggacttcaatatcgtacaatctttgcatcaagaggagttgaagcttctaacaagatggtggaaggaatccggcatggcagatataaatttcactcgacaccgagtggcggaggtttatttttcatcagctacatttgaacccgaatattctgccactagaattgccttcacaaaaattggttgtttacaagtcctttttgatgatatggctgacatctttgcaacactagatgaattgaaaagtttcactgagggagtaaagagatgggatacatctttgctacatgagattccagagtgtatgcaaacttgctttaaagtttggttcaaattaatggaagaagtaaataatgatgtggttaaggtacaaggacgtgacatgctcgctcacataagaaaaccctgggagttgtacttcaattgttatgtacaagaaagggagtggcttgaagccgggtatataccaacttttgaagagtacttaaagacttatgctatatcagtaggccttggaccgtgtaccctacaaccaatactactaatgggtgagcttgtgaaagatgatgttgttgagaaagtgcactatccctcaaatatgtttgagcttgtatccttgagctggcgactaacaaacgacaccaaaacatatcaggctgaaaaggctcgaggacaacaagcctcaggcatagcatgctatatgaaggataatccaggagcaactgaggaagatgccattaagcacatatgtcgtgttgttgatcgggccttgaaagaagcaagctttgaatatttcaaaccatccaatgatatcccaatgggttgcaagtcctttatttttaaccttagattgtgtgtccaaatcttttacaagtttatagatgggtacggaatcgccaatgaggagattaaggactatataagaaaagtttatattgatccaattcaagtatga

SEQ ID NO: 33 - protein, 862 aa, Taxus brevifolia
MAQLSFNAALKMNALGNKAIHDPTNCRAKSERQMMWVCSRSGRTRVKMSRGSGGPGPVVMMSSSTGTSKVVSETSSTIVDDIPRLSANYHGDLWHHNVIQTLETPFRESSTYQERADELVVKIKDMFNALGDGDISPSAYDTAWVARLATISSDGSEKPRFPQALNWVFNNQLQDGSWGIESHFSLCDRLLNTTNSVIALSVWKTGHSQVQQGAEFIAENLRLLNEEDELSPDFQIIFPALLQKAKALGINLPYDLPFIKYLSTTREARLTDVSAAADNIPANMLNALEGLEEVIDWNKIMRFQSKDGSFLSSPASTACVLMNTGDEKCFTFLNNLLDKFGGCVPCMYSIDLLERLSLVDNIEHLGIGRHFKQEIKGALDYVYRHWSERGIGWGRDSLVPDLNTTALGLRTLRMHGYNVSSDVLNNFKDENGRFFSSAGQTHVELRSVVNLFRASDLAFPDERAMDDARKFAEPYLREALATKISTNTKLFKEIEYVVEYPWHMSIPRLEARSYIDSYDDNYVWQRKTLYRMPSLSNSKCLELAKLDFNIVQSLHQEELKLLTRWWKESGMADINFTRHRVAEVYFSSATFEPEYSATRIAFTKIGCLQVLFDDMADIFATLDELKSFTEGVKRWDTSLLHEIPECMQTCFKVWFKLMEEVNNDVVKVQGRDMLAHIRKPWELYFNCYVQEREWLEAGYIPTFEEYLKTYAISVGLGPCTLQPILLMGELVKDDVVEKVHYPSNMFELVSLSWRLTNDTKTYQAEKARGQQASGIACYMKDNPGATEEDAIKHICRVVDRALKEASFEYFKPSNDIPMGCKSFIFNLRLCVQIFYKFIDGYGIANEEIKDYIRKVYIDPIQV

SEQ ID NO: 34 - DNA, 2406 bp, Artificial Sequence
  Codon optimized sequence
tcttcatcaacaggcacttcaaaagtagtaagcgaaacatcttcaactattgtagacgatattccacgtctttcagcaaattatcatggtgatttatggcatcacaacgtaattcagactttagaaacaccatttagagaaagttcaacttatcaagagcgtgcagatgaattagtagtgaaaatcaaagatatgttcaatgcattaggtgacggtgacatctcaccttcagcttatgatactgcatgggtagctcgtgttgctaccatttcttctgatggtagcgaaaaaccacgttttcctcaagctcttaattgggtttttaacaatcaattacaagatggatcatggggtattgaatcacattttagtttatgcgatcgtttacttaatactacaaattcagttattgctttatcagtatggaaaactggtcactcacaggttcaacaaggtgccgaatttattgctgaaaatttacgtcttttaaatgaagaagacgaattaagtcctgattttcaaattatcttcccagctttattacagaaagccaaggctttaggaatcaatttaccctatgatttaccattcatcaaatatcttagtacaacacgcgaagctcgtttaacagatgtgtcagctgctgctgacaacataccagccaatatgcttaatgcacttgaaggtttagaagaagtgattgattggaataaaatcatgcgttttcaatctaaagatggttcatttttatcttctccagctagtacagcctgtgttttaatgaatacaggtgatgaaaaatgtttcacattcttaaataacttattagataaattcggcggttgtgttccatgtatgtatagcattgatttattagaacgtttatctttagtggacaacattgaacacttaggtattggtcgtcactttaaacaagaaatcaaaggtgcattagattatgtatatcgtcattggtctgaacgcggtatcggttggggtagagactctttagttccagatttaaacaccacagctttaggtttacgcacattaagaatgcacggttataacgtgtctagtgatgtacttaacaatttcaaagacgaaaatggtcgtttctttagtagtgctggtcaaacacacgtagagttacgttctgttgtaaatctttttcgcgcctcagatttagcctttccagacgaacgtgcaatggatgatgctcgtaaattcgcagaaccatatttacgtgaagcattagctacaaaaatatcaacaaatacaaagttattcaaagaaattgaatatgttgttgaatacccttggcacatgtcaattccacgtttagaagctcgtagttatattgacagttatgatgataattatgtatggcaacgtaagactttatatcgtatgccatcattaagtaattcaaaatgtttagaacttgctaaattagatttcaatattgttcaatctttacaccaagaagaacttaaacttttaactcgttggtggaaagaatctggtatggcagacataaatttcacccgccatcgtgtagctgaagtttacttttctagtgctacatttgagccagaatatagtgctactcgtattgcattcacaaaaattggttgcttacaagtacttttcgatgatatggctgacattttcgccactttagatgagttaaaaagttttactgaaggtgttaaacgctgggacacatcattattacatgaaattcccgaatgtatgcaaacttgttttaaagtatggtttaaacttatggaagaagtaaacaacgacgtagtaaaagttcaaggaagagatatgttagcacatattcgtaaaccctgggaattatactttaattgttatgttcaagaacgtgaatggttagaagctggttatattcctacattcgaagaatatcttaaaacttatgctattagtgtaggccttggtccttgtaccttacaacctattcttttaatgggtgagttagttaaagatgatgtagtagaaaaagttcattacccttctaacatgttcgaattagtttctttaagctggcgtttaactaatgataccaaaacatatcaagcagaaaaagtacgcggtcaacaagctagtggcattgcctgttatatgaaagacaatccaggtgctactgaagaagatgctattaaacacatttgtcgtgttgttgatcgtgcattaaaagaagcaagtttcgaatatttcaagccttcaaatgacattcctatgggttgtaaatcttttatctttaacttacgtttatgtgtacaaattttctataaattcattgatggttatggtatcgcaaacgaagaaattaaggactacattcgtaaggtttatattgatccaattcaagttggt

SEQ ID NO: 35 - DNA, 2496 bp, Artificial Sequence
  Codon optimized sequence
atggtaccatcttcatcaacaggcacttcaaaagtagtaagcgaaacatcttcaactattgtagacgatattccacgtctttcagcaaattatcatggtgatttatggcatcacaacgtaattcagactttagaaacaccatttagagaaagttcaacttatcaagagcgtgcagatgaattagtagtgaaaatcaaagatatgttcaatgcattaggtgacggtgacatctcaccttcagcttatgatactgcatgggtagctcgtgttgctaccatttcttctgatggtagcgaaaaaccacgttttcctcaagctcttaattgggtttttaacaatcaattacaagatggatcatggggtattgaatcacattttagtttatgcgatcgtttacttaatactacaaattcagttattgctttatcagtatggaaaactggtcactcacaggttcaacaaggtgccgaatttattgctgaaaatttacgtcttttaaatgaagaagacgaattaagtcctgattttcaaattatcttcccagctttattacagaaagccaaggctttaggaatcaatttaccctatgatttaccattcatcaaatatcttagtacaacacgcgaagctcgtttaacagatgtgtcagctgctgctgacaacataccagccaatatgcttaatgcacttgaaggtttagaagaagtgattgattggaataaaatcatgcgttttcaatctaaagatggttcatttttatcttctccagctagtacagcctgtgttttaatgaatacaggtgatgaaaaatgtttcacattcttaaataacttattagataaattcggcggttgtgttccatgtatgtatagcattgatttattagaacgtttatctttagtggacaacattgaacacttaggtattggtcgtcactttaaacaagaaatcaaaggtgcattagattatgtatatcgtcattggtctgaacgcggtatcggttggggtagagactctttagttccagatttaaacaccacagctttaggtttacgcacattaagaatgcacggttataacgtgtctagtgatgtacttaacaatttcaaagacgaaaatggtcgtttctttagtagtgctggtcaaacacacgtagagttacgttctgttgtaaatctttttcgcgcctcagatttagcctttccagacgaacgtgcaatggatgatgctcgtaaattcgcagaaccatatttacgtgaagcattagctacaaaaatatcaacaaatacaaagttattcaaagaaattgaatatgttgttgaatacccttggcacatgtcaattccacgtttagaagctcgtagttatattgacagttatgatgataattatgtatggcaacgtaagactttatatcgtatgccatcattaagtaattcaaaatgtttagaacttgctaaattagatttcaatattgttcaatctttacaccaagaagaacttaaacttttaactcgttggtggaaagaatctggtatggcagacataaatttcacccgccatcgtgtagctgaagtttacttttctagtgctacatttgagccagaatatagtgctactcgtattgcattcacaaaaattggttgcttacaagtacttttcgatgatatggctgacattttcgccactttagatgagttaaaaagttttactgaaggtgttaaacgctgggacacatcattattacatgaaattcccgaatgtatgcaaacttgttttaaagtatggtttaaacttatggaagaagtaaacaacgacgtagtaaaagttcaaggaagagatatgttagcacatattcgtaaaccctgggaattatactttaattgttatgttcaagaacgtgaatggttagaagctggttatattcctacattcgaagaatatcttaaaacttatgctattagtgtaggccttggtccttgtaccttacaacctattcttttaatgggtgagttagttaaagatgatgtagtagaaaaagttcattacccttctaacatgttcgaattagtttctttaagctggcgtttaactaatgataccaaaacatatcaagcagaaaaagtacgcggtcaacaagctagtggcattgcctgttatatgaaagacaatccaggtgctactgaagaagatgctattaaacacatttgtcgtgttgttgatcgtgcattaaaagaagcaagtttcgaatatttcaagccttcaaatgacattcctatgggttgtaaatcttttatctttaacttacgtttatgtgtacaaattttctataaattcattgatggttatggtatcgcaaacgaagaaattaaggactacattcgtaaggtttatattgatccaattcaagttggtaccggtgaaaacttatactttcaaggctcaggtggcggtggaagtgattacaaagatgatgatgataaaggaaccggttaa

SEQ ID NO: 36 - protein, 831 aa, Taxus brevifolia
  MISC_FEATURE (806)..(831): TEV-FLAG tag
MVPSSSTGTSKVVSETSSTIVDDIPRLSANYHGDLWHHNVIQTLETPFRESSTYQERADELVVKIKDMFNALGDGDISPSAYDTAWVARVATISSDGSEKPRFPQALNWVFNNQLQDGSWGIESHFSLCDRLLNTTNSVIALSVWKTGHSQVQQGAEFIAENLRLLNEEDELSPDFQIIFPALLQKAKALGINLPYDLPFIKYLSTTREARLTDVSAAADNIPANMLNALEGLEEVIDWNKIMRFQSKDGSFLSSPASTACVLMNTGDEKCFTFLNNLLDKFGGCVPCMYSIDLLERLSLVDNIEHLGIGRHFKQEIKGALDYVYRHWSERGIGWGRDSLVPDLNTTALGLRTLRMHGYNVSSDVLNNFKDENGRFFSSAGQTHVELRSVVNLFRASDLAFPDERAMDDARKFAEPYLREALATKISTNTKLFKEIEYVVEYPWHMSIPRLEARSYIDSYDDNYVWQRKTLYRMPSLSNSKCLELAKLDFNIVQSLHQEELKLLTRWWKESGMADINFTRHRVAEVYFSSATFEPEYSATRIAFTKIGCLQVLFDDMADIFATLDELKSFTEGVKRWDTSLLHEIPECMQTCFKVWFKLMEEVNNDVVKVQGRDMLAHIRKPWELYFNCYVQEREWLEAGYIPTFEEYLKTYAISVGLGPCTLQPILLMGELVKDDVVEKVHYPSNMFELVSLSWRLTNDTKTYQAEKVRGQQASGIACYMKDNPGATEEDAIKHICRVVDRALKEASFEYFKPSNDIPMGCKSFIFNLRLCVQIFYKFIDGYGIANEEIKDYIRKVYIDPIQVGTGENLYFQGSGGGGSDYKDDDDKGTG

SEQ ID NO: 37 - DNA, 1158 bp, Phomopsis amygdali
acacaattggaatggatgcgtcaaggactgccatctttggagtcatgtcctgtactggcaagaagccctgagatcgactcagacgaatctgcagtttcacccaccgcagatgaatcggactctacagaggatagcttgggaagcggaagtaggcaggattcttcgctgagcactgggttgtctttgtcgcctgttcacagcaacgaaggcaaggatttgcagagagtcgacaccgaccatatattcttcgagaaagcggtcctcgaggcgccctatgactacattgcttccatgccatctaaaggagtccgagatcaatttatcgatgctctgaacgactggttgcgtgttcctgatgtcaaggtgggaaagataaaggatgctgtccgtgttttgcacaactcttcgctgctgctcgacgacttccaagacaactctcccctaagacgcggcaaaccgtcgacgcataacatctttgggtcagcacagactgtgaatacggcgacttactcaataataaaagcaatcggccagatcatggaattttctgcaggcgaatctgtccaagaggtaatgaacagtattatgattttgtttcaaggccaagccatggatctcttctggacatataatggacacgtacccagtgaagaagaatattatcggatgatcgatcaaaaaaccgggcagctgttctcaatcgccaccagtcttcttctaaatgcagcagacaatgagattcccaggacgaaaattcaaagttgtcttcaccggctgacgcgtctacttggacgctgtttccagatacgtgacgattatcagaaccttgtttctgccgactacacaaagcagaagggtttctgcgaggatcttgatgaagggaaatggtctctagcgctgatccacatgattcacaaacagcggagtcatatggcattactcaatgtgctatcaacggggagaaagcatggtggcatgactttggagcagaagcagttcgtgttggacatcatagaggaggagaaaagtctggactataccagatccgtcatgatggacttgcacgttcagctgcgcgctgaaataggacggattgagattctgcttgattctcccaaccctgccatgaggcttttgctggagcttctgcgagtctga

SEQ ID NO: 38 - protein, 385 aa, Phomopsis amygdali
TQLEWMRQGLPSLESCPVLARSPEIDSDESAVSPTADESDSTEDSLGSGSRQDSSLSTGLSLSPVHSNEGKDLQRVDTDHIFFEKAVLEAPYDYIASMPSKGVRDQFIDALNDWLRVPDVKVGKIKDAVRVLHNSSLLLDDFQDNSPLRRGKPSTHNIFGSAQTVNTATYSIIKAIGQIMEFSAGESVQEVMNSIMILFQGQAMDLFWTYNGHVPSEEEYYRMIDQKTGQLFSIATSLLLNAADNEIPRTKIQSCLHRLTRLLGRCFQIRDDYQNLVSADYTKQKGFCEDLDEGKWSLALIHMIHKQRSHMALLNVLSTGRKHGGMTLEQKQFVLDIIEEEKSLDYTRSVMMDLHVQLRAEIGRIEILLDSPNPAMRLLLELLRV

SEQ ID NO: 39 - DNA, 1158 bp, Artificial Sequence
  Codon optimized sequence
atgacacaattagaatggatgcgtcaaggtttaccatcattagaatcatgtccagttttagctcgttcaccagaaattgattcagatgaatcagcagtttcaccaactgctgatgaatcagattcaacagaagattcattaggttcaggttcacgtcaagattcatcattatcaacaggtttatcattatcaccagttcattcaaatgaaggtaaagatttacaacgtgttgatacagatcatattttttttgaaaaagctgttttagaagctccatacgattatattgcttcaatgccatcaaaaggtgttcgtgaccaatttattgatgctttaaatgattggttacgtgttccagatgttaaagttggtaaaattaaagatgctgttcgtgttttacataattcatcattattattagatgattttcaagataattcaccattacgtcgtggtaaaccatcaacacataatatttttggttcagctcaaacagttaatacagctacatattcaattattaaagctattggtcaaattatggaattttctgctggtgagtcagttcaagaagttatgaactcaattatgattttatttcaaggtcaagctatggatttattttggacatataatggtcatgttccatcagaagaagaatattatcgtatgattgaccaaaaaacaggtcaattattttcaattgctacatcattattattaaatgctgctgataatgaaattccacgtacaaaaattcaatcatgtttacatcgtttaacacgtttattaggtcgttgttttcaaattcgtgatgattatcaaaatttagtttctgctgattacactaaacaaaaaggattctgtgaagatttagatgaaggtaaatggtcattagctttaattcacatgattcataaacaacgttcacacatggctttattaaatgttttatcaacaggtcgtaaacatggtggtatgacattagaacaaaaacaatttgttttagatattattgaagaagaaaaatcattagattatacacgttcagttatgatggatcttcatgttcaattacgtgctgaaattggtcgtattgaaattttattagattcaccaaatccagctatgcgtttattattagaattattacgtgtt

SEQ ID NO: 40 - DNA, 1197 bp, Artificial Sequence
  Codon optimized sequence
atgacacaattagaatggatgcgtcaaggtttaccatcattagaatcatgtccagttttagctcgttcaccagaaattgattcagatgaatcagcagtttcaccaactgctgatgaatcagattcaacagaagattcattaggttcaggttcacgtcaagattcatcattatcaacaggtttatcattatcaccagttcattcaaatgaaggtaaagatttacaacgtgttgatacagatcatattttttttgaaaaagctgttttagaagctccatacgattatattgcttcaatgccatcaaaaggtgttcgtgaccaatttattgatgctttaaatgattggttacgtgttccagatgttaaagttggtaaaattaaagatgctgttcgtgttttacataattcatcattattattagatgattttcaagataattcaccattacgtcgtggtaaaccatcaacacataatatttttggttcagctcaaacagttaatacagctacatattcaattattaaagctattggtcaaattatggaattttctgctggtgagtcagttcaagaagttatgaactcaattatgattttatttcaaggtcaagctatggatttattttggacatataatggtcatgttccatcagaagaagaatattatcgtatgattgaccaaaaaacaggtcaattattttcaattgctacatcattattattaaatgctgctgataatgaaattccacgtacaaaaattcaatcatgtttacatcgtttaacacgtttattaggtcgttgttttcaaattcgtgatgattatcaaaatttagtttctgctgattacactaaacaaaaaggattctgtgaagatttagatgaaggtaaatggtcattagctttaattcacatgattcataaacaacgttcacacatggctttattaaatgttttatcaacaggtcgtaaacatggtggtatgacattagaacaaaaacaatttgttttagatattattgaagaagaaaaatcattagattatacacgttcagttatgatggatcttcatgttcaattacgtgctgaaattggtcgtattgaaattttattagattcaccaaatccagctatgcgtttattattagaattattacgtgttaccggtagtgcttggtcacaccctcaatttgagaaataa

SEQ ID NO: 41 - protein, 398 aa, Phomopsis amygdali
  MISC_FEATURE (387)..(398): Strep tag II
MTQLEWMRQGLPSLESCPVLARSPEIDSDESAVSPTADESDSTEDSLGSGSRQDSSLSTGLSLSPVHSNEGKDLQRVDTDHIFFEKAVLEAPYDYIASMPSKGVRDQFIDALNDWLRVPDVKVGKIKDAVRVLHNSSLLLDDFQDNSPLRRGKPSTHNIFGSAQTVNTATYSIIKAIGQIMEFSAGESVQEVMNSIMILFQGQAMDLFWTYNGHVPSEEEYYRMIDQKTGQLFSIATSLLLNAADNEIPRTKIQSCLHRLTRLLGRCFQIRDDYQNLVSADYTKQKGFCEDLDEGKWSLALIHMIHKQRSHMALLNVLSTGRKHGGMTLEQKQFVLDIIEEEKSLDYTRSVMMDLHVQLRAEIGRIEILLDSPNPAMRLLLELLRVTGSAWSHPQFEK

SEQ ID NO: 42 - DNA, 32 bp, Artificial Sequence
  Primer
ggatccaataatggaatttaaatattcagaag

SEQ ID NO: 43 - DNA, 27 bp, Artificial Sequence
  Primer
gaattcttatttctcaaattgagggtg

SEQ ID NO: 44 - DNA, 1932 bp, Coccidioides immitis
atggcccacaagtattcgacgatcatcgactcttccacctacgacacgcaaggtctttgtcctggaatagatctcaggaggcacgtggctggcgacctcgaagaagtcggtgcgtttagggctcaggaagactggcgccgcttggttgggcccctagagaagccttatgcaggcctcttgggcccagactttagcttcatcactgccgcggtgcccgaatgtctcccagacagaatggagattaccgcttatgcgcttgaatttggtttcatgcatgacgacgtcatcgataaagagatccacaacgcatctttggacgaaatggagcatgctttggaacagggcggtcagaccggcaagatcgacgagaaagccgcttctggaaagcgtaaaatcgtcgctcagattctccgcgagatgatggcaattgaccctgagagagcaatgactgtcgccaagagctgggctgctggcgtccaacattccagtagacggcaggacgaaacgcactttaatactcttgaggagtacatcccttatagggccctcgacgtgggatacatgcgctggcatggtcttgtcacgtttggctgcgctattaccatccccgaggaagaggcggatgaggcgagggagcttctgaagcctgctttgatcactgcctctctcactaatgatctattctcattcgagaaggagcgcggtgacgccaatgttcaaaacgccatcttggttgtcatgagggagcacggctgtagcgaagaagaagcaagagagatttgtaaagagcgcatccgcgtcgaatgtgccaactatgtccgcgtggtcaagaacaccagggcacggacggatatcagtgatgaacttaagagatacatagaggtcatgcagtacacactttcaggaaacgctgcctggagtactaattgtccgagatacaacggaccaaccaaattcaatgagttgcagttgctgagagctgagtatggcctggagaaatacccggcaatgtggccaccgaaggatgcaactaacggccttcctgtcgaaaccgaacgtaaggagcctcttgtcaacggtaatgggcattatgcatcaaccaaggccaacggcctcaagaggaagaggaacggtagcggtacgggtgacgacacaaagaagaatggcactaaatgtgtcaagaagtcggcacagatatcgcaactgagcacggattcatttgctcttgcggatgtggtgtctttggccgttgatctgaatttaccagagctgagcgatgatgttgttctccaaccatatcgatacctcacctcccttccttctaagggtttccgtgaccaggccatagactccctcaacacatggcttaaagtgccccagaagtcggctaaaatgatcaagagcatcgtcaagatgctgcatagcgcatctctcatgcttgatgacatcgaagacgactcaccacttcgtcgtggtaggccctctactcacaacatctatggcaccgcccagacaatcaacagcgcgacgtaccaatatgtcaaagcgacaggtatggctaccgagctcggcaacccgtcatgccttcgcatcttcatcgaagagatgcaacagctgcatgtggggcagagctatgacctctactggacgcacaatacactatgcccgtccgtatcagagtatctgaaaatggttgatatgaagacgggtggcctattccgcatgctgacacgattgatggtcgccgaaagcccggtcggcgagaaggtgtcagacgacgctctgaacctgttgagttgcctcgtggggcgcttcttccagatccgcgacgactaccagaacctcgcttccgccgactacgctaagcagaagggctttgccgaggacctcgatgaagggaagctctccttcacgctgatccactgcatctga

SEQ ID NO: 45 - protein, 643 aa, Coccidioides immitis
MAHKYSTIIDSSTYDTQGLCPGIDLRRHVAGDLEEVGAFRAQEDWRRLVGPLEKPYAGLLGPDFSFITAAVPECLPDRMEITAYALEFGFMHDDVIDKEIHNASLDEMEHALEQGGQTGKIDEKAASGKRKIVAQILREMMAIDPERAMTVAKSWAAGVQHSSRRQDETHFNTLEEYIPYRALDVGYMRWHGLVTFGCAITIPEEEADEARELLKPALITASLTNDLFSFEKERGDANVQNAILVVMREHGCSEEEAREICKERIRVECANYVRVVKNTRARTDISDELKRYIEVMQYTLSGNAAWSTNCPRYNGPTKFNELQLLRAEYGLEKYPAMWPPKDATNGLPVETERKEPLVNGNGHYASTKANGLKRKRNGSGTGDDTKKNGTKCVKKSAQISQLSTDSFALADVVSLAVDLNLPELSDDVVLQPYRYLTSLPSKGFRDQAIDSLNTWLKVPQKSAKMIKSIVKMLHSASLMLDDIEDDSPLRRGRPSTHNIYGTAQTINSATYQYVKATGMATELGNPSCLRIFIEEMQQLHVGQSYDLYWTHNTLCPSVSEYLKMVDMKTGGLFRMLTRLMVAESPVGEKVSDDALNLLSCLVGRFFQIRDDYQNLASADYAKQKGFAEDLDEGKLSFTLIHCI

SEQ ID NO: 46 - DNA, 1929 bp, Artificial Sequence
  Codon optimized sequence
atggcacataaatacagtacaataattgactcttctacatacgatacacaaggcctttgtccaggtattgatttacgtagacacgtagctggtgatttagaagaagttggtgcatttcgtgctcaagaagattggcgtcgtttagtaggtccattagaaaaaccttatgcaggtttattaggtccagatttttctttcataactgctgctgttcctgaatgtttaccagaccgtatggaaattactgcttacgctttagaatttggttttatgcatgatgatgttatagataaagaaatacacaatgcttcattagatgaaatggagcatgctttagaacaaggtggtcaaacaggcaaaatcgacgagaaagcagcaagtggtaaacgtaaaattgtagctcaaattttacgtgaaatgatggctatagaccctgaacgtgctatgacagtagctaaaagttgggcagcaggtgttcaacatagtagtagacgtcaagatgaaacacatttcaacactttagaagaatacatcccataccgtgcattagatgttggttacatgcgttggcacggtttagttacattcggttgtgctatcactattcctgaggaagaagctgatgaagcacgtgaacttttaaaacctgctttaattactgctagtttaacaaacgatttattctcttttgaaaaagagcgtggtgatgcaaatgtacaaaatgcaattttagtagtaatgcgtgaacatggttgttctgaagaagaagctcgtgaaatctgtaaagaacgtattcgtgttgaatgcgctaattatgttcgtgtagttaaaaatacacgtgctcgtactgatatttctgacgagttaaaacgttatatagaagtaatgcaatacacattaagtggtaacgctgcttggtctactaattgtcctcgttataacggtcctacaaaattcaacgaattacaacttttacgtgctgaatatggtttagaaaaatatccagcaatgtggccaccaaaagacgctacaaacggtttacctgttgaaacagaacgtaaagaacctttagttaatggtaatggtcactacgcaagtactaaagctaatggcttaaaacgtaaaagaaatggttctggaacaggtgacgatactaaaaaaaacggtactaaatgtgtaaaaaaaagtgcacaaatttcacaactttctacagatagtttcgcattagcagatgttgtttctttagcagttgatcttaatcttccagaattaagtgacgatgttgttttacaaccatatcgttatttaacttcattaccttcaaaaggttttcgtgatcaggctattgacagtcttaatacatggttaaaagttccacaaaaatctgctaaaatgattaaatctatcgttaaaatgttacacagtgcaagtttaatgttagatgatattgaagacgatagtccattacgtcgtggtagaccatcaactcacaacatttacggtacagctcaaactattaactctgctacttatcagtacgtaaaagcaactggtatggcaacagaattaggtaatccttcttgtttacgtattttcatcgaagaaatgcaacaattacacgttggacaaagttatgacttatactggactcataatacattatgtccatctgtttctgagtacttaaaaatggtagacatgaaaactggtggtttatttcgtatgttaacacgtttaatggttgctgagtcaccagtaggagaaaaagttagtgatgatgcacttaatttacttagttgtttagttggacgtttcttccagattcgtgatgattaccaaaacttagcaagtgctgattacgctaaacaaaaaggttttgctgaagatttagatgaaggtaaattaagtttcactttaattcattgtatt

SEQ ID NO: 47 - DNA, 1968 bp, Artificial Sequence
  Codon optimized sequence
atggcacataaatacagtacaataattgactcttctacatacgatacacaaggcctttgtccaggtattgatttacgtagacacgtagctggtgatttagaagaagttggtgcatttcgtgctcaagaagattggcgtcgtttagtaggtccattagaaaaaccttatgcaggtttattaggtccagatttttctttcataactgctgctgttcctgaatgtttaccagaccgtatggaaattactgcttacgctttagaatttggttttatgcatgatgatgttatagataaagaaatacacaatgcttcattagatgaaatggagcatgctttagaacaaggtggtcaaacaggcaaaatcgacgagaaagcagcaagtggtaaacgtaaaattgtagctcaaattttacgtgaaatgatggctatagaccctgaacgtgctatgacagtagctaaaagttgggcagcaggtgttcaacatagtagtagacgtcaagatgaaacacatttcaacactttagaagaatacatcccataccgtgcattagatgttggttacatgcgttggcacggtttagttacattcggttgtgctatcactattcctgaggaagaagctgatgaagcacgtgaacttttaaaacctgctttaattactgctagtttaacaaacgatttattctcttttgaaaaagagcgtggtgatgcaaatgtacaaaatgcaattttagtagtaatgcgtgaacatggttgttctgaagaagaagctcgtgaaatctgtaaagaacgtattcgtgttgaatgcgctaattatgttcgtgtagttaaaaatacacgtgctcgtactgatatttctgacgagttaaaacgttatatagaagtaatgcaatacacattaagtggtaacgctgcttggtctactaattgtcctcgttataacggtcctacaaaattcaacgaattacaacttttacgtgctgaatatggtttagaaaaatatccagcaatgtggccaccaaaagacgctacaaacggtttacctgttgaaacagaacgtaaagaacctttagttaatggtaatggtcactacgcaagtactaaagctaatggcttaaaacgtaaaagaaatggttctggaacaggtgacgatactaaaaaaaacggtactaaatgtgtaaaaaaaagtgcacaaatttcacaactttctacagatagtttcgcattagcagatgttgtttctttagcagttgatcttaatcttccagaattaagtgacgatgttgttttacaaccatatcgttatttaacttcattaccttcaaaaggttttcgtgatcaggctattgacagtcttaatacatggttaaaagttccacaaaaatctgctaaaatgattaaatctatcgttaaaatgttacacagtgcaagtttaatgttagatgatattgaagacgatagtccattacgtcgtggtagaccatcaactcacaacatttacggtacagctcaaactattaactctgctacttatcagtacgtaaaagcaactggtatggcaacagaattaggtaatccttcttgtttacgtattttcatcgaagaaatgcaacaattacacgttggacaaagttatgacttatactggactcataatacattatgtccatctgtttctgagtacttaaaaatggtagacatgaaaactggtggtttatttcgtatgttaacacgtttaatggttgctgagtcaccagtaggagaaaaagttagtgatgatgcacttaatttacttagttgtttagttggacgtttcttccagattcgtgatgattaccaaaacttagcaagtgctgattacgctaaacaaaaaggttttgctgaagatttagatgaaggtaaattaagtttcactttaattcattgtattaccggttcagcttggtcacatccacaatttgagaaataa

SEQ ID NO: 48 - protein, 655 aa, Coccidioides immitis
  MISC_FEATURE (644)..(655): Strep tag II
MAHKYSTIIDSSTYDTQGLCPGIDLRRHVAGDLEEVGAFRAQEDWRRLVGPLEKPYAGLLGPDFSFITAAVPECLPDRMEITAYALEFGFMHDDVIDKEIHNASLDEMEHALEQGGQTGKIDEKAASGKRKIVAQILREMMAIDPERAMTVAKSWAAGVQHSSRRQDETHFNTLEEYIPYRALDVGYMRWHGLVTFGCAITIPEEEADEARELLKPALITASLTNDLFSFEKERGDANVQNAILVVMREHGCSEEEAREICKERIRVECANYVRVVKNTRARTDISDELKRYIEVMQYTLSGNAAWSTNCPRYNGPTKFNELQLLRAEYGLEKYPAMWPPKDATNGLPVETERKEPLVNGNGHYASTKANGLKRKRNGSGTGDDTKKNGTKCVKKSAQISQLSTDSFALADVVSLAVDLNLPELSDDVVLQPYRYLTSLPSKGFRDQAIDSLNTWLKVPQKSAKMIKSIVKMLHSASLMLDDIEDDSPLRRGRPSTHNIYGTAQTINSATYQYVKATGMATELGNPSCLRIFIEEMQQLHVGQSYDLYWTHNTLCPSVSEYLKMVDMKTGGLFRMLTRLMVAESPVGEKVSDDALNLLSCLVGRFFQIRDDYQNLASADYAKQKGFAEDLDEGKLSFTLIHCITGSAWSHPQFEK

SEQ ID NO: 49 - DNA, 2274 bp, Gibberella zeae
atggacttcacataccgctattcgttcgagcctacggactatgacactgacggtctctgtgatggtgttccggtccgtatgcacaagggtgcagacttggacgaggttgccatcttcaaagctcagtatgactgggagaagcatgttggtcctaagctgcccttccggggtgcattggggccaagacacaacttcatctgtcttactctgccggagtgcttgcctgagagactagagattgtgtcttatgccaatgagtttgccttccttcacgatgatattactgatgtcgagtcagctgagacgtcaaaggttgccgctgagaacgatgagttccttgatgcccttcaacaaggtgttagagaaggtgacatccagagccgtgagtccggaaagcgtcatctccaggcttggatcttcaagtccatggtggccattgaccgtgatagagctgtggccgctatgaacgcttgggccacctttatcaacacaggtgcaggatgcgcccacgatacaaacttcaagtcacttgatgagtatcttcactacagagctacagatgtcggttacatgttctggcacgctcttatcatcttcggatgcgccatcaccattcctgaacatgagattgagctatgccatcaactcgctcttccagccatcatgtccgtgactttgacaaacgacatctggtcatatggcaaagaagcagaggcagctgagaaatccggcaagcccggagattttgtcaacgctctcgttgttctgatgagagagcacaactgctccattgaagaagctgagcgtctctgcagagcgcgaaacaagatcgaagtagccaagtgtctccaagtcacaaaagagacacgagagcgaaaagatgtttcacaagatctcaaagactacctctaccatatgctgtttggtgtcagtggaaatgcgatctggagcactcagtgccgaagatatgacatgacagcgccttacaacgaaagacagcaggccagactcaagcagaccaaggatgagcttacttccacatatgatcctgttcaggctgccaaggaggccatgatggagtctactcgtcctgagatccacagactgcctactcccgatagtcccaggaaggagagctttgctgttcgtcctttggtgaatggcagtggacaatacaatggcaacaatcacatcaatggagtctccaatgaagttgacgtgcgtccttctattgagagacatgcctcaaccaagcgagctacttcagctgatgacatcgactggacggcacataagaaggttgatagtggggctgaccacaagaagaccctgtccgatatcatgctgcaagagttgcctcctatggaagacgatgtcgtcatggaaccataccgatatctgtgttctcttccctcaaagggagttagaaacaagaccattgacgctcttaacttctggctcaaggttcctattgagaatgcaaacaccatcaaggccatcactgaaagccttcatggatcatcacttatgcttgatgatatcgaggaccattcacaactgcgacgtggcaagccttcggcccacgctgtttttggtgaggcacagaccatcaactctgcaacatttcagtacattcagtctgttagcctgattagccagcttagaagccctaaggctttgaacatctttgttgatgagattcgacaacttttcatcggtcaggcttacgagctccagtggacctctaacatgatttgcccacctttggaggagtatttgcgaatggttgacggaaaaactggcgggttattccgtcttctcactcgtctcatggctgctgagtccactactgaggtagatgttgactttagccgtctgtgccagctttttggtcgctacttccagatccgagacgattacgccaacctcaagctcgcagactacaccgaacaaaagggtttctgtgaagaccttgacgagggcaagttctcactccctctcatcattgccttcaacgagaacaacaaggcccccaaagccgtagctcaactgcgcggcctcatgatgcagcgctgtgtcaacggcggcctcacctttgaacagaaggtgctagcactgaatctcattgaggaggctggtggaatttcgggcacggagaaggtgctgcactcactttatggtgagatggaggctgagctggaaaggttggctggtgtctttggggcggagaatcatcagcttgagcttattctggagatgctgcgtatagattag

SEQ ID NO: 50 - protein, 757 aa, Gibberella zeae
MDFTYRYSFEPTDYDTDGLCDGVPVRMHKGADLDEVAIFKAQYDWEKHVGPKLPFRGALGPRHNFICLTLPECLPERLEIVSYANEFAFLHDDITDVESAETSKVAAENDEFLDALQQGVREGDIQSRESGKRHLQAWIFKSMVAIDRDRAVAAMNAWATFINTGAGCAHDTNFKSLDEYLHYRATDVGYMFWHALIIFGCAITIPEHEIELCHQLALPAIMSVTLTNDIWSYGKEAEAAEKSGKPGDFVNALVVLMREHNCSIEEAERLCRARNKIEVAKCLQVTKETRERKDVSQDLKDYLYHMLFGVSGNAIWSTQCRRYDMTAPYNERQQARLKQTKDELTSTYDPVQAAKEAMMESTRPEIHRLPTPDSPRKESFAVRPLVNGSGQYNGNNHINGVSNEVDVRPSIERHASTKRATSADDIDWTAHKKVDSGADHKKTLSDIMLQELPPMEDDVVMEPYRYLCSLPSKGVRNKTIDALNFWLKVPIENANTIKAITESLHGSSLMLDDIEDHSQLRRGKPSAHAVFGEAQTINSATFQYIQSVSLISQLRSPKALNIFVDEIRQLFIGQAYELQWTSNMICPPLEEYLRMVDGKTGGLFRLLTRLMAAESTTEVDVDFSRLCQLFGRYFQIRDDYANLKLADYTEQKGFCEDLDEGKFSLPLIIAFNENNKAPKAVAQLRGLMMQRCVNGGLTFEQKVLALNLIEEAGGISGTEKVLHSLYGEMEAELERLAGVFGAENHQLELILEMLRID

SEQ ID NO: 51 - DNA, 2271 bp, Artificial Sequence
  Codon optimized sequence
atggactttacatatcgttatagttttgaaccaacagattatgatactgacggtctttgtgacggtgtaccagtaagaatgcacaaaggtgctgatttagacgaagttgctattttcaaagcacaatatgattgggaaaaacatgtaggccctaaattacctttccgtggtgcattaggtccacgtcataatttcatttgtttaactttaccagaatgtcttccagaaagattagaaatcgtttcttatgctaatgagttcgcatttttacatgatgatattactgatgtagaaagtgcagagacatcaaaagtagctgctgaaaacgatgaatttttagacgctttacaacaaggcgtacgtgagggagacattcaatctcgtgaatctggcaaacgtcacttacaagcatggattttcaaatctatggttgctattgacagagatcgtgctgttgcagctatgaatgcttgggcaactttcattaacactggtgctggttgtgcacacgacacaaatttcaaaagtttagatgaatatttacattatcgtgctactgacgtaggttatatgttctggcacgctttaatcatatttggttgtgcaatcacaatcccagaacatgaaattgaattatgccatcaattagcattaccagctattatgagtgttacattaacaaatgatatttggtcttatggtaaagaagcagaagctgcagaaaaatctggtaaaccaggtgattttgttaatgcacttgttgttttaatgcgtgaacacaattgttctatcgaagaagcagaacgtttatgtcgtgcaagaaacaaaattgaagttgcaaaatgtttacaagttactaaagaaacacgtgaacgtaaagatgtatcacaagatttaaaagactacttataccacatgttatttggagtatcaggtaacgctatttggtcaactcaatgccgtcgttacgatatgacagctccatataatgaacgtcaacaggcacgtttaaaacaaacaaaagatgaattaacatcaacttatgacccagttcaagcagctaaagaagcaatgatggaatctactcgtcctgaaattcacagattaccaacacctgattctcctcgtaaagagtcatttgctgttcgtccacttgttaacggatcaggtcaatataatggtaataatcacattaacggtgtttctaatgaagtagacgtacgtccatcaattgaacgtcatgctagtactaaacgtgctacatctgctgatgacattgattggacagctcataaaaaagtagatagtggtgctgatcacaaaaaaacattatcagacataatgcttcaagaacttccacctatggaggatgacgttgttatggaaccatatcgttacttatgttctcttccttcaaaaggagttcgtaataaaactatagatgcattaaacttttggttaaaagtacctattgaaaatgctaatactattaaagcaattacagaaagtttacacggttcttcacttatgttagatgatattgaagatcactctcaattaagacgtggtaaaccaagtgcacacgctgtatttggtgaagctcaaacaattaacagtgctacattccaatatatacagagtgtttctttaatttctcaattacgtagtccaaaagcattaaacatttttgtagatgaaattcgtcaactttttattggccaagcatacgaattacaatggacttctaatatgatttgtcctccattagaagaatacttaagaatggttgacggaaaaacaggtggtttatttcgtcttttaactcgtttaatggctgcagaaagtacaacagaagttgatgtagatttcagtcgtttatgtcaactttttggacgttactttcaaattcgtgatgattatgcaaacttaaaacttgcagattacactgaacagaaaggtttttgtgaagatttagatgaaggaaaattcagtttacctcttattatcgcttttaatgaaaacaataaagctccaaaagcagttgctcaattacgtggtttaatgatgcaacgttgtgtaaatggtggtttaacatttgaacaaaaagtattagctcttaaccttattgaagaagctggtggcatttctggtacagaaaaagtattacatagtttatacggtgaaatggaggctgaattagagagattagcaggagtatttggtgcagaaaaccaccaattagagttaattcttgaaatgttacgtattgat

SEQ ID NO: 52 - DNA, 2328 bp, Artificial Sequence
  Codon optimized sequence
atggactttacatatcgttatagttttgaaccaacagattatgatactgacggtctttgtgacggtgtaccagtaagaatgcacaaaggtgctgatttagacgaagttgctattttcaaagcacaatatgattgggaaaaacatgtaggccctaaattacctttccgtggtgcattaggtccacgtcataatttcatttgtttaactttaccagaatgtcttccagaaagattagaaatcgtttcttatgctaatgagttcgcatttttacatgatgatattactgatgtagaaagtgcagagacatcaaaagtagctgctgaaaacgatgaatttttagacgctttacaacaaggcgtacgtgagggagacattcaatctcgtgaatctggcaaacgtcacttacaagcatggattttcaaatctatggttgctattgacagagatcgtgctgttgcagctatgaatgcttgggcaactttcattaacactggtgctggttgtgcacacgacacaaatttcaaaagtttagatgaatatttacattatcgtgctactgacgtaggttatatgttctggcacgctttaatcatatttggttgtgcaatcacaatcccagaacatgaaattgaattatgccatcaattagcattaccagctattatgagtgttacattaacaaatgatatttggtcttatggtaaagaagcagaagctgcagaaaaatctggtaaaccaggtgattttgttaatgcacttgttgttttaatgcgtgaacacaattgttctatcgaagaagcagaacgtttatgtcgtgcaagaaacaaaattgaagttgcaaaatgtttacaagttactaaagaaacacgtgaacgtaaagatgtatcacaagatttaaaagactacttataccacatgttatttggagtatcaggtaacgctatttggtcaactcaatgccgtcgttacgatatgacagctccatataatgaacgtcaacaggcacgtttaaaacaaacaaaagatgaattaacatcaacttatgacccagttcaagcagctaaagaagcaatgatggaatctactcgtcctgaaattcacagattaccaacacctgattctcctcgtaaagagtcatttgctgttcgtccacttgttaacggatcaggtcaatataatggtaataatcacattaacggtgtttctaatgaagtagacgtacgtccatcaattgaacgtcatgctagtactaaacgtgctacatctgctgatgacattgattggacagctcataaaaaagtagatagtggtgctgatcacaaaaaaacattatcagacataatgcttcaagaacttccacctatggaggatgacgttgttatggaaccatatcgttacttatgttctcttccttcaaaaggagttcgtaataaaactatagatgcattaaacttttggttaaaagtacctattgaaaatgctaatactattaaagcaattacagaaagtttacacggttcttcacttatgttagatgatattgaagatcactctcaattaagacgtggtaaaccaagtgcacacgctgtatttggtgaagctcaaacaattaacagtgctacattccaatatatacagagtgtttctttaatttctcaattacgtagtccaaaagcattaaacatttttgtagatgaaattcgtcaactttttattggccaagcatacgaattacaatggacttctaatatgatttgtcctccattagaagaatacttaagaatggttgacggaaaaacaggtggtttatttcgtcttttaactcgtttaatggctgcagaaagtacaacagaagttgatgtagatttcagtcgtttatgtcaactttttggacgttactttcaaattcgtgatgattatgcaaacttaaaacttgcagattacactgaacagaaaggtttttgtgaagatttagatgaaggaaaattcagtttacctcttattatcgcttttaatgaaaacaataaagctccaaaagcagttgctcaattacgtggtttaatgatgcaacgttgtgtaaatggtggtttaacatttgaacaaaaagtattagctcttaaccttattgaagaagctggtggcatttctggtacagaaaaagtattacatagtttatacggtgaaatggaggctgaattagagagattagcaggagtatttggtgcagaaaaccaccaattagagttaattcttgaaatgttacgtattgataccggttctgcatggagtcatcctcaatttgagaaataatctagactcgagccttgg

SEQ ID NO: 53 - protein, 769 aa, Gibberella Zeae
  MISC_FEATURE (758)..(769): Strep tag II
MDFTYRYSFEPTDYDTDGLCDGVPVRMHKGADLDEVAIFKAQYDWEKHVGPKLPFRGALGPRHNFICLTLPECLPERLEIVSYANEFAFLHDDITDVESAETSKVAAENDEFLDALQQGVREGDIQSRESGKRHLQAWIFKSMVAIDRDRAVAAMNAWATFINTGAGCAHDTNFKSLDEYLHYRATDVGYMFWHALIIFGCAITIPEHEIELCHQLALPAIMSVTLTNDIWSYGKEAEAAEKSGKPGDFVNALVVLMREHNCSIEEAERLCRARNKIEVAKCLQVTKETRERKDVSQDLKDYLYHMLFGVSGNAIWSTQCRRYDMTAPYNERQQARLKQTKDELTSTYDPVQAAKEAMMESTRPEIHRLPTPDSPRKESFAVRPLVNGSGQYNGNNHINGVSNEVDVRPSIERHASTKRATSADDIDWTAHKKVDSGADHKKTLSDIMLQELPPMEDDVVMEPYRYLCSLPSKGVRNKTIDALNFWLKVPIENANTIKAITESLHGSSLMLDDIEDHSQLRRGKPSAHAVFGEAQTINSATFQYIQSVSLISQLRSPKALNIFVDEIRQLFIGQAYELQWTSNMICPPLEEYLRMVDGKTGGLFRLLTRLMAAESTTEVDVDFSRLCQLFGRYFQIRDDYANLKLADYTEQKGFCEDLDEGKFSLPLIIAFNENNKAPKAVAQLRGLMMQRCVNGGLTFEQKVLALNLIEEAGGISGTEKVLHSLYGEMEAELERLAGVFGAENHQLELILEMLRIDTGSAWSHPQFEK

SEQ ID NO: 54 - DNA, 2157 bp, Aspergillus clavatus
atggcctgcaagtactcgacactcatcgactcctccctgtacgacagggaaggtctttgccccggaattgatctcaggagacatgtcgccggtgagcttgaagaggtcggtgctttcagggcccaagaagactggcgccgtttggttggtccccttccaaagccttatgcgggcctcttaggacccgactttagcttcataaccggcgcggtgccagagtgtcacccagatagaatggagatcgtcgcttatgcgctggagtttggtttcatgcatgacgatgtcatcgatacggatgtcaaccatgcctcattggatgaggtgggacataccttggatcaaagtcgaactggcaaaatcgaagacaagggctccgatggaaagcgccaaatggtcactcaaatcatccgcgaaatgatggcaattgatccagagagagcgatgactgtagcgaagagctgggcctccggcgtccgacattcaagcagacggaaggaggacacgaactttaaggcacttgagcagtatataccctacagggccctcgacgtcgggtacatgctctggcacggcctggtcacctttggctgcgcaattacaattcccaacgaagaagaagaagaggcaaagaggctcatcatacctgcgttagtccaagcgtcgctgctgaacgaccttttctccttcgagaaggaaaagaacgacgctaatgtccagaacgctgtcttgattgtcatgaatgagcatgggtgtagcgaagaagaagcaagagatatcctcaagaaacgcatccgccttgaatgtgccaactacctccgcaatgtcaaagagaccaatgcgcgggcggatgtcagtgatgagttgaagaggtacatcaatgtcatgcagtataccctttccggcaacgcagcctggagtacgaattgcccgcggtacaacggaccaaccaagtttaatgagttgcagttgctgagaagcgagcacggcctggcaaaatacccgtcaaggtggtcacaggagaacagaaccagcggcctcgttgagggtgattgccacgaatccaagccaaacgagctcaagaggaagaggaatggcgtcagtgtagatgacgaaatgaggacgaatggcactaatggcgccaagaagccagcgcatgtctcgcaaccaagcacggattcgattgttctagaggatatggtgcagttggcgcgtacttgcgatttaccggacttgagtgatacagttattctccaaccataccggtaccttacctccctcccctctaagggtttccgagaccaagccatagactccatcaataaatggctgaaggtgcccccgaagtcggtgaagatgatcaaagacgtcgtcaagatgctgcatagtgcatctctcatgctcgatgatctcgaagacaactctccattacgtcgtggcaagccctctacccatagtatctacggcatggcccagacagtcaatagcgcaacgtaccaatacatcacagctacagatataaccgcccaactccagaactcagaaacctttcatatcttcgttgaagagttacagcagctgcacgtggggcagagctacgacctctactggacgcacaacacgctctgcccaaccatcgctgagtatttgaaaatggttgacatgaagacgggcggtctatttcgcatgttgacgcggatgatgatcgccgagagcccggtcgtcgataaggttcccaacagtgatatgaatttgtttagttgcctcattggacgcttcttccagatccgcgacgactatcaaaatctcgcttcagctgactacgcaaaggcgaaggggttcgccgaggatctcgacgaagggaaatattccttcacgctgatccactgcattcagactctggagtcaaagcccgagctcgcaggggagatgatgcagttgcgggcattccttatgaaaagaaggcatgaaggcaaacttagccaagaggctaagcaagaggtgttagtaaccatgaagaaaacagaaagcttgcaatacacgctcagcgttctgcgggaactgcacagcgagttggagaaggaagttgaaaatttagaggcgaagtttggcgaggagaacttcactcttagagtgatgctagagttgctgaaggtgtaa

SEQ ID NO: 55 - protein, 718 aa, Aspergillus clavatus
MACKYSTLIDSSLYDREGLCPGIDLRRHVAGELEEVGAFRAQEDWRRLVGPLPKPYAGLLGPDFSFITGAVPECHPDRMEIVAYALEFGFMHDDVIDTDVNHASLDEVGHTLDQSRTGKIEDKGSDGKRQMVTQIIREMMAIDPERAMTVAKSWASGVRHSSRRKEDTNFKALEQYIPYRALDVGYMLWHGLVTFGCAITIPNEEEEEAKRLIIPALVQASLLNDLFSFEKEKNDANVQNAVLIVMNEHGCSEEEARDILKKRIRLECANYLRNVKETNARADVSDELKRYINVMQYTLSGNAAWSTNCPRYNGPTKFNELQLLRSEHGLAKYPSRWSQENRTSGLVEGDCHESKPNELKRKRNGVSVDDEMRTNGTNGAKKPAHVSQPSTDSIVLEDMVQLARTCDLPDLSDTVILQPYRYLTSLPSKGFRDQAIDSINKWLKVPPKSVKMIKDVVKMLHSASLMLDDLEDNSPLRRGKPSTHSIYGMAQTVNSATYQYITATDITAQLQNSETFHIFVEELQQLHVGQSYDLYWTHNTLCPTIAEYLKMVDMKTGGLFRMLTRMMIAESPVVDKVPNSDMNLFSCLIGRFFQIRDDYQNLASADYAKAKGFAEDLDEGKYSFTLIHCIQTLESKPELAGEMMQLRAFLMKRRHEGKLSQEAKQEVLVTMKKTESLQYTLSVLRELHSELEKEVENLEAKFGEENFTLRVMLELLKV

SEQ ID NO: 56 - DNA, 2154 bp, Artificial Sequence
  Codon optimized sequence
atggcatgtaaatatagtactttaattgattcatctctttatgatcgtgaaggtttatgtcctggtattgacttacgtagacatgttgcaggtgaattagaagaagtaggtgctttccgtgcacaagaagactggcgtcgtcttgttggtcctttaccaaaaccatacgctggattattaggtcctgattttagttttattacaggagcagttccagaatgtcatccagatcgtatggaaattgttgcttatgctttagaatttggttttatgcacgatgatgttattgatacagacgtaaaccatgcttcattagacgaagttggtcacacattagatcaaagtcgtactggaaaaatagaagataaaggttcagatggtaaacgtcaaatggtaacacaaataattcgtgaaatgatggctattgatccagaaagagctatgacagtagcaaaaagttgggcttctggtgtacgtcacagtagtcgtcgtaaagaagatacaaacttcaaagcattagaacaatacattccatatagagctttagacgttggatatatgttatggcacggtcttgttacatttggctgtgcaatcactattcctaatgaggaagaagaagaagctaaacgtttaattatcccagctttagtacaagcaagtttacttaatgatttattctctttcgagaaagaaaaaaatgatgcaaacgtacagaacgcagtacttatagtaatgaatgagcacggttgttcagaggaagaagctcgtgatatacttaaaaaacgtatccgtttagaatgtgctaactacttacgtaatgttaaagaaacaaacgcacgtgcagatgtaagtgacgaattaaaacgttatatcaatgtaatgcaatatacattatcaggtaacgctgcttggtcaactaattgtccacgttataatggtccaacaaaattcaatgaattacaattattacgtagtgaacatggtttagcaaaatatccttctcgttggtcacaagaaaatcgtacaagtggtttagtagaaggcgactgtcatgaatcaaaacctaacgaacttaaacgtaaacgtaacggtgtatctgttgatgatgaaatgcgtacaaatggtacaaatggtgctaaaaaaccagctcatgtttctcaaccttcaacagactctattgttttagaagatatggttcaattagcacgtacttgtgatttacctgatcttagtgatacagttattttacaaccatatcgttatttaacaagtcttccatctaaaggttttcgtgatcaagcaattgattctattaacaaatggttaaaagtaccacctaaaagtgttaaaatgattaaagacgttgttaaaatgcttcactctgctagtttaatgttagatgacttagaagataacagtccattacgtcgtggtaaaccatcaacacactctatttacggtatggcacaaacagtaaattcagctacatatcaatacattacagctacagacatcacagcacaattacaaaattctgaaacattccatatttttgttgaagagcttcaacaattacatgttggtcagtcatacgatctttattggacacacaacactttatgtcctactattgcagagtatcttaaaatggtagatatgaaaacaggtggactttttcgtatgttaacaagaatgatgattgctgaatctccagtagttgataaagttccaaattcagacatgaacttattttcttgtttaattggtcgtttcttccaaatacgtgatgattatcaaaatttagcaagtgctgattatgctaaagcaaaaggttttgcagaagatttagatgaaggtaaatattcatttacacttatacactgtattcagacacttgaaagtaaacctgaacttgctggtgaaatgatgcagttacgtgcattcttaatgaaacgtcgtcatgagggtaaattatcacaagaggctaaacaagaagttttagtaactatgaaaaaaacagaatctttacaatacacattatctgttcttcgtgaattacattcagagttagaaaaagaagtagaaaatcttgaagctaaatttggtgaagaaaacttcactttacgtgttatgttagaattacttaaagtt

SEQ ID NO: 57 - DNA, 2211 bp, Artificial Sequence
  Codon optimized sequence
atggcatgtaaatatagtactttaattgattcatctctttatgatcgtgaaggtttatgtcctggtattgacttacgtagacatgttgcaggtgaattagaagaagtaggtgctttccgtgcacaagaagactggcgtcgtcttgttggtcctttaccaaaaccatacgctggattattaggtcctgattttagttttattacaggagcagttccagaatgtcatccagatcgtatggaaattgttgcttatgctttagaatttggttttatgcacgatgatgttattgatacagacgtaaaccatgcttcattagacgaagttggtcacacattagatcaaagtcgtactggaaaaatagaagataaaggttcagatggtaaacgtcaaatggtaacacaaataattcgtgaaatgatggctattgatccagaaagagctatgacagtagcaaaaagttgggcttctggtgtacgtcacagtagtcgtcgtaaagaagatacaaacttcaaagcattagaacaatacattccatatagagctttagacgttggatatatgttatggcacggtcttgttacatttggctgtgcaatcactattcctaatgaggaagaagaagaagctaaacgtttaattatcccagctttagtacaagcaagtttacttaatgatttattctctttcgagaaagaaaaaaatgatgcaaacgtacagaacgcagtacttatagtaatgaatgagcacggttgttcagaggaagaagctcgtgatatacttaaaaaacgtatccgtttagaatgtgctaactacttacgtaatgttaaagaaacaaacgcacgtgcagatgtaagtgacgaattaaaacgttatatcaatgtaatgcaatatacattatcaggtaacgctgcttggtcaactaattgtccacgttataatggtccaacaaaattcaatgaattacaattattacgtagtgaacatggtttagcaaaatatccttctcgttggtcacaagaaaatcgtacaagtggtttagtagaaggcgactgtcatgaatcaaaacctaacgaacttaaacgtaaacgtaacggtgtatctgttgatgatgaaatgcgtacaaatggtacaaatggtgctaaaaaaccagctcatgtttctcaaccttcaacagactctattgttttagaagatatggttcaattagcacgtacttgtgatttacctgatcttagtgatacagttattttacaaccatatcgttatttaacaagtcttccatctaaaggttttcgtgatcaagcaattgattctattaacaaatggttaaaagtaccacctaaaagtgttaaaatgattaaagacgttgttaaaatgcttcactctgctagtttaatgttagatgacttagaagataacagtccattacgtcgtggtaaaccatcaacacactctatttacggtatggcacaaacagtaaattcagctacatatcaatacattacagctacagacatcacagcacaattacaaaattctgaaacattccatatttttgttgaagagcttcaacaattacatgttggtcagtcatacgatctttattggacacacaacactttatgtcctactattgcagagtatcttaaaatggtagatatgaaaacaggtggactttttcgtatgttaacaagaatgatgattgctgaatctccagtagttgataaagttccaaattcagacatgaacttattttcttgtttaattggtcgtttcttccaaatacgtgatgattatcaaaatttagcaagtgctgattatgctaaagcaaaaggttttgcagaagatttagatgaaggtaaatattcatttacacttatacactgtattcagacacttgaaagtaaacctgaacttgctggtgaaatgatgcagttacgtgcattcttaatgaaacgtcgtcatgagggtaaattatcacaagaggctaaacaagaagttttagtaactatgaaaaaaacagaatctttacaatacacattatctgttcttcgtgaattacattcagagttagaaaaagaagtagaaaatcttgaagctaaatttggtgaagaaaacttcactttacgtgttatgttagaattacttaaagttaccggtagtgcttggagtcatcctcaattcgagaaataatctagactcgagccttgg

SEQ ID NO: 58 - protein, 730 aa, Aspergillus clavatus
  MISC_FEATURE (719)..(730): Strep tag II
MACKYSTLIDSSLYDREGLCPGIDLRRHVAGELEEVGAFRAQEDWRRLVGPLPKPYAGLLGPDFSFITGAVPECHPDRMEIVAYALEFGFMHDDVIDTDVNHASLDEVGHTLDQSRTGKIEDKGSDGKRQMVTQIIREMMAIDPERAMTVAKSWASGVRHSSRRKEDTNFKALEQYIPYRALDVGYMLWHGLVTFGCAITIPNEEEEEAKRLIIPALVQASLLNDLFSFEKEKNDANVQNAVLIVMNEHGCSEEEARDILKKRIRLECANYLRNVKETNARADVSDELKRYINVMQYTLSGNAAWSTNCPRYNGPTKFNELQLLRSEHGLAKYPSRWSQENRTSGLVEGDCHESKPNELKRKRNGVSVDDEMRTNGTNGAKKPAHVSQPSTDSIVLEDMVQLARTCDLPDLSDTVILQPYRYLTSLPSKGFRDQAIDSINKWLKVPPKSVKMIKDVVKMLHSASLMLDDLEDNSPLRRGKPSTHSIYGMAQTVNSATYQYITATDITAQLQNSETFHIFVEELQQLHVGQSYDLYWTHNTLCPTIAEYLKMVDMKTGGLFRMLTRMMIAESPVVDKVPNSDMNLFSCLIGRFFQIRDDYQNLASADYAKAKGFAEDLDEGKYSFTLIHCIQTLESKPELAGEMMQLRAFLMKRRHEGKLSQEAKQEVLVTMKKTESLQYTLSVLRELHSELEKEVENLEAKFGEENFTLRVMLELLKVTGSAWSHPQFEK

SEQ ID NO: 59 - DNA, 26 bp, Artificial Sequence
  Primer
caccatggaatttaaatattcagaag

SEQ ID NO: 60 - DNA, 21 bp, Artificial Sequence
  Primer
ttatttctcaaattgagggtg

(19) United States
(12) Patent Application Publication
(10) Pub. No.: US 2015/0010978 A1
(43) Pub. Date: Jan. 8, 2015

(54) TERPENE AND TERPENOID PRODUCTION IN PROKARYOTES AND EUKARYOTES

(71) Applicant: Sapphire Energy, Inc., San Diego, CA (US)

(72) Inventors: NICOLE A HEAPS, San Diego, CA (US); Craig A Behnke, San Diego, CA (US); David Molina, San Diego, CA (US)

(73) Assignee: Sapphire Energy, Inc., San Diego, CA (US)

(21) Appl. No.: 14/472,028

(22) Filed: Aug. 28, 2014

Related U.S. Application Data
(63) Continuation of application No. 13/255,888, filed on Nov. 9, 2011.
(60) Provisional application No. 61/159,366, filed on Mar. 11, 2009.

(51) Int. Cl. C12P 15/00; C12N 15/70; C12N 15/82; C12N 15/74

(52) U.S. Cl. CPC C12P 15/00; C12N 15/74; C12N 15/70; C12N 15/8249
USPC 435/167; 435/252.3; 435/254.2; 435/257.2; 435/166; 435/252.33; 435/254.21; 435/254.22; 435/252.31

(57) ABSTRACT

Terpene synthases are enzymes that directly convert IPP & DMAPP to terpenes, such as fusicoccadiene. Described herein are methods and compositions for the production of terpenes and terpenoids for use as fuel molecules or other useful components. Genetically engineered enzymes capable of producing terpenes and terpenoids are also described.

### CROSS REFERENCE TO RELATED APPLICATIONS

This application is a continuation of U.S. patent application Ser. No. 13/255,888 filed Nov. 9, 2011, which is the national phase of International Patent Application Number PCT/US2010/026445 filed Mar. 5, 2010, which claims the benefit of U.S. Provisional Application No. 61/159,366, filed Mar. 11, 2009, each of which is incorporated by reference in its entirety for all purposes.

### INCORPORATION BY REFERENCE

All publications, patents, patent applications, public databases, public database entries, and other references cited in this application are herein incorporated by reference in their entirety as if each individual publication, patent, patent application, public database, public database entry, or other reference was specifically and individually indicated to be incorporated by reference.

### BACKGROUND

Products, such as oil, petrochemicals, and other substances useful for the production of petrochemicals are increasingly in demand. Much of today's fuel products are generated from fossil fuels, which are not considered renewable energy sources, as they are the result of organic material being covered by successive layers of sediment over the course of millions of years. There is also a growing desire to lessen dependence on imported crude oil. Public awareness regarding pollution and environmental hazards has also increased. As a result, there has been a growing interest and need for alternative methods to produce fuel products. Thus, there exists a pressing need for alternative methods to develop fuel products that are renewable, sustainable, and less harmful to the environment.

Liquid fuels (gasoline, diesel, jet fuel, and kerosene, for example) are primarily composed of mixtures of paraffinic and aromatic hydrocarbons. Terpenes are a class of biologically produced molecules synthesized from five carbon precursor molecules in a wide range of organisms. Terpenes are pure hydrocarbons, while terpenoids may contain one or more oxygen atoms. Because terpenes are hydrocarbons with a low oxygen content and contain no nitrogen or other heteroatoms, terpenes can be used as fuel components with minimal processing.

Examples of terpenes are fusicoccadiene, casbene, ent-kaurene, taxadiene, and abietadiene.

Described herein are methods and compositions for the production of terpenes and terpenoids for use as fuel molecules or components.

### SUMMARY

1. An isolated polynucleotide capable of transforming a photosynthetic bacterium, a yeast, an alga, or a vascular plant, wherein the polynucleotide comprises a nucleic acid sequence of SEQ ID NO: 1, SEQ ID NO:4, SEQ ID NO: 7, SEQ ID NO: 9, SEQ ID NO: 11, SEQ ID NO: 15, SEQ ID NO: 17, SEQ ID NO: 21, SEQ ID NO: 26, SEQ ID NO: 28, SEQ ID NO: 32, SEQ ID NO: 34, SEQ ID NO: 37, SEQ ID NO: 39, SEQ ID NO: 44, SEQ ID NO: 46, SEQ ID NO: 49, SEQ ID NO: 51, SEQ ID NO: 54, or SEQ ID NO: 56. 2. The isolated polynucleotide of claim 1, wherein the polynucleotide comprises a nucleic acid sequence of SEQ ID NO: 4, SEQ ID NO: 7, SEQ ID NO: 11, SEQ ID NO: 17, SEQ ID NO: 21, SEQ ID) NO: 28, SEQ ID NO: 34, or SEQ ID NO: 39. 3. The isolated polynucleotide of claim 1 or claim 2, wherein the polynucleotide further comprises a nucleic acid which facilitates homologous recombination into a genome of the photosynthetic bacterium, yeast, alga, or vascular plant. 4. The isolated polynucleotide of claim 3, wherein the genome is a chloroplast genome of the alga or the vascular plant. 5. The isolated polynucleotide of claim 3, wherein the genome is a nuclear genome of the yeast, the alga, or the vascular plant. 6. The isolated polynucleotide of claim 1, wherein the photosynthetic bacterium is a member of genera Synechocystis, genera Synechococcus, or genera Athrospira. 7. The isolated polynucleotide of claim 1, wherein the photosynthetic bacterium is a cyanobacterium. 8. The isolated polynucleotide of claim 1, wherein the alga is a microalga. 9. The isolated polynucleotide of claim 1, wherein the alga is C. reinhardtii, D. salina, H. pluvalis, S. dimorphus, D. viridis, D. tertiolecta, N. oculata, or N. salina. 10. The isolated polynucleotide of claim 1, wherein the alga is a cyanophyta, a prochlorophyta, a rhodophyta, a chlorophyta, a heterokontophyta, a tribophyta, a glaucophyta, a chlorarachniophyte, a euglenophyta, a euglenoid, a haptophyta, a chrysophyta, a cryptophyta, a cryptomonad, a dinophyta, a dinoflagellata, a pyrmrnnesiophyta, a bacillariophyta, a xanthophyta, a eustigmatophyta, a raphidophyta, a phaeophyta, or a phytoplankton. 11. The isolated polynucleotide of claim 1, wherein the polynucleotide further comprises a nucleic acid encoding a tag for purification or detection. 12. The isolated polynucleotide of claim 11, wherein the tag is a His-6 tag, a FLAG epitope, a c-myc epitope, a Strep-TAGII, a biotin tag, a glutathione S-transferase (GST), a chitin binding protein (CBP), a maltose binding protein (MBP), or a metal affinity tag. 13. The isolated polynucleotide of claim 1, wherein the polynucleotide further comprises a nucleic acid encoding an amino acid sequence of SEQ ID NO: 3, SEQ ID NO: 12, SEQ ID NO: 19, SEQ ID NO: 23, or SEQ ID NO: 29. 14. The isolated polynucleotide of claim 1, wherein the polynucleotide further comprises a nucleic acid encoding a selectable marker. 15. The isolated polynucleotide of claim 14, wherein the selectable marker is kanamycin, chloramphenicol, ampicillin, or glufosinate. 16. A bacterial, yeast, alga, or vascular plant cell comprising the isolated polynucleotide of any one of claims 1 to 15.

17. An isolated polynucleotide capable of transforming a photosynthetic bacterium a yeast, an alga, or a vascular plant, comprising a nucleic acid encoding a terpene synthase comprising, (a) an amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55; or (b) a homolog of the amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55. 18. The isolated polynucleotide of claim 17, wherein the homolog has at least 50%, at least 60%¹ at least 70%, at least 75%, at least 80%, at least 85%, at least 90%, at least 95%, at least 98%, or at least 99% sequence identity to the amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55. 19. The isolated polynucleotide of claim 17, wherein the terpene synthase comprises the amino acid sequence of SEQ ID NO: 2. 20. The isolated polynucleotide of claim 17, wherein the photosynthetic bacterium is a member of genera Synechocystis, genera Synechococcus, or genera Athrospira. 21. The isolated polynucleotide of claim 17, wherein the photosynthetic bacterium is a cyanobacterium. 22. The isolated polynucleotide of claim 17, wherein the alga is a microalga. 23. The isolated polynucleotide of claim 17, wherein the alga is C. reinhardtii, D. salina, H. pluvalis, S. dimorphus, D. viridis, D. tertiolecta, N. oculata, or N. salina. 24. The isolated polynucleotide of claim 17, wherein the alga is a cyanophyta, a prochlorophyta, a rhodophyta, a chlorophyta, a heterokontophyta, a tribophyta, a glaucophyta, a chlorarachniophyte, a euglenophyta, a euglenoid, a haptophyta, a chrysophyta, a cryptophyta, a cryptomonad, a dinophyta, a dinoflagellata, a pyrmnesiophyta, a bacillariophyta, a xanthophyta, a eustigmatophyta, a raphidophyta, a phaeophyta, or a phytoplankton. 25. A bacterial, yeast, alga, or vascular plant cell comprising the isolated polynucleotide of any one of claims 17 to 24.

26. A vector comprising a polynucleotide comprising a nucleic acid encoding a terpene synthase, wherein the terpene synthase cyclyzes a terpene, and wherein the terpene synthase is capable of being expressed in a photosynthetic bacterium, a yeast, an alga, or a vascular plant. 27. The vector of claim 26, wherein the nucleic acid is codon biased for expression in the photosynthetic bacterium, yeast, alga, or vascular plant. 28. The vector of claim 27, wherein the codon bias is hot codon bias. 29. The vector of claim 27, wherein the codon bias is regular codon bias. 30. The vector of claim 26, wherein the terpene synthase is a diterpene synthase. 31. The vector of claim 30, wherein the diterpene synthase is a fusicoccadiene synthase, a kaurene synthase, a casbene synthase, a taxadiene synthase, an abietadiene synthase, or a homolog of any one of the above. 32. The vector of claim 31, wherein the diterpene synthase is a fuisicoccadiene synthase or a homolog of a fusicoccadiene synthase, 33. The vector of claim 26, wherein the nucleic acid comprises a nucleotide sequence of SEQ ID NO: 1, SEQ ID NO:4, SEQ ID NO: 7, SEQ ID NO: 9, SEQ ID NO: 11, SEQ ID NO: 15, SEQ ID NO: 17, SEQ ID NO: 21, SEQ ID NO: 26, SEQ ID NO: 28, SEQ ID NO: 32, SEQ ID NO: 34, SEQ ID NO: 37, SEQ ID NO: 39, SEQ ID NO: 44, SEQ ID NO: 46, SEQ ID NO: 49, SEQ ID NO: 51, SEQ ID NO: 54, or SEQ ID NO: 56. 34. The vector of claim 26, wherein the nucleic acid comprises a nucleotide sequence of SEQ ID NO: 4, SEQ ID NO: 7, SEQ ID NO: 11, SEQ ID NO: 17, SEQ ID NO: 21, SEQ ID NO: 28, SEQ ID NO: 34, or SEQ ID NO: 39. 35. The vector of claim 26, wherein the nucleic acid encoding a terpene synthase comprises, (a) an amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55; or (b) a homolog of the amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55. 36. The vector of claim 35, wherein the homolog has at least 50%, at least 60%, at least 70%, at least 75%, at least 80%, at least 85%, at least 90%, at least 95%, at least 98%, or at least 99% sequence identity to the amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55. 37. The vector of claim 26, wherein the terpene synthase comprises an amino acid sequence of SEQ ID NO: 2. 38. The vector of claim 26, wherein the nucleic acid comprises a nucleotide sequence of SEQ ID. NO: 4 or SEQ ID. NO: 7. 39. The vector of claim 38, wherein the nucleic acid comprises the nucleotide sequence of SEQ ID. NO: 7. 40. The vector of claim 26, wherein the terpene is a diterpene, 41. The vector of claim 40, wherein the diterpene is a cyclical diterpene. 42. The vector of claim 26, wherein the terpene is a fusicoccadiene, a casbene, an ent-kaurene, a taxadiene, or an abietadiene. 43. The vector of claim 42, wherein the terpene is a fusicoccadiene. 44. The vector of claim 43, wherein the fusicoccadiene is fusicocca-2,10(14)-diene. 45. The vector of claim 26, wherein the terpene synthase is a fusion terpene synthase. 46. The vector of 45, wherein the fusion terpene synthase comprises a portion of a casbene synthase and a portion of a geranylgeranyl-diphosphate (GGPP) synthase. 47. The vector of 46, wherein the fusion terpene synthase comprises the amino acid sequence of SEQ ID NO: 22. 48. The vector of any one of claims 26-47, wherein the polynucleotide further comprises a promoter for expression in the photosynthetic bacterium, yeast, alga, or vascular plant. 49. The vector of claim 48, wherein the promoter is a constitutive promoter. 50. The vector of claim 48, wherein the promoter is an inducible promoter. 51. The vector of claim 50, wherein the inducible promoter is a light inducible promoter, a nitrate inducible promoter, or a heat responsive promoter. 52. The vector of claim 48, wherein the promoter is T7, psbD, psdA, tufA, ItrA, atpA, or tubulin. 53. The vector of claim 48, wherein the promoter is a chloroplast promoter. 54. The vector of claim 48, wherein the promoter is psbA, psbD, atpA, or tufA. 55. The vector of any one of claims 48 to 54, wherein the promoter is operably linked to the polynucleotide. 56. The vector of claim 26, wherein said vector further comprises a 5′ regulatory region. 57. The vector of claim 56, wherein said 5′ regulatory region further comprises a promoter. 58. The vector of claim 57, wherein said promoter is a constitutive promoter. 59. The vector of claim 57, wherein said promoter is an inducible promoter. 60. The vector of claim 59, wherein said inducible promoter is a light inducible promoter, nitrate inducible promoter, or a heat responsive promoter. 61. The vector of any one of claims 56 to 60, further comprising a 3′ regulatory region. 62. The vector of any one of claims 57 to 60, wherein the promoter is operably linked to the polynucleotide. 63. The vector of any one of claims 26 to 62, wherein the polynucleotide further comprises a nucleic acid which facilitates homologous recombination into a genome of the photosynthetic bacterium, yeast, alga, or vascular plant. 64. The vector of claim 63, wherein the genome is a chloroplast genome of the alga or the vascular plant. 65. The vector of claim 63, wherein the genome is a nuclear genome of the yeast, the alga, or the vascular plant. 66. The vector of claim 26, wherein the photosynthetic bacterium is a member of genera Synechocystis, genera Synechococcus, or genera Athrospira. 67. The vector of claim 26, wherein the photosynthetic bacterium is a cyanobacterium. 68. The vector of claim 26, wherein the alga is a microalga. 69. The vector of claim 26, wherein the alga is C. reinhardtii, D. salina, H. pluvalis, S. dimorphus, D. viridis, D. tertiolecta, N. oculata, or N. salina. 70. The vector of claim 26, wherein the alga is a cyanophyta, a prochlorophyta, a rhodophyta, a chlorophyta, a heterokontophyta, a tribophyta, a glaucophyta, a chlorarachniophyte, a euglenophyta, a euglenoid, a haptophyta, a chrysophyta, a cryptophyta, a cryptomonad, a dinophyta, a dinoflagellata, a pyrmnesiophyta, a bacillariophyta, a xanthophyta, a eustigmatophyta, a raphidophyta, a phaeophyta, or a phytoplankton. 71. The vector of claim 26, wherein the polynucleotide further comprises a nucleic acid encoding a tag for purification or detection of the terpene synthase. 72. The vector of claim 71, wherein the tag is a H-is-6 tag, a FLAG epitope, a c-myc epitope, a Strep-TAG II, a biotin tag, a glutathione S-transferase (GST), a chitin binding protein (CBP), a maltose binding protein (MBP), or a metal affinity tag. 73. The vector of claim 26, wherein the polynucleotide further comprises a nucleic acid encoding an amino acid sequence of SEQ ID NO: 3, SEQ ID NO: 12, SEQ ID NO: 19. SEQ ID NO: 23, or SEQ ID NO: 29. 74. The vector of claim 26, wherein the polynucleotide further comprises a nucleic acid encoding a selectable marker. 75. The vector of claim 74, wherein the selectable marker is kanamycin, chloramphenicol, ampicillin, or glufosinate. 76. The vector of claim 26, wherein the photosynthetic bacterium, yeast, alga, or vascular plant does not normally produce the terpene.

77. A vector comprising, a polynucleotide comprising a nucleic acid sequence of SEQ ID NO: 46, SEQ ID NO: 51, or SEQ ID NO: 56. 78. The vector of claim 77, wherein the nucleic acid sequence is operably linked to a promoter in a host organism. 79. The vector of claim 78, wherein the promoter is a constitutive promoter. 80. The vector of claim 78, wherein the promoter is an inducible promoter. 81. The vector of claim 80, wherein the inducible promoter is a light inducible promoter, a nitrate inducible promoter, or a heat responsive promoter. 82. The vector of claim 78, wherein the promoter is T7, psbD, psdA, tufA, ItrA, atpA, or tubulin. 83. The vector of claim 78, wherein the promoter is a chloroplast promoter. 84. The vector of claim 78, wherein the promoter is psbA, psbD, atpA, or tufA, 85. The vector of claim 78, wherein the organism is a photosynthetic bacterium, a yeast, an alga, or a vascular plant. 86. The vector of claim 85, wherein the photosynthetic bacterium is a member of genera Synechocystis, genera Synechococcus, or genera Athrospira. 87. The vector of claim 85, wherein the photosynthetic bacterium is a cyanobacterium. 88. The vector of claim 85, wherein the alga is a microalga. 89. The vector of claim 85, wherein the alga is C. reinhardtii, D. salina, H. pluvalis, S. dimorphus, D. viridis, D. tertiolecta, N. oculata, or N. salina. 90. The vector of claim 85, wherein the alga is a cyanophyta, a prochlorophyta, a rhodophyta, a chlorophyta, a heterokontophyta, a tribophyta, a glaucophyta, a chlorarachniophyte, a euglenophyta, a euglenoid, a haptophyta, a chrysophyta, a cryptophyta, a cryptomonad, a dinophyta, a dinoflagellata, a pyrmnesiophyta, a bacillariophyta, a xanthophyta, a eustigmatophyta, a raphidophyta, a phaeophyta, or a phytoplankton.

91. A vector comprising a polynucleotide comprising a nucleic acid encoding an enzyme capable of modulating a terpenoid biosynthetic pathway in an organism wherein the organism is a photosynthetic bacterium, a yeast, an alga., or a vascular plant. 92. The vector of claim 91, wherein the nucleic acid is codon biased for expression in the photosynthetic bacterium, yeast, alga, or vascular plant. 93. The vector of claim 92, wherein the codon bias is hot codon bias, 94. The vector of claim 92, wherein the codon bias is regular codon bias. 95. The vector of claim 91, wherein the enzyme is a terpene synthase. 96. The vector of claim 95, wherein the terpene synthase is a diterpene synthase. 97. The vector of claim 96, wherein the diterpene synthase is a fusicoccadiene synthase, a kaurene synthase, a casbene synthase, a taxadiene synthase, an abietadiene synthase, or a homolog of any one of the above. 98. The vector of claim 97, wherein the diterpene synthase is a fusicoccadiene synthase or a homolog of a fusicoccadiene synthase. 99. The vector of claim 91, wherein the nucleic acid comprises a nucleotide sequence of SEQ ID NO: 1, SEQ ID NO:4, SEQ ID NO: 7, SEQ ID NO: 9, SEQ ID NO: 11, SEQ ID NO: 15, SEQ ID NO: 17, SEQ ID NO: 21, SEQ ID NO: 26, SEQ ID NO: 28, SEQ ID NO: 32, SEQ ID NO: 34, SEQ ID NO: 37, SEQ ID NO: 39, SEQ ID NO: 44, SEQ ID NO: 46, SEQ ID NO: 49, SEQ ID NO: 51, SEQ ID NO: 54, or SEQ ID NO: 56. 100. The vector of claim 91, wherein the nucleic acid comprises a nucleotide sequence of SEQ ID NO: 4, SEQ ID NO: 7, SEQ ID NO: 11, SEQ ID NO: 17, SEQ ID NO: 21, SEQ ID NO: 28, SEQ ID NO: 34, or SEQ ID NO: 39. 101. The vector of claim 95, wherein the terpene synthase comprises, (a) an amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55;or (b) a homolog of the amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55. 102. The vector of claim 101, wherein the homolog has at least 50%, at least 60%, at least 70%, at least 75%, at least 80%, at least 85%, at least 90%, at least 95%, at least 98%, or at least 99% sequence identity to the amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55. 103. The vector of claim 95, wherein the terpene synthase is a fusion terpene synthase. 104. The vector of 103, wherein the fusion terpene synthase comprises a portion of a casbene synthase and a portion of a geranylgeranyl-diphosphate (GGPP) synthase. 105. The vector of 104, wherein the fusion terpene synthase comprises the amino acid sequence of SEQ ID NO: 22. 106. The vector of any one of claims 91-105, wherein the polynucleotide further comprises a promoter for expression in the photosynthetic bacterium, yeast, alga, or vascular plant. 107. The vector of claim 106, wherein the promoter is a constitutive promoter. 108. The vector of claim 106, wherein the promoter is an inducible promoter. 109. The vector of claim 106, wherein the inducible promoter is a light inducible promoter, a nitrate inducible promoter, or a heat responsive promoter. 110. The vector of claim 106, wherein the promoter is T7, psbD, psdA, tufA, ItrA, atpA, or tubulin. 111. The vector of claim 106, wherein the promoter is a chloroplast promoter. 112. The vector of claim 106, wherein the promoter is psbA, psbD, atpA, or tufA. 113. The vector of any one of claims 106 to 112, wherein the promoter is operably linked to the polynucleotide. 114. The vector of claim 91, wherein said vector further comprises a 5′ regulatory region. 115. The vector of claim 114, wherein said 5′ regulatory region further comprises a promoter. 116. The vector of claim 115, wherein said promoter is a constitutive promoter. 117. The vector of claim 115, wherein said promoter is an inducible promoter. 118. The vector of claim 117, wherein said inducible promoter is a light inducible promoter, nitrate inducible promoter, or a heat responsive promoter. 119. The vector of any one of claims 114 to 118, further comprising a 3′ regulatory region. 120. The vector of any one of claims 115 to 118, wherein the promoter is operably linked to the polynucleotide. 121. The vector of any one of claims 91 to 120, wherein the polynucleotide further comprises a nucleic acid which facilitates homologous recombination into a genome of the photosynthetic bacterium, yeast, alga, or vascular plant. 122. The vector of claim 121, wherein the genome is a chloroplast genome of the alga or the vascular plant. 123. The vector of claim 121, wherein the genome is a nuclear genome of the yeast, the alga, or the vascular plant. 124. The vector of claim 91, wherein the photosynthetic bacterium is a member of genera Synechocystis, genera Synechococcus, or genera Athrospira. 125. The vector of claim 91, wherein the photosynthetic bacterium is a cyanobacterium. 126. The vector of claim 91, wherein the alga is a microalga. 127. The vector of claim 91, wherein the alga is C. reinhardtii, D. salina, H. pluvalis, S. dimorphus, D. viridis, D. tertiolecta, N. oculata, or N. salina. 128. The vector of claim 91, wherein the alga is a cyanophyta, a prochlorophyta, a rhodophyta, a chlorophyta, a heterokontophyta, a tribophyta, a glaucophyta, a chlorarachniophyte, a euglenophyta, a euglenoid, a haptophyta, a chrysophyta, a cryptophyta, a cryptomonad, a dinophyta, a dinoflagellata, a pyrmnesiophyta, a bacillariophyta, a xanthophyta, a eustigmatophyta, a raphidophyta, a phaeophyta, or a phytoplankton. 129. The vector of claim 91, wherein the polynucleotide further comprises a nucleic acid encoding a tag for purification or detection of the terpene synthase. 130. The vector of claim 129, wherein the tag is a His-6 tag, a FLAG epitope, a c-myc epitope, a Strep-TAGII, a biotin tag, a glutathione S-transferase (GST), a chitin binding protein (CBP), a maltose binding protein (MBP), or a metal affinity tag. 131. The vector of claim 91, wherein the polynucleotide further comprises a nucleic acid encoding an amino acid sequence of SEQ ID NO: 3, SEQ ID NO: 12, SEQ ID NO: 19, SEQ ID NO: 23, or SEQ ID NO: 29. 132. The vector of claim 91, wherein the polynucleotide further comprises a nucleic acid encoding a selectable marker. 133. The vector of claim 74, wherein the selectable marker is kanamycin, chloramphenicol, ampicillin, or glufosinate.

134. A genetically modified organism, comprising a polynucleotide comprising a nucleic acid encoding a terpene synthase, wherein the terpene synthase cyclyzes a terpene, and wherein the terpene synthase is capable of being expressed in the organism, and wherein the organism is a photosynthetic bacterium, a yeast, an alga, or a vascular plant. 135. The genetically modified organism of claim 134, wherein the nucleic acid is codon biased for expression in the photosynthetic bacterium, yeast, alga, or vascular plant. 136. The genetically modified organism of claim 135, wherein the codon bias is hot codon bias. 137. The genetically modified organism of claim 135, wherein the codon bias is regular codon bias. 138. The genetically modified organism of claim 134, wherein the terpene synthase is a diterpene synthase. 139. The genetically modified organism of claim 138, wherein the diterpene synthase is a fusicoccadiene synthase, a kaurene synthase, a casbene synthase, a taxadiene synthase, an abietadiene synthase, or a homolog of any one of the above, 140. The genetically modified organism of claim 139, wherein the diterpene synthase is a fusicoccadiene synthase or a homolog of a fusicoccadiene synthase. 141. The genetically modified organism of claim 134, wherein the nucleic acid comprises a nucleotide sequence of SEQ ID NO: 1, SEQ ID NO:4, SEQ ID NO: 7, SEQ ID NO: 9, SEQ ID NO: 11, SEQ ID NO: 15, SEQ ID NO: 17, SEQ ID NO: 21, SEQ ID NO: 26, SEQ ID NO: 28, SEQ ID NO: 32, SEQ ID NO: 34, SEQ ID NO: 37, SEQ ID NO: 39, SEQ ID NO: 44, SEQ ID NO: 46, SEQ ID NO: 49, SEQ ID NO: 51, SEQ ID NO: 54, or SEQ ID NO: 56. 142. The genetically modified organism of claim 134, wherein the nucleic acid comprises a nucleotide sequence of SEQ ID NO: 4, SEQ ID NO: 7, SEQ ID NO: 11, SEQ ID NO: 17, SEQ ID NO: 21, SEQ ID NO: 28, SEQ ID NO: 34, or SEQ ID NO: 39. 143. The genetically modified organism of claim 134, wherein the nucleic acid encoding a terpene synthase comprises, (a) an amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55; or (b) a homolog of the amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55. 144. The genetically modified organism of claim 143, wherein the homolog has at least 50%, at least 60%, at least 70%, at least 75%, at least 80%, at least 85%, at least 90%, at least 95%, at least 98%, or at least 99% sequence identity to the amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55. 145. The genetically modified organism of claim 134, wherein the terpene synthase comprises an amino acid sequence of SEQ ID NO: 2. 146. The genetically modified organism of claim 134, wherein the nucleic acid comprises a nucleotide sequence of SEQ ID NO: 4 or SEQ ID. NO: 7. 147. The genetically modified organism of claim 134, wherein the nucleic acid comprises the nucleotide sequence of SEQ ID. NO: 7. 148. The genetically modified organism of claim 134, wherein the terpene is a diterpene. 149. The genetically modified organism of claim 148, wherein the diterpene is a cyclical diterpene. 150. The genetically modified organism of claim 134, wherein the terpene is a fusicoccadiene, a casbene, an ent-kaurene, a taxadiene, or an abietadiene. 151. The genetically modified organism of claim 150, wherein the terpene is a fusicoccadiene. 152. The genetically modified organism of claim 151, wherein the fusicoccadiene is fusicocca-2,10(14)-diene. 153. The genetically modified organism of 134, wherein the terpene synthase is a fusion terpene synthase. 154. The genetically modified organism of claim 153, wherein the fusion terpene synthase comprises a portion of a casbene synthase and a portion of a geranylgeranyl-diphosphate (GGPP) synthase. 155. The genetically modified organism of claim 154, wherein the fusion terpene synthase comprises the amino acid sequence of SEQ ID NO: 22. 156. The genetically modified organism of any one of claims 134 to 155, wherein the polynucleotide further comprises a promoter for expression in the photosynthetic bacterium, yeast, alga, or vascular plant. 157. The genetically modified organism of claim 156, wherein the promoter is a constitutive promoter. 158. The genetically modified organism of claim 156, wherein the promoter is an inducible promoter. 159. The genetically modified organism of claim 158, wherein the inducible promoter is a light inducible promoter, a nitrate inducible promoter, or a heat responsive promoter. 160. The genetically modified organism of claim 156, wherein the promoter is T7, psbD, psdA, tufA, ltrA, atpA, or tubulin. 161. The genetically modified organism of claim 156, wherein the promoter is a chloroplast promoter. 162. The genetically modified organism of claim 156, wherein the promoter is psbA, psbD, atpA, or tufA. 163. The genetically modified organism of any one of claims 156 to 162 wherein the promoter is operably linked to the polynucleotide. 164. The genetically modified organism of claim 134, wherein the polynucleotide further comprises a 5′ regulatory region. 165. The genetically modified organism of claim 164, wherein said 5′ regulatory region further comprises a promoter. 166. The genetically modified organism of claim 165, wherein said promoter is a constitutive promoter. 167. The genetically modified organism of claim 165, wherein said promoter is an inducible promoter. 168. The genetically modified organism of claim 167, wherein said inducible promoter is a light inducible promoter, nitrate inducible promoter, or a heat responsive promoter. 169. The genetically modified organism of any one of claims 164 to 168, further comprising a 3′ regulatory region. 170. The genetically modified organism of any one of claims 165 to 168, wherein the promoter is operably linked to the polynucleotide. 171. The genetically modified organism of any one of claim 134-170, wherein the polynucleotide further comprises a nucleic acid which facilitates homologous recombination into a genome of the photosynthetic bacterium, yeast, alga, or vascular plant. 172. The genetically modified organism of claim 171, wherein the genome is a chloroplast genome of the alga or the vascular plant. 173. The genetically modified organism of claim 171, wherein the genome is a nuclear genome of the yeast, the alga, or the vascular plant. 174. The genetically modified organism of claim 134, wherein the photosynthetic bacterium is a member of genera Synechocystis, genera Synechococcus, or genera Athrospira. 175. The genetically modified organism of claim 134, wherein the photosynthetic bacterium is a cyanobacterium. 176. The genetically modified organism of claim 134, wherein the alga is a microalga. 177. The genetically modified organism of claim 134, wherein the alga is C. reinhardtii, D. salina, H. pluvalis, S. dimorphus, D. viridis, D. tertiolecta, N. oculata, or N. salina. 178. The genetically modified organism of claim 134, wherein the alga is a cyanophyta, a prochlorophyta, a rhodophyta, a chlorophyta, a heterokontophyta, a tribophyta, a glaucophyta, a chlorarachniophyte, a euglenophyta, a euglenoid, a haptophyta, a chrysophyta, a cryptophyta, a cryptomonad, a dinophyta, a dinoflagellata, a pyrmnesiophyta, a bacillariophyta, a xanthophyta, a eustigmatophyta, a raphidophyta, a phaeophyta, or a phytoplankton. 179. The genetically modified organism of claim 134, wherein the polynucleotide further comprises a nucleic acid encoding a tag for purification or detection of the terpene synthase. 180. The genetically modified organism of claim 179, wherein the tag is a His-6 tag, a FLAG epitope, a c-myc epitope, a Strep-TAGII, a biotin tag, a glutathione S-transferase (GST), a chitin binding protein (CBP), a maltose binding protein (MBP), or a metal affinity tag. 181. The genetically modified organism of claim 134, wherein the polynucleotide further comprises a nucleic acid encoding an amino acid sequence of SEQ ID NO: 3, SEQ ID NO: 12, SEQ ID NO: 19, SEQ ID NO: 23, or SEQ ID NO: 29. 182. The genetically modified organism of claim 134, wherein the polynucleotide further comprises a nucleic acid encoding a selectable marker. 183. The genetically modified organism of claim 182, wherein the selectable marker is kanamycin, chloramphenicol, ampicillin, or glufosinate. 184. The genetically modified organism of claim 134, wherein the photosynthetic bacterium, yeast, alga, or vascular plant does not normally produce the terpene. 185. The genetically modified organism of claim 134, wherein at least 0.24%, at least 0.5%, at least 0.75%, or at least 1.0% dry weight of the organism is the terpene. 186. The genetically modified organism of claim 134, wherein at least 0.05%, at least 0.1%, at least 0.25%, at least 0.5%, at least 0.75%0, at least 1.0%, at least 1.25%, at least 1.5%, at least 1.75%, at least 2.0%, at least 3.0%, at least 4.0, or at least 5.0% dry weight of the organism is the terpene. 187. The genetically modified organism of claim 134, wherein the genetically modified organism is capable of growing in a high saline environment. 188. The genetically modified organism of claim 187, wherein the organism is alga. 189. The genetically modified organism of claim 188, wherein the alga is D. salina. 190. The genetically modified organism of claim 187, wherein the high saline environment comprises sodium chloride. 191. The genetically modified organism of claim 190, wherein the sodium chloride is about 0.5 to about 4.0 molar sodium chloride.

192. A composition comprising at least 3% terpene and at least a trace amount of a cellular portion of a genetically modified organism.

193. A method of producing a product, comprising: a) transforming an organism with a polynucleotide comprising a nucleic acid encoding a terpene synthase capable of being expressed in the organism, wherein the transformation results in the production or increased production of a terpene, and wherein the organism is a photosynthetic bacterium, a yeast, an alga, or a vascular plant; b) collecting the terpene from the transformed organism; and c) using the terpene to produce a product. 194. The method of claim 193, wherein the nucleic acid is codon biased for expression in the photosynthetic bacterium, yeast, alga, or vascular plant. 195. The method of claim 194, wherein the codon bias is hot codon bias. 196. The method of claim 194, wherein the codon bias is regular codon bias, 197. The method of claim 193, wherein the terpene synthase is a diterpene synthase. 198. The method of claim 197, wherein the diterpene synthase is a fusicoccadiene synthase, a kaurene synthase, a casbene synthase, a taxadiene synthase, an abietadiene synthase, or a homolog of any one of the above. 199. The method of claim 198, wherein the diterpene synthase is a fusicoccadiene synthase or a homolog of a fusicoccadiene synthase. 200. The method of claim 193, wherein the nucleic acid comprises a nucleotide sequence of SEQ ID NO: 1, SEQ ID NO:4, SEQ ID NO: 7, SEQ ID NO: 9, SEQ ID NO: 11, SEQ ID NO: 15, SEQ ID NO: 17, SEQ ID NO: 21, SEQ ID NO: 26, SEQ ID NO: 28, SEQ ID NO: 32, SEQ ID NO: 34, SEQ ID NO: 37, SEQ ID NO: 39, SEQ ID NO: 44, SEQ ID NO: 46, SEQ ID NO: 49, SEQ ID NO: 51, SEQ ID NO: 54, or SEQ ID NO: 56. 201. The method of claim 193, wherein the nucleic acid comprises a nucleotide sequence of SEQ ID NO: 4, SEQ ID NO: 7, SEQ ID NO: 11, SEQ ID NO: 17, SEQ ID NO: 21, SEQ ID NO: 28, SEQ ID NO: 34, or SEQ ID NO: 39. 202. The method of claim 193, wherein the nucleic acid encoding a terpene synthase comprises, (a) an amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55; or (b) a homolog of the amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55. 203. The method of claim 202, wherein the homolog has at least 50%, at least 60%, at least 70% at least 75%, at least 80%, at least 85%, at least 90%, at least 95%, at least 98%, or at least 99% sequence identity to the amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55, 204. The method of claim 193, wherein the terpene synthase comprises an amino acid sequence of SEQ ID NO: 2. 205. The method of claim 193, wherein the nucleic acid comprises a nucleotide sequence of SEQ ID. NO: 4 or SEQ ID. NO: 7. 206. The method of claim 193, wherein the nucleic acid comprises the nucleotide sequence of SEQ ID. NO: 7. 207. The method of claim 193, wherein the terpene is a diterpene. 208. The method of claim 207, wherein the diterpene is a cyclical diterpene. 209. The method of claim 193, wherein the terpene is a fusicoccadiene, a casbene, an ent-kaurene, a taxadiene, or an abietadiene. 210. The method of claim 209, wherein the terpene is a fusicoccadiene. 211. The method of claim 210, wherein the fusicoccadiene is fusicocca-2,10(14)-diene. 212. The method of claim 193, wherein the terpene synthase is a fusion terpene synthase. 213. The method of claim 212, wherein the fusion terpene synthase comprises a portion of a casbene synthase and a portion of a geranylgeranyl-diphosphate (GGPP) synthase. 214. The method of claim 213, wherein the fusion terpene synthase comprises the amino acid sequence of SEQ ID NO: 22. 215. The method of any one of claims 193 to 214, wherein the polynucleotide further comprises a promoter for expression in the photosynthetic bacterium, yeast, alga, or vascular plant. 216. The method of claim 215, wherein the promoter is a constitutive promoter. 217. The method of claim 215, wherein the promoter is an inducible promoter. 218. The method of claim 217, wherein the inducible promoter is a light inducible promoter, a nitrate inducible promoter, or a heat responsive promoter. 219. The method of claim 215, wherein the promoter is T7, psbD, psdA, tufA, ltrA, atpA, or tubulin. 220. The method of claim 215, wherein the promoter is a chloroplast promoter. 221. The method of claim 215, wherein the promoter is psbA, psbD, atpA, or tufA, 222. The method of any one of claims 215 to 221, wherein the promoter is operably linked to the polynucleotide. 223. The method of claim 193, wherein the polynucleotide further comprises a 5′ regulatory region. 224. The method of claim 223, wherein said 5′ regulatory region further comprises a promoter. 225. The method of claim 224, wherein said promoter is a constitutive promoter. 226. The method of claim 224, wherein said promoter is an inducible promoter. 227. The method of claim 226, wherein said inducible promoter is a light inducible promoter, nitrate inducible promoter, or a heat responsive promoter. 228. The method of any one of claims 223 to 227, further comprising a 3′ regulatory region. 229. The method of any one of claims 224 to 227, wherein the promoter is operably linked to the polynucleotide. 230. The method of any one of claims 193 to 229, wherein the polynucleotide further comprises a nucleic acid which facilitates homologous recombination into a genome of the photosynthetic bacterium, yeast, alga, or vascular plant. 231. The method of claim 230, wherein the genome is a chloroplast genome of the alga or the vascular plant. 232. The method of claim 230, wherein the genome is a nuclear genome of the yeast, the alga, or the vascular plant. 233. The method of claim 193, wherein the photosynthetic bacterium is a member of genera Synechocystis, genera Synechococcus, or genera Athrospira. 234. The method of claim 193, wherein the photosynthetic bacterium is a cyanobacterium. 235. The method of claim 193, wherein the alga is a microalga. 236. The method of claim 193, wherein the alga is C. reinhardtii, D. salina, H. pluvalis, S. dimorphus, D. viridis, D. tertiolecta, N. oculata, or N. salina. 237. The method of claim 193, wherein the alga is a cyanophyta, a prochlorophyta, a rhodophyta, a chlorophyta, a heterokontophyta, a tribophyta, a glaucophyta, a chlorarachniophyte, a euglenophyta, a euglenoid, a haptophyta, a chrysophyta, a cryptophyta, a cryptomonad, a dinophyta, a dinoflagellata, a pyrmnesiophyta, a bacillariophyta, a xanthophyta, a eustigmatophyta, a raphidophyta, a phaeophyta, or a phytoplankton. 238. The method of claim 193, wherein the polynucleotide further comprises a nucleic acid encoding a tag for purification or detection of the terpene synthase. 239. The method of claim 238, wherein the tag is a His-6 tag, a FLAG epitope, a c-myc epitope, a Strep-TAGII, a biotin tag, a glutathione S-transferase (GST), a chitin binding protein (CBP), a maltose binding protein (M3BP), or a metal affinity tag. 240. The method of claim 193, wherein the polynucleotide further comprises a nucleic acid encoding an amino acid sequence of SEQ ID NO: 3, SEQ ID NO: 12, SEQ ID NO: 19, SEQ ID NO: 23, or SEQ 11) NO: 29. 241. The method of claim 193, wherein the polynucleotide further comprises a nucleic acid encoding a selectable marker. 242. The method of claim 241, wherein the selectable marker is kanamycin, chloramphenicol, ampicillin, or glufosinate. 243. The method of claim 193, wherein the photosynthetic bacterium, yeast, alga, or vascular plant does not normally produce the terpene. 244. The method of any one of claims 193-243, further comprising growing the organism in an aqueous environment. 245. The method of claim 244, wherein the growing comprises supplying CO₂ to the organism. 246. The method of claim 245, wherein the CO₂ is at least partially derived from a burned fossil fuel. 247. The method of claim 245 wherein the CO₂ is at least partially derived from flue gas. 248. The method of any one of claims 193 to 247, wherein the collecting step comprises one or more of the following steps: (a) harvesting the transformed organism; (b) harvesting the terpene from a medium comprising the transformed organism; (c) mechanically disrupting the transformed organism; or (d) chemically disrupting the transformed organism.

Methods and compositions described herein utilize terpene/terpenoid synthases, such as fusicoccadiene synthase, for the production of terpenes and terpenoids, including fusicoccadiene, in various organisms. Methods are provided to create organisms genetically modified to produce terpenes and terpenoids. Production of terpenes and terpenoids or their derivatives are useful source of hydrocarbons which can be a source material for the production of fuel. Methods are provided by which terpene synthases, for example PaFS, are engineered to be expressed in genetically modified host cells, for example, cyanobacteria, yeast and algae, where the synthase(s) result in the production or increased production of terpenes and terpenoids, such as fusicoccadiene. In some instances, the terpenes and terpenoids are metabolically inactive in the host cell, leading to a build up of hydrocarbons. Such build up of hydrocarbons increases the usefulness of the engineered host cells for the purpose of fuel production. In some instances, the hydrocarbons can be secreted from the host cell, either naturally or by introduction of a terpene/terpenoid secretion protein,

Described herein is a vector comprising a nucleic acid encoding a terpene synthase, wherein the terpene synthase both condenses and/or cyclyzes a terpene and wherein the nucleic acid is codon biased for expression in photosynthetic bacteria, yeast, algae or vascular plant. A vector described herein can contain a nucleic acid in which one or more codons are biased toward the usage of a target organism. Of various methods available for introducing codon bias to a gene, vectors described herein can contain a codon bias that is known as “hot” codon bias. In some instances, a vector encodes a terpene synthase wherein the terpene synthase is fusicoccadiene synthase or a homolog thereof. In some instances, the homolog has at least 50%, at least 60%, at least 70%, at least 75%, at least 80%, at least 85%, at least 90%, at least 95%, at least 98%, or at least 99% sequence identity to the amino acid sequence of SEQ ID. NO: 2. Alternatively, a vector can comprise a nucleic acid sequence, such as SEQ ID. NO: 4 or SEQ ID. NO: 7, both of which encode for a fusicoccadiene synthase. In some instances, vectors described herein further comprise a promoter for expression in photosynthetic bacteria, non-photosynthetic bacteria, yeast or algae. A vector can utilize promoter sequences derived from, for example, T7 (bacteriophage T7), tD2 (truncated tD2 promoter of Chlamydomonas), D1 (Chlamydomonas), psbD (Scenedesmus) or tufA (Scenedesmus). Other types of promoters contemplated in the present disclosure include promoters driving gene expression in a chloroplast or a nucleus of a host organism. A vector can include nucleic acid sequences which facilitate homologous recombination in a genome of an organism, such as a nuclear genome or a chloroplast genome, especially a microalgal chloroplast genome. Microalgal host organisms which can be transformed with the vectors of the present disclosure include Chlamydomonas reinhardtii, Dunaliella salina, Haematococcus pluvalis, Scenedesmus dimorphus, D. viridis, or D. tertiolecta.

Also described herein is a genetically modified organism comprising an endogenous or exogenous nucleic acid encoding an enzyme, wherein the enzyme both condenses and/or cyclyzes a terpene. Depending on the specific gene introduced, the enzyme may have chain elongation activity, cyclization activity, or both chain elongation and cyclization activities. Organisms useful for the present disclosure include a photosynthetic bacterium, non-photosynthetic bacterium, yeast or alga. An example of the photosynthetic bacterium is a cyanobacterium, such as Synechocystis, Synechococcus, or Athrospira. Non-limiting examples of algal organisms are C. reinhardtii, D. salina, H. pluvalis, S. dimorphus, D. viridis, and D. tertiolecta. Genetically modified organisms disclosed herein can produce one or more terpene synthases. A terpene synthase can be a fusicoccadiene synthase. One of the products that may be produced in the genetically modified organism is fusicoccadiene, for example, fusicocca-2,10(14)-diene. In some instances, the fusicoccadiene is metabolically inactive in the genetically modified organism.

A genetically modified organism of the present disclosure can be a photosynthetic bacterium wherein the bacterium contains at least 0.25%, at least 0.5%, at least 0.75% or at least 1.0% dry weight as a fusicoccadiene. A genetically modified organism can also be an alga wherein the alga contains at least 0.05%, at least 0.1%, at least 0.25%, at least 0.5%, at least 0.75%, at least 1.0%, at least 1.25%, at least 1.5%, at least 1.75%, at least 2.0%, at least 3.0%, at least 4.0% or at least 5.0% dry weight as fusicoccadiene. Exogenous or endogenous nucleic acids described herein can be present in the chloroplast and/or nucleus of an organism. In one embodiment, one or more nucleic acids are integrated into a genome of the chloroplast. In another embodiment, the chloroplast is homoplasmic for the nucleic acid. In some instances, genetic modification of a host cell results in the host cell comprising sufficient chlorophyll levels for the organism to be photoautotrophic. Examples of the organisms useful for genetic modification described herein include cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, and phytoplankton.

Some methods and compositions described herein are directed to a vector comprising a nucleic acid encoding an enzyme capable of modulating a fusicoccadiene biosynthetic pathway. Such a vector may further comprise a promoter for expression of the nucleic acid in bacteria, yeast or algae. Nucleic acid(s) included in such vectors may contain a codon biased form of a gene, optimized for expression in a host organism of choice. Such organisms can be a photosynthetic, a unicellular and/or eukaryotic. In some instances, vectors described herein further comprise a nucleic acid encoding a tag for purification or detection of an enzyme, and a nucleic acid sequence for homologous recombination into a genome of a host cell. In some instances, the target genome is a chloroplast genome. In other instances, the target genome is a nuclear genome. In one embodiment, the fusicoccadiene produced is fusicocca-2,10(14)-diene.

Another aspect of the present disclosure is directed to a vector comprising a nucleic acid encoding an enzyme that produces a fusicoccadiene when the vector is integrated into a genome of an organism, such as photosynthetic bacteria, yeast or algae, wherein the organism does not produce fusicoccadiene without the vector and wherein the fusicoccadiene is metabolically inactive in the organism. In some instances, each codon of the nucleic acid encoding the enzyme which is not a preferred codon of the organism is codon biased. A vector of the present disclosure can utilize “hot” codon bias or “regular” codon bias. A vector encoding an enzyme such as fuisicoccadiene synthase or a homolog thereof may be modified by “hot” codon bias. A homolog useful in the present disclosure may have at least 50%, at least 60%, at least 70%, at least 75%, at least 80%, at least 85%, at least 90%, at least 95%, at least 98%, or at least 99% sequence identity to, for example, the amino acid sequence of SEQ ID. NO: 2. In another embodiment, a nucleic acid encoding an enzyme that produces fusicoccadiene can be a nucleic acid sequence disclosed herein, such as SEQ ID. NO: 4 or SEQ ID. NO: 7. In some instances, a vector of the present disclosure may further comprise a promoter for expression in photosynthetic bacteria, yeast or algae, for example, a vector may include a T7, psaD, tubulin, tD2, D1, psbD or tufA promoter. In other instances, a promoter on a vector of the present disclosure may be a chloroplast promoter, such as tD2, D1, psbD, or tufA. A vector can also include nucleic acid sequences known to facilitate homologous recombination in a genome of an organism, such as a chloroplast genome, especially a microalga 1 chloroplast genome. Sequences for homologous recombination can include sequences from a chloroplast genome of C. reinhardtii, D. salina, H. pluvalis, S. dimorphus, D. viridis, or D. tertiolecta.

Also provided herein are genetically modified chloroplasts comprising any of the vectors of the present disclosure. Additionally, non-vascular, photosynthetic organisms which comprise genetically modified chloroplasts of the present disclosure are disclosed. In some instances, a non-vascular organism is an alga, including microalgae, such as C. reinhardtii, D. salina, H. pluvalis, S. dimorphus, D. viridis, and D. tertiolecta. In other instances, the non-vascular, photosynthetic organisms can be a photosynthetic bacterium, such as a member of the genera Synechocystis, Synechococcus, or Athrospira.

Further described herein are genetically modified, non-vascular photosynthetic organisms comprising an exogenous or endogenous nucleic acid encoding an enzyme that modulates a fuisicoccadiene biosynthetic pathway. A genetic modification can lead to the production of a fusicoccadiene that is not naturally produced by the organisms lacking the nucleic acid. In some instances a fusicoccadiene is metabolically inactive in the modified organism. Organisms useful for the present disclosure can be a unicellular organism, such as a cyanobacterium, yeast or alga. In some instances an exogenous nucleic acid encoding an enzyme is one that is specifically disclosed herein, such as SEQ ID NO: 44 and SEQ ID NO:46 (a nucleic acid sequence encoding the protein EAS27885 from Coccidioides immitis), SEQ ID NO: 49 and SEQ ID NO:51 (a nucleic acid sequence encoding the protein EAA68264 from Gibberella zeae), SEQ ID NO: 54 and SEQ ID NO:56 (a nucleic acid sequence encoding the protein ACLA 076850 from Aspergillus clavatus), or the nucleic acid sequence of SEQ ID NO: 4, or the nucleic acid sequence of SEQ ID NO: 7.

Further provided herein is a method of producing a fuel product, comprising: a) transforming an organism, wherein the transformation results in the production or increased production of a fusicoccadiene; b) collecting the fusicoccadiene from the organism; and c) using the fusicoccadiene to produce a fuel product. In some instances, the organism is an alga, including microalgae such as e C. reinhardtii, D. salina, H. pluvalis, S. dimorphus, D. viridis, and D. tertiolecta. In another embodiment, the organism can be a photosynthetic bacterium, such as a member of the genera Synechocystis, Synechococcus, or Athrospira. In still other embodiments, the organism can be a non-photosynthetic bacterium or yeast. In some aspects, a method provided herein further comprises growing the organism in an aqueous environment, wherein CO₂ is supplied to the organism. The CO₂ can be at least partially derived from a burned fossil fuel or flue gas. In some embodiments, the collecting step of the method comprises one or more of the following steps: (a) harvesting the transformed organism; (b) harvesting the diterpene from a cell medium; (c) mechanically disrupting the organism; or (d) chemically disrupting the organism.

Methods and compositions described herein are directed to a fuel product comprising a hydrocarbon refined from a fusicoccadiene. In some instances, the fusicoccadiene is obtained from a microorganism, such bacteria, yeast, or algae. Such microorganisms can be photosynthetic. In one embodiment, the fusicoccadiene is fusicocca-2,10(14) diene. A fuel product may further comprise a fuel additive,

A method for identifying diterpene synthases with a desired trait is also described herein. In some instances, such a method comprises the steps of: a) performing one or more genetic manipulations on a nucleic acid encoding a diterpene synthase to produce a modified diterpene synthase; b) transforming the modified diterpene synthase into a microorganism; c) growing the microorganism to produce a diterpene; d) analyzing the diterpene; and e) identifying the transformed microorganism having the desired trait. Examples of a desired trait are the expression level of the diterpene synthase, the production level of the diterpene, or the species of diterpene produced. Genetic manipulations utilized in the method include look-through mutagenesis or walk-through mutagenesis. In some instances, the organism is an alga, including microalgae such as e C. reinhardtii, D. salina, H. pluvalis, S. dimorphus, D. viridis, and D. tertiolecta. In another embodiment, the organism can be a photosynthetic bacterium, such as a member of the genera Synechocystis, Synechococcus, or Athrospira. A diterpene produced by a method disclosed herein can be cyclical, such as fusicoccadiene.

Another aspect disclosed herein is a genetically modified organism comprising a nucleic acid encoding a diterpene synthase wherein the organism can grow in a high saline environment. In one embodiment, the organism is a non-vascular, photosynthetic organism, for example D. salina. A high saline environment in some embodiments comprises 0.5-4.0 molar sodium chloride. A diterpene produced by these organisms can be cyclical, such as fusicoccadiene.

Described herein is a composition comprising at least 3% fusicoccadiene and at least a trace amount of a cellular portion of a genetically modified organism. The genetically modified organism can be modified by an exogenous or endogenous nucleic acid encoding fusicoccadiene synthase. In one embodiment, a fuisicoccadiene synthase gene is derived from Phomopsis amygdali. An organism for use in the present disclosure can be a bacterium or yeast. In some embodiments the bacterium is a photosynthetic bacterium, such as a member of the genera Synechocystis, Synechococcus, or Athrospira. In other embodiments the organism is an alga, including microalgae, such as C. reinhardtii, D. salina, H. pluvalis, S. dimorphus, D. viridis, and D. tertiolecta.

Further provided herein is a vector comprising: (a) a nucleic acid encoding protein EAS27885 from Coccidioides immitis, protein EAA68264 from Gibberella zeae, or protein EAQ85668 from Chaetomium blobosum, or a homolog thereof: and (b) a promoter configured for expression of the nucleic acid in a host cell. In some instances, the host cell is a bacterium, yeast, or alga. A bacterium useful in some embodiments can be a photosynthetic bacterium, for example, members of the genera Synechocystis, Synechococcus, and Athrospira. Algae useful in some embodiments can be a microalga, such as C. reinhardtii, D. salina, H. pluvalis, S. dimorphus, D. viridis, and D. tertiolecta. A promoter useful for some vectors of the present disclosure is a promoter capable of driving expression in chloroplast. In some instances, a vector further comprises one or more nucleic acids which allow for homologous recombination with a genome of the host cell. In some embodiments, a target genome is a chloroplast genome. Host cells suitable for the vector include cyanophyta, prochlorophyta, rhodophyta, chlorophyta, heterokontophyta, tribophyta, glaucophyta, chlorarachniophytes, englenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, and phytoplankton. A vector disclosed herein may further comprise a nucleic acid encoding a tag for purification or detection of the enzyme and/or a selectable marker.

In some embodiments, a host cell comprising a vector comprising: (a) a nucleic acid encoding protein EAS27885 from Coccidioides immitis, protein EAA68264 from Gibberella zeae, or protein EAQ85668 from Chaetomium blobosum, or a homolog thereof; and (b) a promoter configured for expression of the nucleic acid in a host cell is provided. Host cells can include a bacterium, yeast, or alga. A bacterium can be a photosynthetic bacterium, for example, members of the genera Synechocystis, Synechococcus, and Athrospira. Examples of alga for use in the present disclosure include C. reinhardtii, D. salina, IL pluvalis, S. dimorphus, D. viridis, and D. tertiolecta. In some instances, the vector, or a portion thereof, is present in a chloroplast and can be integrated into a genome of a chloroplast. Where a vector is incorporated into a chloroplast genome, the host cell can be homoplasmic for the vector, or portion thereof.

### BRIEF DESCRIPTION OF THE DRAWINGS

These and other features, aspects, and advantages of the present disclosure will become better understood with regard to the following description, appended claims and accompanying figures where:

FIG. 1 shows the isoprenoid pathway, and exemplary products of the pathway, for example, fusiccoca-2,10(14)-diene.

FIG. 2 shows the MEP pathway for the production of IPP and DMAPP.

FIG. 3 shows an overview of terpene biosynthesis in photosynthetic eukaryotes.

FIG. 4 shows exemplary terpenes biosynthesized by eukaryotes or prokaryotes.

FIGS. 5A, B, and C show the genomic organization of exemplary plant terpenoid synthase genes.

FIGS. 6A, B, and C show mass spectrum analysis containing peaks corresponding to fusicoccadiene and indole produced: in vivo by recombinant fusicoccadiene synthase expressed in E. coli (FIG. 6A); in vitro by isolated recombinant fusicoccadiene synthase expressed in E. coli (FIG. 6B); and in vivo by recombinant fusicoccadiene synthase expressed in C. reinhardtii (FIG. 6C).

FIGS. 7A, B, and C show mass spectrum analysis containing peaks corresponding to fusicoccadiene produced by recombinant fusicoccadiene synthases encoded by genes with different codon biases expressed in C. reinhardtii. FIG. 7A—regular codon bias; FIG. 7B—C. reinhardtii cells lacking the recombinant fusicoccadiene synthase gene; and FIG. 7C—“hot” codon bias.

FIG. 8 shows thin layer chromatogram of algal extracts demonstrating in vivo accumulation of fusicoccadiene.

FIG. 9 shows selection of six transformants of cyanobacterium clones transformed with PaFS.

FIGS. 10A and B show mass spectrum analysis containing peaks corresponding to fusicoccadiene produced by recombinant fusicoccadiene synthase expressed in cyanobacteria (Synechocystis).

FIG. 11 shows an SDS-PAGE gel showing production of fusicoccadiene synthase from a “hot” codon biased gene expressed in bacteria.

FIG. 12 shows a GC/MSD total ion chromatogram analysis containing peaks corresponding to geranylgeraniol produced by a recombinant fusicoccadiene synthase C-terminal prenyltransferase domain expressed in E. coli, along with positive and negative controls.

FIGS. 13A, B, and C show mass spectrum analysis containing peaks corresponding to fusicoccadiene produced by a recombinant fusicoccadiene synthase expressed in cyanobacteria (Synechocystis).

FIGS. 14A and 14B are the total ion chromatogram and mass spectrum, respectively, demonstrating in vivo accumulation of ent-kaurene in Chlamydomonas transformed with recombinant ent-kaurene synthase. FIGS. 14C and 14D are the total ion chromatogram and mass spectrum, respectively, of untransformed Chlamydomonas, demonstrating that there is no accumulation of ent-kaurene.

FIGS. 15A and 15B are the total ion chromatogram and mass spectrum, respectively, demonstrating in vivo accumulation of ent-kaurene in Scenedesmus transformed with recombinant ent-kaurene synthase. FIG. 15C is the total ion chromatogram of untransformed Scenedesmus, demonstrating that there is no accumulation of ent-kaurene.

FIG. 16 shows plant expression vector pEarleyGate104.

FIGS. 17A and 17B are the total ion chromatogram and mass spectrum, respectively, demonstrating in vivo accumulation of casbene in Chlamydomonas transformed with a recombinant fusion synthase.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present disclosure.

As used in this specification and the appended claims, the singular forms “a”, “an” and “the” include plural reference unless the context clearly dictates otherwise.

Endogenous

An endogenous nucleic acid, nucleotide, polypeptide, or protein as described herein is defined in relationship to the host organism. An endogenous nucleic acid, nucleotide, polypeptide, or protein is one that naturally occurs in the host organism.

Exogenous

An exogenous nucleic acid, nucleotide, polypeptide, or protein as described herein is defined in relationship to the host organism. An exogenous nucleic acid, nucleotide, polypeptide, or protein is one that does not naturally occur in the host organism or is a different location in the host organism.

Isoprenes and Isoprenoids

Over 55,000 individual isoprenoid compounds have been characterized, and hundreds of new structures are reported each year. Most of the molecular diversity in the isoprenoid pathway is created from the disphosphate esters of simple linear polyunsaturated allylic alcohols such as dimethyl alcohol (a 5-carbon molecule), geranoil (a 10-carbon molecule), farnesol (a 15-carbon molecule), and geranylgeraniol (a 20-carbon molecule). The hydrocarbon chains are constructed one isoprene unit at a time by addition of the allylic moiety to the double bond in isopentenyl diphosphate, the fundamental five-carbon building block in the pathway, to form the next higher member of the series. Geranyl, farnesyl, and geranylgeranyl diphosphate lie at multiple branch points in the isoprenoid pathway and are substrates for many enzymes. These are primary cyclases, which are responsible for generating the diverse carbon skeletons for the synthesis of the thousands of mono-, sequi-, di-, and triterpenes; sterols; and carotenoids found in nature. The structures of several of these cyclases have been reported (Lesburg, C. A., et al., Science, Vol. 277, 1820 (1997); Wendt, K. U., et al., Science, Vol. 277, 1811 (1997); and Starks, C. M., et al., Science, Vol. 277, 1815 (1997)).

The extensive family of isoprenoid compounds is synthesized from two-precursors, isopentenyl diphosphate and dimethylallyl disphosphate. The chain elongation and cyclization reactions of isoprenoid metabolism are electrophillic alkylations in which a new carbon-carbon single bond is formed by attaching a highly reactive electron-deficient carbocation to an electron-rich carbon-carbon double bond. From a chemical viewpoint, the most difficult step is generation of the carbocations. Nature has selected three strategies for catalysis: cleavage of the carbon-oxygen bond in an allylic disphosphate ester; protonation of a carbon-carbon double bond, or protonation of an epoxide. Once formed, the carbocations can rearrange by hydrogen atom or alkyl group shifts and subsequently cyclize by alkylating nearby double bonds. Diverse families of isoprenoid structures, often formed from the same substrate in and enzyme-specific manner, are thought to arise from differences in (i) the way substrate is folded in the active site, (ii) how carbocationic intermediates are stabilized to encourage or discourage rearrangements, and (iii) how positive charge is quenched when the product is formed.

Several of the enzymes involved in isoprenoid chain elongation and cyclization have been studied and genetic information is available for some of the enzymes. Although there is little overall similarity between amino acid sequences for the chain elongation and cyclization enzymes, proteins from both classes that use allylic diphosphates as substrates contain highly conserved aspartate-rich DDXXD motifs (D is aspartate, X is any amino acid) thought to be Mg2+ binding sites.

The cyclase domains of the three isoprenoid cyclases as well as farnesyl diphosphate synthase have a similar structural motif, consisting of 10 to 12 mostly antiparallel, alpha helices that form a large active site cavity (as described in Tarshis, L.C., Biochemistry, 33, 10871 (1994)). Lesburg, C. A., et al. (Science, Vol. 277, 1820 (1997)) have labeled this motif the “isoprenoid synthase fold.” In addition, aspartate-rich clusters are present in all four proteins. Three enzymes that use disphosphate-containing substrates (pentalenene synthase, epi-aristolochene synthase, and farnesyl disphosphate synthase) all contain DDXXD on the walls of their active site cavity (for example, as described in Sacchettini, J.C., and Poulter, C. D, Science, Vol. 277, no. 5333, pp. 1788-1789 (1997)). The aspartates are involved in binding multiple Mg2+ ions. The amino acid sequence of hopene synthase also contains a DDXXD motif. Pentalenene synthase and epi-aristolochene synthase also catalyze proton-promoted cyclizations (as described in for example, Sacchettini, J. C., and Poulter, C. D, Science, Vol. 277, no. 5333, pp. 1788-1789 (1997); and Starks, C. M., et al., Science, Vol. 277, 1815 (1997)).

### Terpenes and Terpenoids

Liquid fuels (gasoline, diesel, jet fuel, kerosene, etc) are primarily composed of mixtures of paraffinic and aromatic hydrocarbons. Terpenes are a class of biologically produced molecules synthesized from five carbon precursor molecules in a variety of organisms. Terpenes are pure hydrocarbons, while terpenoids may contain one or more oxygen atoms. Because they are hydrocarbons with a low oxygen content and contain no nitrogen or other heteroatoms, terpenes can be used as fuel components with minimal processing (as described, for example, in Calvin, M. (2008) “Fuel oils from euphorbs and other plants” Botanical Journal of the Linnean Society 94:97-110, and U.S. Pat. No. 7,037,348).

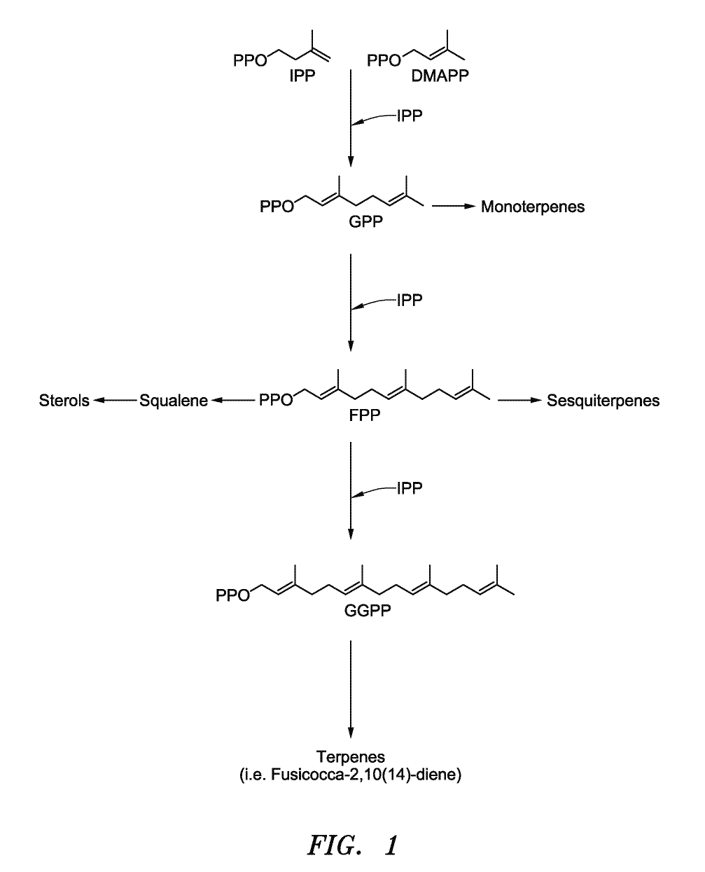

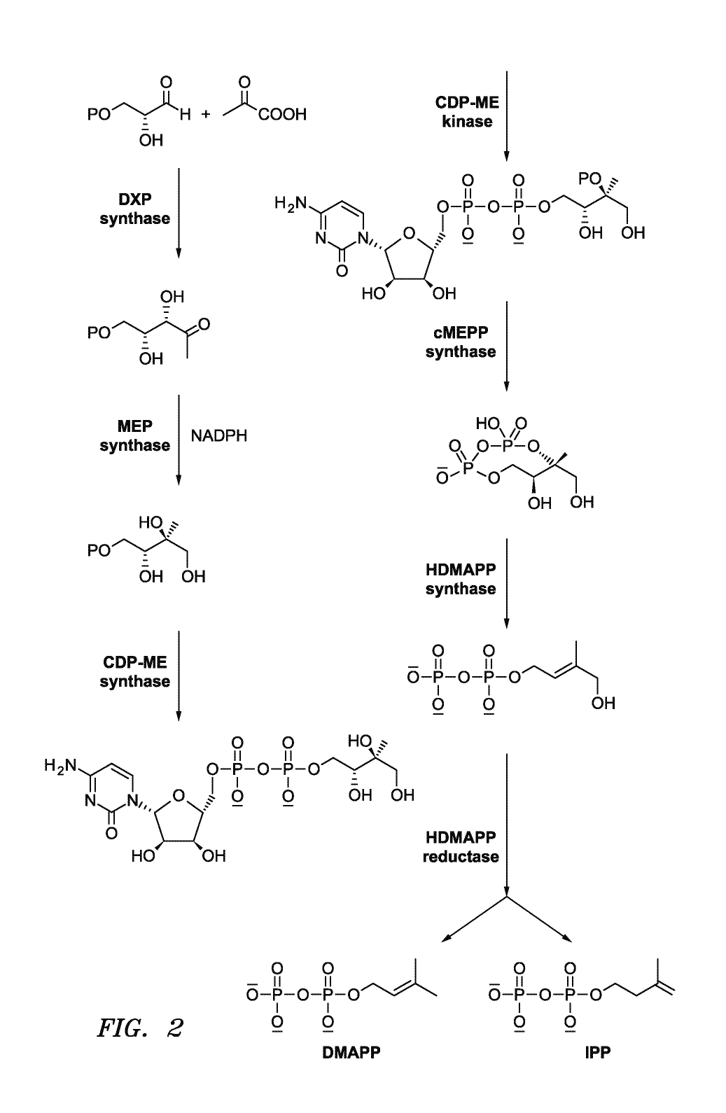

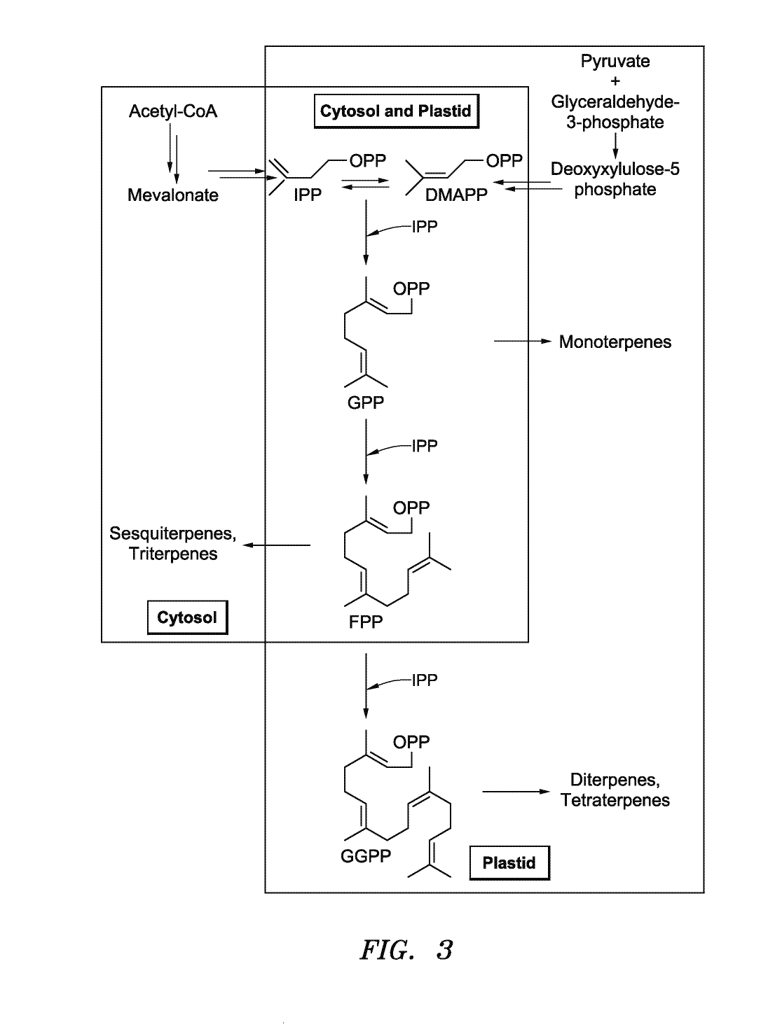

Terpenes are a subset of isoprenes. Terpenes are synthesized in biological systems from two five-carbon precursor molecules, isopentyl-diphosphate and dimethylallyldiphosphate (see FIG. 2). The five-carbon precursors are produced through two pathways, the MEP and the mevalonic acid pathways (see FIG. 2 and FIG. 3). Through condensation reactions, the ten-, fifteen-, and twenty-precursor molecules geranyl diphosphate, farnesyl diphosphate, and geranylgeranyl diphosphate are produced by chain elongation enzymes. These terpenoids are then cyclyzed by terpene synthases into monoterpenes (C10 molecules), sesquiterpenes (C15 molecules), and diterpenes (C20 molecules). Farnesyl diphosphate can be condensed into C30 terpenes, and geranylgeranyl diphosphate can be condensed into C20, C40, or higher molecular weight terpenes. FIG. 1 and FIG. 3 provide an overview of terpenoid biosynthesis.

An overview of terpene biosynthesis in photosynthetic eukaryotes is shown in FIG. 3. The intracellular compartmentalization of the mevalonate and mevalonate-independent pathways for the production of isopentenyl diphosphate (IPP) and dimethylallyl diphosphate (DMAPP), and of the derived terpenoids, is illustrated. The cytosolic pool of IPP, which serves as a precursor of farnesyl diphosphate (FPP) and, ultimately, the sesquiterpenes and triterpenes, is derived from mevalonic acid (left). The plastidial pool of IPP is derived from the glycolytic intermediates pyruvate and glyceraldehyde-3-phosphate and provides the precursor of geranyl diphosphate (GPP) and geranylgeranyl disphosphate (GGPP) and, ultimately, the monoterpenes, diterpenes, and tetraterpenes (right). Reactions common to both pathways are enclosed by both boxes.

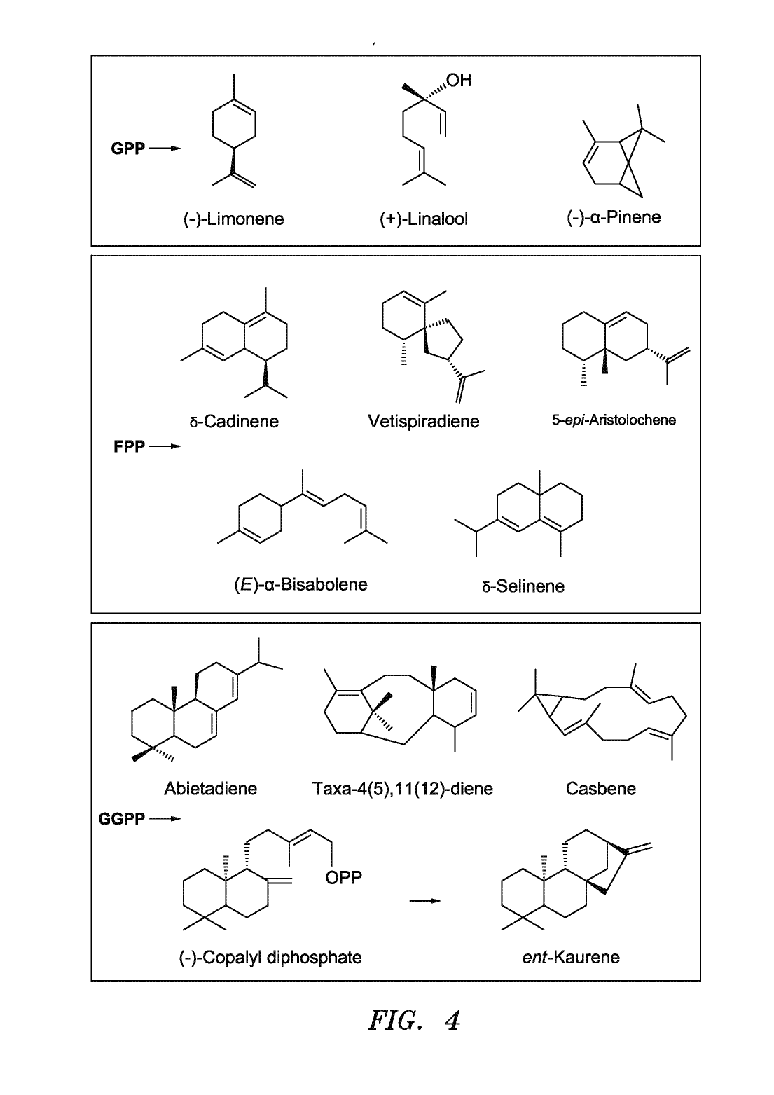

Exemplary terpenes biosynthesized by eukaryotes or prokaryotes are shown in FIG. 4. Monoterpenes, sesquiterpenes, and diterpenes are derived from the prenyl diphosphate substrates, geranyl diphosphate, farnesyl diphosphate, and geranylgeranyl disphosphate, respectively, and are produced in both angiosperms and gymnosperms. (−)-copalyl diphosphate and ent-kaurene are sequential intermediates in the biosynthesis of gibberellins plant growth hormones. Examples of terpenes that can be produced by an organism, for example, an alga, a yeast, a bacteria, or a higher plant, are Casbene, Ent-kaurene, Taxadiene, or Abietadiene (as shown in FIG. 4).

### Fusicoccins and Fusiococcadienes

Fusicoccins or fusiococcadienes are compounds which function in plant pathogenesis and are synthesized by the fungus Phomopsis amygdali. Fusiococcadiene is a cyclic diterpene formed by the condensation of isopentenyl diphosphate (IPP) and dimethylallyl diphosphate (DMAPP) to form the C₂₀ geranylgeranyl diphosphate (GGPP). This linear isoprenoid is then cyclized by a terpene cyclase (fusiococcadiene synthase) to form the tricyclic ring structure of fusiococca-2,10(14)-diene. In P. amygdali, the formation of fusiococca-2,10(14)-diene is carried out by a bifunctional enzyme fusicoccadiene synthase (PaFS), which has both a prenyltransferase domain for the formation of GGPP and a terpene cyclase domain for formation of the tricyclic ring fusicocca-2,10(14)-diene. The carbon skeleton is then modified by oxidation, reduction, methylation, and glycosylation to form fusicoccin A and fusicoccin J, which function to assist plant pathogenesis by permanently activating plant 14-3-3 proteins.

The present description provides methods and compositions for constructing genetically modified organisms which produce terpenes/terpenoids, including cyclical terpenes, such as fusicoccadiene, casbene, ent-kaurene, taxadiene, and abietadiene. Also provided are methods of producing terpenes/terpenoids (such as fusicoccadiene) in genetically modified organisms. In some aspects, the terpenes/terpenoids may be collected from the organism(s) which have been modified to produce them. Collected terpenes/terpenoids may then be further modified, for example by refining and/or cracking to produce fuel molecules or components.

In some instances, a host organism is transformed with a nucleic acid encoding at least one terpene/terpenoid synthase, such as fusicoccadiene synthase. Host organisms can include any suitable host, for example, a microorganism. Microorganisms which are useful for the methods described herein include, for example, photosynthetic bacteria (e.g., cyanobacteria), non-photosynthetic bacteria (e.g., E. coli), yeast (e.g., Saccharomyces cerevisiae), and algae (e.g., microalgae such as Chlamydomonas reinhardtii). Modified organisms are then grown, in some embodiments in the presence of CO₂, to produce the terpene/terpenoid. In one embodiment, the terpene/terpenoid is fusicoccene.

Methods and compositions described herein may take advantage of naturally occurring product production pathways in an organism, for example, a photosynthetic organism. An example of one such production pathway is the isoprenoid biosynthetic pathway. Methods and compositions described herein may take advantage of naturally occurring biological molecules as substrates for the recombinantly expressed enzyme or enzymes of interest. IPP, DMAPP, FPP, and GPP may serve as substrates for enzymes of the present disclosure, and may be natively produced in bacteria, yeast, and algae (e.g., through the mevalonate pathway or the MEP pathway (see FIG. 2 and FIG. 3).

Insertion of genes encoding an enzyme of the present disclosure into a host organism may lead to increased production of terpenes/terpenoids and/or derivatives, such as fusicoccadiene. In one disclosed method, fusicocca-2,10(14) diene is produced. Production of terpene/terpenoid derivatives may be artificially increased by introducing extra copies of an artificially engineered, exogenous enzyme modulating the isoprenoid biosynthetic pathway.

Production of fusicoccadiene can be modulated by introducing a fusicoccadiene synthase, such as PaFS, or a homolog derived from bacteria, yeast, fungi, or an animal into an organism. Fusicoccadiene synthase homologs have been identified in Coccidioides immitis, Gibberella zeae, Alternaria brassicicola, and Chaetomiumn blobosum, for example. Production of fusicoccadiene can also be modulated by introducing a portion of PaFS into an organism, wherein the portion exerts an enzymatic activity on a substrate. Enzymes with terpene cyclase activity (terpene synthases) can also be utilized in optimizing the production of a fusicoccadiene. For example, enzymes capable of forming C₂₀ geranylgeranyl diphosphate (GGPP) can be utilized in optimizing the production of a fusicoccadiene.

By way of example, a non-vascular photosynthetic microalga species can be genetically engineered to produce fuisicoccadiene, such as C. reinhardtii, D. salina, H. pluvalis, S. dimorphus, D. viridis, and D. tertiolecta. Production of fusicoccadiene in these microalgae can be achieved by engineering the microalgae to express an exogenous enzyme PaFS in the chloroplast or nucleus. PaFS can convert IPP and DMAPP into fusicocca-2, 10(14)-diene.

The expression of the PaFS can be accomplished by inserting an exogenous gene encoding PaFS into the chloroplast or nuclear genome of the microalgae. The modified strain of microalgae can be made homoplasmic to ensure that the PaFS gene will be stably maintained in the chloroplast genome of all descendents. A microalga is homoplasmic for a gene when the inserted gene is present in all copies of the chloroplast genome, for example. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% or more of the total soluble plant protein. The process of determining the plasmic state of an organism of the present disclosure involves screening transformants for the presence of exogenous nucleic acids and the absence of wild-type nucleic acids at a given locus of interest.

The present disclosure, among other embodiments, provides genetically modified microorganisms capable of producing useful products, for example, terpenes and terpenoids such as fusicoccadiene. In some embodiments, production of a desired terpene/terpenoid is achieved by way of expressing one or more codon biased terpene/terpenoid synthases in the microorganism. Examples of terpene/terpenoid synthases useful for the present disclosure are PaFS or PaFS homologs. Other proteins, such as, for example, EAS27885 from Coccidioides immitis, a nucleic acid encoding protein EAA68264 from Gibberella zeae, or a nucleic acid encoding protein EAQ85668 from Chaetomium blobosum, can be cloned and utilized in the present disclosure. Nucleic acid sequences artificially modified to adopt “regular” codon bias or “hot” codon bias, such as, for example, IS-87 (“regular” codon biased PaFS with a tag; SEQ ID NO: 4) or IS-88 (“hot” codon biased PaFS with a tag; SEQ ID NO: 7) can be utilized in the creation of genetically modified organisms useful for terpene/terpenoid (e.g., fusicoccadiene) production.

### Terpene Synthases

Terpene synthases are also known as terpene cyclases, and these two terms can be used interchangeably throughout the disclosure.

Generally speaking, terpene cyclases use one of three substrates—the ten carbon geranyl diphosphate, fifteen carbon farnesyl diphosphate, or twenty carbon geranylgeranyl diphosphate, as substrates. Cyclases acting on geranyl diphosphate produce ten carbon monoterpenes; those that act on farnesyl diphosphate produce sesquiterpenes, and those that act on geranylgeranyl diphosphate produce diterpenes. Some naturally occurring terpene synthase (for instance, fusicoccadiene synthase from P. amygdali) contain both a terpene cyclase domain, as well as a prenyl transferase or chain elongation domain. If present, this chain elongation domain will produce the GPP, FPP, or GGPP substrate for the cyclase from the five carbon isoprenoids isoprenyl diphosphate and dimethylallyl diphosphate.

In one exemplary organism (Phomopsis amygdali), fusicoccadiene synthase catalyzes two reactions, the first is a prenyl transferase reaction producing GGPP from three molecules of IPP and one molecule of DMAPP, and a second reaction where GGPP is cyclyzed to produce fusicocca-2,10(14)diene and inorganic pyrophosphate. These two reactions reside in two separate domains of the protein; the N-terminal terpene cyclase and the C-terminal prenyl transferase domains.

Terpenoids are the largest, most diverse class of natural products and they play numerous functional roles in primary metabolism, Well over 30 cDNAs encoding plant terpenoid synthases involved in primary and secondary metabolism have been cloned and characterized. Terpenoids are present and abundant in all phyla, and they serve a multitude of functions in their internal environment (primary metabolism) and external environment (ecological interactions). The biosynthetic requirements for terpene production are the same for all organisms (a source of isopentenyl diphosphate, isopentyl diphosphate isomerase or other source of dimethylallyl diphosphate, prenyltransferases, and terpene synthases).

Of the more than 30,000 individual terpenoids now identified (for example, as described in Buckingham, J. (1998) Dictionary of Natural Products on CD-ROM, Version 6.1. Chapman & Hall, London), at least half are synthesized by plants. A relatively small, but quantitatively significant, number of terpenoids are involved in primary plant metabolism including, for example, the phytol side chain of chlorophyll, the carotenoid pigments, the phytosterols of cellular membranes, and the gibberellin plant hormones. However, the vast majority of terpenoids are classified as secondary metabolites, compounds not required for plant growth and development but presumed to have an ecological function in communication or defense (for example as described in Harborne, J. B. (1991) Recent advances in the ecological chemistry of plant terpenoids, pp. 396-426 in Ecologial Chemistry and Biochemistry of Plant Terpenoids, edited by J. B. Harborne and F. A Tomas-Barberan. Clarendon Press, Oxford). Mixtures of terpenoids, such as the aromatic essential oils, turpentines, and resins, form the basis of a range of commercially useful products (for example, as described in Zinkel, D. F. and Russell, J. (1989) Naval Stores: Production, Chemistry, Utilization. Pulp Chemicals Association, New York, p. 1060; and Dawson, F. A. (1994) The Amazing Terpenes. Naval Stores Rev. March/April: 6-12), and several terpenoids are of pharmacological significance, including the monoterpenoid (C10) dietary anticarcinogen limonene (Crowell, P. L. and Gould, M. N. (1994) CRC Crit. Rev. Oncogenesis 5:1-22), the sequiterpenoid (C15) antimalaria artemisin (Van (Van Geldre, E., et al. (1997) Plant Mol. Biol. 33: 199-209), and the diterpenoid anticancer drug Taxol (Holmes, F. A. et al. (1995) Current status of clinical trials with paclitaxel and docetaxel, pp. 31-57 in Taxane Anticancer Agents. Basic Science and Current Status, edited by G. I. George, T. T. Chen, I. Ojima and D. M. Vyas. American Chemical Society Symposium Series 583, Washington D.C.).

All terpenoids are derived from isopentenyl disphosphate (FIG. 2). In plants, this central precursor is synthesized in the cytosol via the classical acetate/mevalonate pathway (for example, as described in Qureshi, N. and Porter, J. W. (1981) Conversion of acetyl-Coenzyme A to isopentenyl pyrophosphate, pp. 47-94 in Biosynthesis of Isoprenoid Compounds, Vol. 1, edited by J. W. Porter and S. L. Spurgeon, John Wiley &. Sons, New York; and Newman, J. D. and Chappell, J. (1999) Crit. Rev. Biochem. Mol. Biol. 34: 95-106), by which the sequiterpenes (C 15) and triterpenes (C30) are formed, and in plastids via the alternative, pyruvate/glyceraldehydes-3-phosphate pathway (for example, as described in Eisenreich, W. M., et al. (1998) Chem. Biol. 5:R221-R233; and Lichtenthaler, H. K. (1999) Annu. Rev. Plant Physiol. Plant Mol. Biol. 50:47-66), by which the monoterpenes (C10), diterpenes (C20), and tetraterpenes (C40) are formed. Following the isomerization of isopentyl disphosphate to dimethylallyl disphosphate, by the action of isopentyl disphosphate isomerase, the latter is condensed with one, two, or three units of isopentenyl disphosphate, by the action of prenyltransferases, to give geranyl disphosphate (C10), farnesyl disphosphate (C15), and geranylgeranyl disphosphate (C20), respectively (for example, as described in Ramos-Valdivia, A. C., et al. (1997) Nat. Prod. Rep. 14:591-603; Ogura, K. and Koyama, T. (1998) Chem. Rev. 98: 1263-1276; Koyama, T. and Ogura, K. (1999) Isopentenyl disphosphate isomerase and prenyltransferases, pp. 69-96 in Comprehensive Natural Products Chemistry Including Steroids and Cartenoids, Vol. 2, edited by I). E. Cane, Pergamon, Oxford; and FIG. 2). These three acyclic prenyl disphosphates serve as the immediate precursors of the corresponding monoterpenoid (C10), sequiterpenoid ((C15), and diterpenoid (C20) classes, to which they are converted by a very large group of enzymes called the terpene (terpenoid) synthases. These enzymes are often referred to as terpene cyclases, since the products of the reactions are most often cyclic.

A large number of terpenoid synthases of the monoterpene (for example, as described in Croteau, R. (1987) Chem. Rev. 87: 929-954; and Wise, M. I. and Croteau, R. (1999) Monoterpene biosynthesis, pp. 97-153 in Comprehensive Natural Products Chemistry: Isoprenoids Including Steroids and Carotenoids, Vol. 2, edited by D. E. Cane, Pergamon, Oxford), sesquiterpene (for example, as described in Cane, D. E, (1990) Isoprenoid biosynthesis: overview, pp. 1-13 in Comprehensive Natural Products Chemistry: Isoprenoids Including Steroids and Cartenoids, Vol. 2, edited by D. E. Cane, Pergamon, Oxford; and Cane, D. E. (1999) Sesquiterpene biosynthesis: cyclization mechanisms, pp. 150-200 in Comprehensive Natural Products Chemistry: Isoprenoids Including Steroids and Cartenoids, Vol. 2, edited by D. E. Cane, Pergamon, Oxford), and diterpene (for example, as described in West, C. A. (1981) Biosynthesis of diterpenes, pp. 375-411 in Biosynthesis of Isoprenoid Compounds, Vol. 1, edited by J. W. Porter and S. L. Spurgeon, John Wiley & Sons, New York; and MacMillan, J. and Beale, M. (1999) Diterpene biosynthesis, pp. 217-243 in Comprehensive Natural Products Chemistry: Isoprenoids Including Steroids and Carotenoids, Vol. 2, edited by D. E. Cane, Pergamon, Oxford) series have been isolated from both plant and microbial sources, and these catalysts have been described in detail. All terpenoid synthases are very similar in physical and chemical properties, for example, in requiring a divalent metal ion as the only cofactor for catalysis, and all operate by electrophilic reaction mechanisms. In this regard, the terpenoid synthases resemble the prenyltransferases; however, it is the tremendous range of possible variations in the carbocationic reactions (cyclizations, hydride shifts, rearrangements, and termination steps) catalyzed by the terpenoid synthases that sets them apart as a unique enzyme class. Indeed, it is these variations on a common mechanistic theme that permit the production of essentially all chemically feasible skeletal types, isomers, and derivatives that form the foundation for the great diversity of terpenoid structures.

Several groups have suggested that plant terpene synthases share a common evolutionary origin based upon their similar reaction mechanism and conserved structural and sequence characteristics, including amino acid sequence homology, conserved sequence motifs, intron number, and exon size (for example, as described in Mau, C. J. D. and West, C., A. (1994) Proc. Natl. Acad. Sci. USA 91: 8479-8501; Back, K. and Chappell, J. (1995). Biol. Chem. 270:7375-7381; Bohlman, J., et al. (1998) Proc. Natl. Acad. Sci. USA 95: 4126-4133; and Cseke, L., et al. (1998) Mol. Biol. Evol. 15: 1491-1498). A sequence comparison between three isolated plant terpenoid synthase genes (a monoterpene cyclase limonene synthase (Colby, S. M., et al. (1993) J. Biol. Chem. 268: 23016-23024), a sesquiterpene cyclase epi-aristolochene synthase (Facchini, P. J. and Chappell, J. (1992) Proc. Natl. Acad. Sci. USA 89:11088-11092), and a diterpene cyclase casbene synthase (Mau, C. D. and West, C. A. (1994) Proc. Natl. Acad. Sci. USA 91: 8479-8501) gave clear indication that these genes, from phylogenetically distant plant species, were related, a conclusion supported by genomic analysis of intron number and location (Mau, C. J. D. and West, C. A. (1994) Proc. Natl. Acad. Sci. USA 91: 8479-8501; Back, K. and Chapell, J. (1995) J. Biol. Chem. 270:7375-7381; Chappell, J. (1995) Plant Physiol. 107:1-6; and Chappell, J. (1995) Annu. Rev. Plant Physiol. Plant Mol. Biol. 46:521-547). Phylogenetic analysis of the deduced amino acid sequences of 33 terpenoid synthases from angiosperms and gymnosperms allowed recognition of six terpenoid synthase (Tps) gene subfamilies on the basis of clades (Bohlmann, J., et al. (1998) Proc. Natl. Acad. Sci. USA 95: 4126-4133). The majority of terpene synthases analyzed produce secondary metabolites and are classified into three subfamilies, Tpsa (sesquiterpene and diterpene synthases from angiosperms), Tpsb (monoterpene synthase from angiosperms of the Lamiaceae), and Tpsd (11 gymnosperm monoterpene, sesquiterpene, and diterpene synthases). The other three subfamilies, Tpsc, Tpse, and Tpsf, are represented by the single angiosperm terpene synthase types copalyl disphosphate synthase, kaurene synthase, and linalool synthase, respectively. The first two are diterpenes synthases involved in early steps of gibberellin biosynthesis (MacMillan, J. and Beale, M. (1999) Diterpene biosynthesis, pp. 217-243 in Comprehensive Natural Products Chemistry: Isoprenoids Including Steroids and Carotenoids, Vol. 2, edited by D. E. Cane, Pergamon, Oxford). These two Tps subfamilies are grouped into a single clade and are involved in primary metabolism, which suggests that the bifurcation of terpenoid synthases of primary and secondary metabolism occurred before the separation of angiosperms and gymnosperms (Bohlmann, J. G., et al. (1998) Proc. Natl. Acad. Sci. USA 95: 4126-4133). A detailed analysis of the monoterpene synthase, linalool synthase from Clarkia representing Tpsf, was conducted by Cseke, L., et al. (1998) Mol. Biol. Evol. 15: 1491-1498.

The isolation and analysis of six genomic clones encoding terpene synthases of conifers, ((−)-pinene (C10), (−)-limonene (C10), (E)-α-bisabolene (C15), 6-selinene (C15), and abietadiene synthase (C20) from Abies grandis and taxadiene synthase (C20) from Taxus brevifolia), all of which are involved in natural products biosynthesis, has been described by Trapp, S. C. and Croteau, R. B., Genetics (2001) 158:811-832. Genome organization (intron number, size, placement and phase, and exon size) of these gymnosperm terpene synthases was compared by Trapp, S. C. and Croteau, R. B. (Genetics (2001) 158:811-832) to eight previously characterized angiosperm terpene synthase genes and to six putative terpene synthase genomic sequences from Arabidopsis thaliana. Three distinct classes of terpene synthase genes were discerned, from which assumed patterns of sequential intron loss and the loss of an unusual internal sequence element suggest that the ancestral terpenoid synthase gene resembled a contemporary conifer diterpene synthase gene in containing at least 12 introns and 13 exons of conserved size.

In addition to gene sequences for several angiosperm terpene synthases being able to be found in public databases, see Table 1, Trapp, S. C. and Croteau, R. B. (Genetics (2001) 158:811-832) determined the genomic sequences of several terpene synthases from gymnosperms. Trapp, S. C. and Croteau, R. B. (Genetics (2001) 158:811-832) determined the genomic (gDNA) sequences corresponding to six (Agggabi, AgfEabis, Agg-pin1, Agfhsel1, Agg-lim, Tbggtax) conifer terpene xynthase cDNAs (Table 1). This selection of genes represents constitutive and inducible terpenoid synthases from each class (monoterpene, sesquiterpene, and diterpene). Sequence alignment of each cDNA with the corresponding gDNA, including putative terpene synthases from Arabidopsis, established exon and intron boundaries, exon and intron sizes, and intron placement; generic dicot plant 5′- and 3′-splice site consensus sequences (5′ NAG▾GTAAGWWWW; and 3′YAG▾) were used to define specific boundaries (Hanley, B. A. and Schuler, M. A. (1988) Nucleic Acid Res. 16:7159-7176; and Turner, G. (1993) Gene organization in filamentous fungi, pp. 107-125 in The Eukaryotic Genome: Organization and Regulation, edited by P. M. A. Borda, S. Oliver, and P. F. G., SIMS, Cambridge University Press, New York). These analyses reveal a distinct pattern of intron phase for each intron throughout the entire Tps gene family.

A wide range of nomenclatures has been applied to the terpenoid synthases, none of which are systematic. Trapp, S. C. and Croteau, R. B. (Genetics (2001) 158:811-832) uses a unified and specific nomenclature system in which the Latin binomial (two letters), substrate (one- to four-letter abbreviation), and product (three letters) are specified. Thus, ag22, the original cDNA designation for abietadiene synthase from A. grandis (a Tpsd subfamily member), becomes AgggABI for the protein and Agggabi for the gene, with the remaining conifer synthases (and other selected genes) described accordingly (for example, as described in Table 1).

A key to Table 1 is provided below.

Tc, genomic sequences by Trapp, S. C. and Croteau, R. B. (Genetics (2001) 158:811-832); NA, sequences unavailable in the public databases but disclosed in journal reference; pc, sequences obtained by personal communications; ds, sequences in public database by direct submission but not published; p, sequences in database with putative function; c, confirmed gene by experimental determination stated in database; i, two possible isozymes reported for the same region referred to as A1 and A2; —, no former gene name or accession number. Species names are: Abies grandis, Arabidopsis thaliana, Clarkia concinna, Gossypium arboreurn, Hyoscyamus muticus, Mentha longifolia, Mentha spicata, Nicotiana tabacum, Ricinus communis, Perilla frutescens, Taxus brevifolia, and Zea mays.

^{a} Former names, respectively, for (2)-copalyl diphosphate synthase and ent-kaurene synthase were ent-kaurene synthase A (KSA) and ent-kaurene synthase B (KSB), and mutant phenotypes were gal and ga2; these designations have been used loosely.

^{b} Nomenclature architecture is specified as follows. The Latin binomial two-letter abbreviations are in spaces 1 and 2. The substrates (1- to 4-letter abbreviations) are in spaces 3-6, consisting of 1- or 2-letter abbreviations for substrate utilized in boldface (e.g., g, geranyl diphosphate; f, farnesyl diphosphate; gg, geranylgeranyl diphosphate; c, copalyl diphosphate; ch, chrysanthemyl diphosphate; in lowercase) followed by stereochemistry and/or isomer definition (e.g., a, b, d, g, etc. followed by epi (e), E, Z, -, i, etc.). The 3-letter product abbreviation indicates the major product is an olefin; otherwise the quenching nucleophile is indicated, (e.g., ABI, abietadiene synthase; BORPP, bornyldiphosphate synthase; CEDOH, cedrol synthase); uppercase specifies protein and lowercase specifies cDNA or gDNA, All letters except species names are in italics for cDNA and gene. Distinction between cDNA and gDNA must be stated or a g is added before the abbreviation, e.g., Tbggtax cDNA and gTbggtax, or Tbggtax gene (nomenclature system devised by S. Trapp, E. Davis, J. Crock, and R. Croteau, and as discussed in Trapp, S. C. and Croteau, R. B., Genetics (2001) 158:811-832).

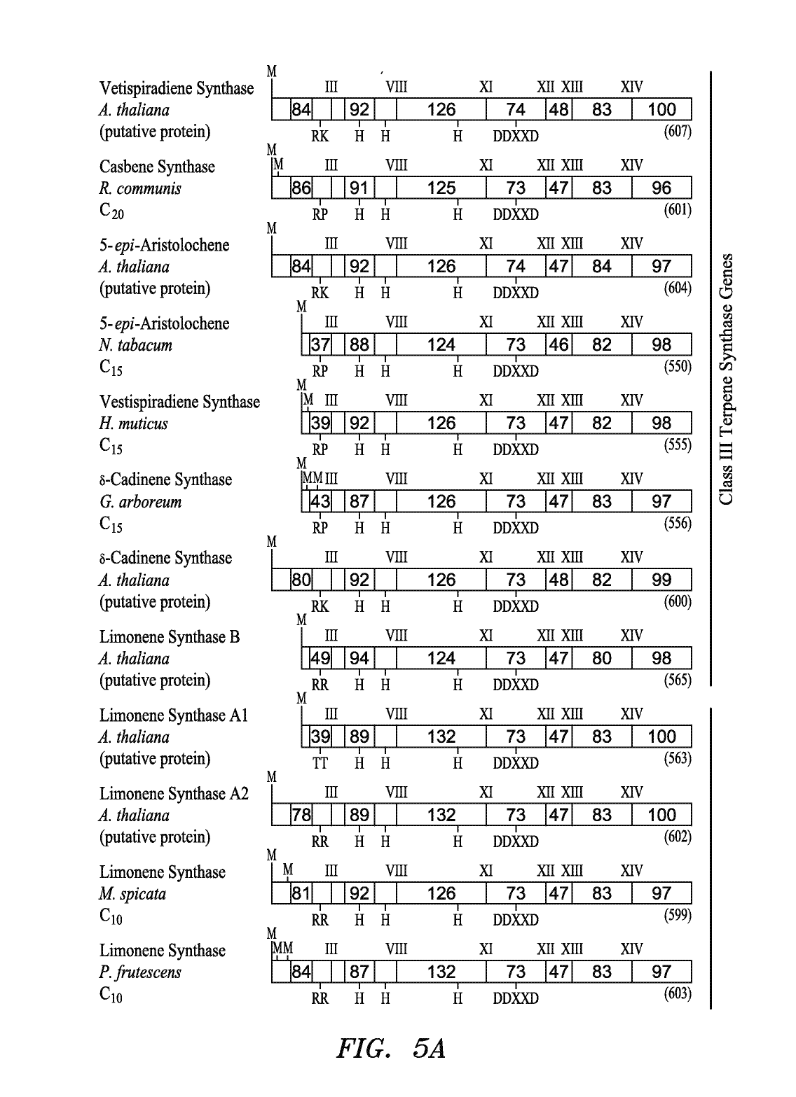

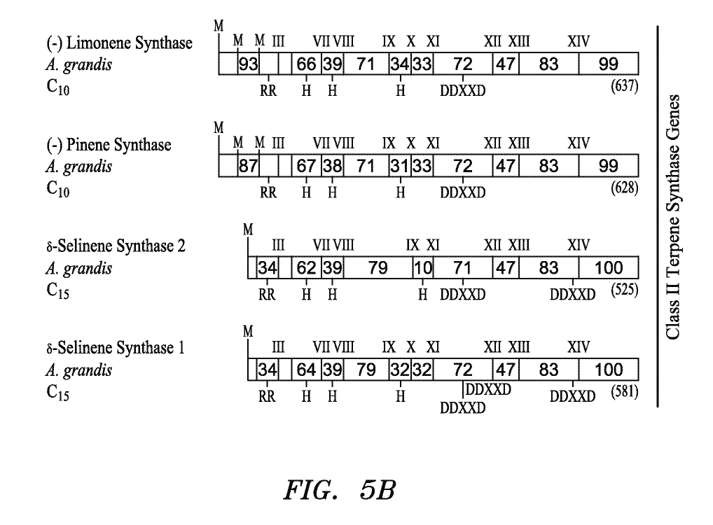

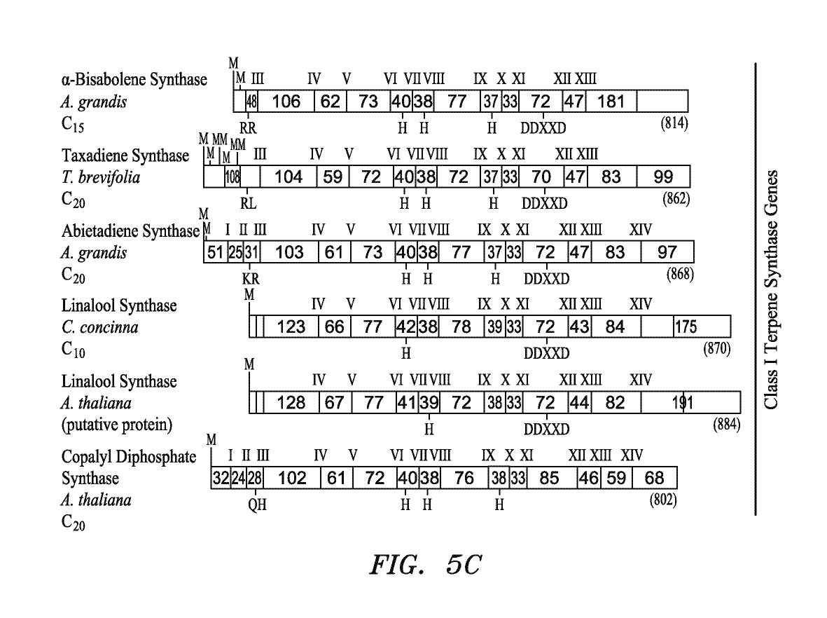

A comparison of genomic structures (as shown in FIGS. 5A, B, and C) indicate that the plant terpene synthase genes consist of three classes based on intron/exon pattern; 12-14 introns (class I), 9 introns (class II), or 6 introns (class III). Using this classification, based on distinctive exon/intron patterns, seven conifer genes that Trapp, S. C. and Croteau, R. B. (Genetics (2001) 158:811-832) studied were assigned to class I or class II. Class I comprises conifer diterpene synthase genes Agggabi and Tbggtax and sesquiterpene synthase Agfabis and angiosperm synthase genes specifically involved in primary metabolism (Atgg-copp1 and Ccglinoh). Terpene synthase class I genes contain 11-14 introns and 12-15 of exons of characteristic size, including the CDIS domain comprising exons 4, 5, and 6 and the first approximately 20 amino acids of exon 7, and introns 4, 5, and 6 (this unusual sequence element corresponds to a 215-amino-acid region (Pro 137- Leu 351) of the Agggabi sequence). Class II Tps genes comprise only conifer monoterpene and sesquiterpene synthases, and these contain 9 introns and 10 exons; introns 1 and 2 and the entire CDIS element have been lost, including introns 4, 5 and 6. Class III Tps genes comprise only angiosperm monoterpene, sesquiterpene, and diterpene synthases involved in secondary metabolism, and they contain 6 introns and 7 exons. Introns 1, 2, 7, 9, and 10, and the CDIS domain have been lost in the class III type. The introns of class III Tps genes (introns 3, 8. and 11-14) are conserved among all plant terpene synthase genes and were described as introns 1-6, respectively, in previous analyses (Mau, C. J. D. and West, C. A. (1994) Proc. Natl. Acad. Sci. USA 91: 8479-8501; Back, K. and Chapell, J. (1995) J. Biol. Chem. 270:7375-7381; and Chappell, J. (1995) Annu. Rev. Plant Physiol. Plant Mol. Biol. 46:521-547).

A number of diterpene products may be produced in vivo by inserting an exogenous or endogenous gene encoding a diterpene synthase into the chloroplast or nuclear genome of an organism, for example, a microalgae, yeast, or plant. When the functional diterpene synthase is expressed by the organism, the exogenous or endogenous enzyme will utilize either the endogenous geranylgeranyl diphosphate as a substrate, or if the exogenous or endogenous enzyme contains a GGPP synthase domain, will utilize the endogenous IPP and DMAPP as substrates. The enzyme will convert the substrates to a diterpene in vivo. Examples of diterpene synthases that may be used in this manner include Abietadiene synthase, Taxadiene synthase, Casbene synthase, and ent-Kaurene synthase.

Trapp, S. C., and Croteau R. B. (Genetics 158:811-832 (2001) studied the genomic organization of plant terpene synthase (Tps) genes and the results of their studies are shown in FIGS. 5A, B, and C. Black vertical bars represent introns 1-14 (Roman numerals in figure) and are separated by shaded blocks with specified lengths, representing exons 1-15. The terpenoid synthase genes are divided into three classes (class I, class II, and class III), which appear to have evolved sequentially from class I to class III by intron loss and loss of the conifer diterpene internal sequence domain (CDIS). (FIG. 5C) Class I Tps genes comprise 12-14 introns and 13-15 exons and consist primarily of diterpene synthases found in gymnosperms (secondary metabolism) and angiosperms (primary metabolism). (FIG. 5B) Class II Tps genes comprise 9 introns and 10 exons and consist of only gymnosperm monoterpene and sesquiterpene synthases involved in secondary metabolism. (FIG. 5A) Class III Tps genes comprise 6 introns and 7 exons and consist of angiosperm monoterpene, sesquiterpene, and diterpene synthases involved in secondary metabolism. Exons that are identically shaded illustrate sequential loss of introns and the CDIS domain, over evolutionary time, from class I through class III. The methionine at the translational start site of the coding region (and alternatives), highly conserved histidines, and single or double arginines indicating the minimum mature protein (Williams, D. C., et al. (1998) Biochemistry 37:12213-12220) are represented by M, H, RR, or RX (X representing other amino acids that are sometimes substituted), respectively. The enzymatic classification as a monoterpene, sesquiterpene, or diterpene synthase is represented by C10, C15, C20, respectively. Conifer terpene synthases were isolated and sequenced to determine genomic structure; all other terpene synthase sequences were obtained from public databases or by personal communication (see Table 1). Putative terpene synthases are referred to as putative proteins and are illustrated based upon predicted homology. Two different predictions of the same putative protein (accession no. Z97341) are shown as limonene synthase A1 and A2; if A1 is correct, the genomic pattern suggests that Atlim (accession no. Z97341) is a sesquiterpene synthase; if A2 is correct, then Atlim (accession no. Z97341) is a monoterpene synthase. In the analysis of intron borders of the Msg-lim/Mlg-lim chimera and Hmfvet1 genes (see Table 1), only a single intron border (5′ or 3′) was sequenced to determine intron placement; size was not determined. The intron/exon borders predicted for a number of terpene synthases identified in the Arabidopsis database were determined to be incorrect; these data were reanalyzed and new predictions used. The number in parentheses represents the deduced size (in amino acid residues) of the corresponding protein or preprotein, as appropriate,

Table 1 provides the names of various terpene synthases and provides the GenBank accession numbers for both the cDNA and gDNA of many of the listed terpene synthases. A listing of the articles cited in Table 1 is provided below.

The following articles are cited in Table 1: Back, K. and Chapell, J. (1995) J. Biol. Chem. 270:7375-7381; Bohlmann, J., et al., (1997) J. Biol. Chem. 272:21784-21792; Bohlmann, J., et al. (1998a) Proc. Natl. Acad. Sci. USA 95:6756-6761; Bohlmann J., et al. (1999) Arch Biochem. Biophys. 368:232-243; Chen, X., et al. (1996) J. Nat. Prod. 59:944-951; Colby, S. M., et al. (1993). Biol. Chem. 268:23016-23024; Csekf, L., et al. (1998) Mol. Bio. Evol. 15:1491-1498; Davis, E. M., et al. (1998) Plant Physiol. 116:1192; Facchini, P. J., and Chappell, J. (1992) Proc. Natl. Acad. Sci. USA 89:11088-11092; Mau, C. J. D. and West, C. A. (1994) Proc. Natl. Acad. Sci. USA 91:8479-8501; Steele, C. L., et al. (1998) J. Biol. Chem. 273:2078-2089; Stofer Vogel, B., et al. (1996) J. Biol. Chem. 271:23262-23268; Sun, T. and Kamiya, Y. (1994) Plant Cell 6:1509-1518; Sun, T. P., et al. (1992) Plant Cell 4:119-128; Wildung, M. R. and Croteau, R. (1996) J. Biol. Chem. 271:9201-9204; Yamaguchi, S., et al. (1998) Plant Physiol. 116:1271-1278; and Yuba, A., et al. (1996) Arch. Biochem. Biophys. 332:280-287.

In addition to the terpene synthases in Table 1, additional exemplary terpene synthases include Bisobolene synthase, (−)-Pinene synthase, 6-Selinene synthase, (−)-Limonene synthase, Abeitadiene synthase, and Taxadiene synthase.

Examples of synthases include, but are not limited to, botryococcene synthase, limonene synthase, 1,8 cineole synthase, α-pinene synthase, camphene synthase, (+)-sabinene synthase, myrcene synthase, abietadiene synthase, taxadiene synthase, farnesyl pyrophosphate synthase, amorphadiene synthase, (E)-α-bisabolene synthase, diapophytoene synthase, or diapophytoene desaturase. Additional examples of enzymes useful in the disclosed embodiments are described in Table 2.

### Table 2

### Examples of Enzymes Involved in the Isoprenoid Pathway

TABLE 2
Examples of Enzymes Involved in the Isoprenoid Pathway

| Enzyme | Source | NCBI protein ID |
|---|---|---|
| Limonene | M. spicata | 2ONH_A |
| Cineole | S. officinalis | AAC26016 |
| Pinene | A. grandis | AAK83564 |
| Camphene | A. grandis | AAB70707 |
| Sabinene | S. officinalis | AAC26018 |
| Myrcene | A. grandis | AAB71084 |
| Abietadiene | A. grandis | Q38710 |
| Taxadiene | T. brevifolia | AAK83566 |
| FPP | G. gallus | P08836 |
| Amorphadiene | A. annua | AAF61439 |
| Bisabolene | A. grandis | O81086 |
| Diapophytoene | S. aureus | |
| Diapophytoene desaturase | S. aureus | |
| GPPS-LSU | M. spicata | AAF08793 |
| GPPS-SSU | M. spicata | AAF08792 |
| GPPS | A. thaliana | CAC16849 |
| GPPS | C. reinhardtii | EDP05515 |
| FPP | E. coli | NP_414955 |
| FPP | A. thaliana | NP_199588 |
| FPP | A. thaliana | NP_193452 |
| FPP | C. reinhardtii | EDP03194 |
| Limonene | L. angustifolia | ABB73044 |
| Monoterpene | S. lycopersicum | AAX69064 |
| Terpinolene | O. basilicum | AAV63792 |
| Myrcene | O. basilicum | AAV63791 |
| Zingiberene | O. basilicum | AAV63788 |
| Myrcene | Q. ilex | CAC41012 |
| Myrcene | P. abies | AAS47696 |
| Myrcene, ocimene | A. thaliana | NP_179998 |
| Myrcene, ocimene | A. thaliana | NP_567511 |
| Sesquiterpene | Z. mays; B73 | AAS88571 |
| Sesquiterpene | A. thaliana | NP_199276 |
| Sesquiterpene | A. thaliana | NP_193064 |
| Sesquiterpene | A. thaliana | NP_193066 |
| Curcumene | P. cablin | AAS86319 |
| Farnesene | M. domestica | AAX19772 |
| Farnesene | C. sativus | AAU05951 |
| Farnesene | C. junos | AAK54279 |
| Farnesene | P. abies | AAS47697 |
| Bisabolene | P. abies | AAS47689 |
| Sesquiterpene | A. thaliana | NP_197784 |
| Sesquiterpene | A. thaliana | NP_175313 |
| GPP Chimera | | |
| GPPS-LSW + SSU fusion | | |
| Geranylgeranyl reductase | A. thaliana | NP_177587 |
| Geranylgeranyl reductase | C. reinhardtii | EDP09986 |
| FPP A118W | G. gallus | |

The synthase may also be β-caryophyllene synthase, germacrene A synthase, 8-epicedrol synthase, valencene synthase, (−)-δ-cadinene synthase, germacrene C synthase, (E)-β-farnesene synthase, casbene synthase, vetispiradiene synthase, 5-epi-aristolochene synthase, aristolochene synthase, α-humulene, (E,E)-α-farnesene synthase, (−)-β-pinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, γ-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, or manool synthase.

### Nucleic Acids, Proteins, and Enzymes

The vectors and other nucleic acids disclosed herein can encode polypeptide(s) that promote the production of intermediates, products, precursors, and derivatives of the products (e.g., terpenes and terpenoids) described herein. For example, the vectors can encode polypeptide(s) that promote the production of intermediates, products, precursors, and derivatives in the isoprenoid pathway.

The enzymes utilized in practicing the present disclosure may be encoded by nucleotide sequences derived from any organism, including bacteria, plants, fungi and animals. In some instances, the enzymes are terpene synthases. As used herein, a “terpene synthase” is a naturally or non-naturally occurring enzyme which produces or increases production of terpene/terpenoids and/or their derivatives. Terpenes/terpenoids of the present disclosure can be monoterpenes, diterpenes, triterpenes, sesquiterpenes, or any other naturally or non-naturally occurring terpene. In some embodiments, the terpene is fusicoccadiene. In some instances, a terpene synthase of the present disclosure is fusicoccadiene synthase, producing fusicoccadiene. In other instances, a terpene synthase of the present disclosure catalyzes the conversion of IPP and/or DMAPP into a terpene/terpenoid of interest, such as fusicoccadiene. The enzymes may have one or more distinct catalytic activities, such as prenyltransferase activity arid/or terpene cyclase activity. In some embodiments, a host cell may be genetically modified so as to produce more than one exogenous or endogenous polypeptide (e.g., enzyme) which, in combination results in the production of a desired product (e.g., terpene/terpenoid). In some instances, the polypeptides may be naturally occurring polypeptides. In other instances, the polypeptides and/or the genes encoding them may be modified from their natural state, including, but not limited to functional truncations, genetic modifications, or synthetically synthesized polynucleotides. Polynucleotides encoding enzymes and other proteins useful in the present disclosure may be isolated and/or synthesized by any means known in the art, including, but not limited to cloning, sub-cloning, and PCR. Exemplary DNA manipulations are described in Sambrook et al., Molecular Cloning: A Laboratory Manual (Cold Spring Harbor Laboratory Press 1989) and Cohen et al., Meth. Enzymol. 297, 192-208, 1998.

An expression vector, including, but not limited to, regulatory elements and sequences encoding genes, may comprise nucleotide sequences that are codon biased for expression in the organism being transformed. Therefore, when synthesizing, for example, a gene for expression in a host cell, it may be desirable to design the gene such that its frequency of codon usage approaches the frequency of the preferred codon usage of the host cell. In some instances, a native (unmodified) gene may exhibit a complete or partial match to the codon bias of the intended target host cell. In such instances, little or no codon optimization need be performed. In some organisms, codon bias differs between the nuclear genome and organelle genomes, thus, codon optimization or biasing may be performed for the target genome (e.g., nuclear codon biased or chloroplast codon biased). The codons of the host organism may be, for example, A/T rich in the third nucleotide position. Often, A/T rich codon bias is used for algae. In some embodiments, at least 50% of the third nucleotide position of the codons are A or T. In other embodiments, at least 60%, at least 70%, at least 80%, at least 90%, at least 95%, or at least 99% of the third nucleotide position of the codons are A or T.

One or more codons of an encoding polynucleotide can be biased to reflect chloroplast and/or nuclear codon usage. Most amino acids are encoded by two or more different (degenerate) codons, and it is well recognized that various organisms utilize certain codons in preference to others. Such preferential codon usage, which also is utilized in chloroplasts, is referred to herein as “chloroplast codon usage”. The codon bias of Chlamydomonas reinhardtii has been reported. See U.S. Application 2004/0014174. Percent identity to the native sequence (in the organism from which the sequence was isolated) may be about 50%, about 60%, about 70%, about 80%, about 90%, about 95%, about 99% or higher.

The term “biased,” when used in reference to a codon, means that the sequence of a codon in a polynucleotide has been changed such that the codon is one that is used preferentially in the target which the bias is for, e.g., alga cells, or chloroplasts. A polynucleotide that is biased for chloroplast codon usage can be synthesized de novo, or can be genetically modified using routine recombinant DNA techniques, for example, by a site-directed mutagenesis method, to change one or more codons such that they are biased for chloroplast codon usage. Chloroplast codon bias can be variously skewed in different plants, including, for example, in alga chloroplasts as compared to tobacco. Generally, the chloroplast codon bias selected reflects chloroplast codon usage of the plant which is being transformed with the nucleic acids of the present disclosure. For example, where C. reinhardtii is the host, the chloroplast codon usage is biased to reflect alga chloroplast codon usage (about 74.6% AT bias in the third codon position).

The terms “hot” codon bias or “regular” codon bias are used broadly here to refer to different types of artificially introduced codon bias to a gene. “Regular” codon bias refers to a codon bias closely following the codon usage of the host organism into which the gene is introduced. Such regular codon bias can involve the alteration of one or more codons from the native sequence to a codon preferred in a host organism. In some instances, a host organism will have different codon usages in different genomes. For example, the chloroplast genome of C. reinhardtii has a different codon bias than the nuclear genome. Therefore, codon biasing typically will reflect the targeted genome within the host cell.

“Hot” codon bias is similar to regular codon bias in that one or more codons from a native sequence are changed to reflect codon usage in the host organism. For “hot” codon bias, the synthetic gene contains the codon most frequently used by the host genome to encode the desired amino acid at that position, unless use of that codon would introduce an undesired restriction enzyme recognition sequence at a given position. For instance, there are three codons that encode the amino acid isoleucine, ATC, ATT, and ATA. In the Chlamydomonas chloroplast genome, the codon ATT is used 77% of the time, ATC is used 12% of the time, and ATA is used 11% of the time. In a “hot” codon biased gene, the codon ATT will therefore be used at all positions where isoleucine is to be encoded, unless use of ATT would introduce an undesired restriction enzyme recognition site.

Nucleic Acid and Amino Acid Sequences Useful in the Disclosed Embodiments

SEQ ID NO:1 Phomopsis amygdali fusicoccadiene synthase (PaFS) nucleotide sequence

SEQ ID NO:2 PaFS protein sequence

SEQ ID NO:3 Strep-Tag amino acid sequence including TG linker

SEQ ID NO:4 “Regular” codon optimized PaFS nucleotide sequence without tag

SEQ ID NO:5 “Regular” codon optimized PaFS nucleotide sequence with C-terminal Strep Tag

SEQ ID NO:6 Amino acid sequence of PaFS with C-terminal Strep Tag

SEQ ID NO:7 “Hot” codon optimized PaFS nucleotide sequence without tag

SEQ ID NO:8 “Hot” codon optimized PaFS nucleotide sequence with C-terminal Strep Tag

SEQ ID NO:9 Phaesosphaeria nodorum ent-Kaurene synthase nucleotide sequence

SEQ ID NO:10 Ent-Kaurene synthase protein sequence

SEQ ID NO:11 “Hot” codon optimized ent-Kaurene synthase nucleic acid sequence, without tag

SEQ ID NO:12 N-terminal FLAG tag amino acid sequence

SEQ ID NO:13 “Hot” codon optimized ent-Kaurene synthase nucleic acid sequence with N-terminal FLAG tag

SEQ ID NO:14 Amino acid sequence of ent-Kaurene synthase with N-terminal FLAG tag

SEQ ID NO:15 Ricinus communis casbene synthase nucleotide sequence

SEQ ID NO:16 Casbene synthase protein sequence

SEQ ID NO: 17 “Hot” codon optimized casbene synthase nucleic acid sequence, without tag

SEQ ID NO:18 “Hot” codon optimized casbene synthase nucleic acid sequence, with C-terminal strep tag including TGIN linker

SEQ ID NO:19 Strep tag amino acid sequence including TGIN linker

SEQ ID NO:20 Casbene synthase protein sequence with strep-tag

SEQ ID NO:21 Casbene synthase/GGPP synthase fusion protein nucleotide sequence, without tag

SEQ ID NO:22 Translation of Casbene synthase/GGPP synthase fusion protein without tag

SEQ ID NO:23 CLIP-8×his tag protein sequence

SEQ ID NO:24 Casbene synthase/GGPP synthase fusion protein nucleotide sequence including CLIP-8×his tag

SEQ ID NO:25 Casbene synthase/GGPP synthase fusion protein sequence including CLIP-8×his tag

SEQ ID NO:26 Abies grandis Abietadiene synthase gene nucleotide sequence

SEQ ID NO:27 Abietadiene synthase protein sequence

SEQ ID NO:28 Codon optimized abietadiene synthase nucleotide sequence without tag

SEQ ID NO:29 TEV-FLAG tag amino acid sequence

SEQ ID NO:30 Codon optimized abietadiene synthase nucleotide sequence with C-terminal TEV-FLAG tag

SEQ ID NO:31 Abietadiene synthase nucleotide sequence with C-terminal TEV-FLAG tag protein sequence

SEQ ID NO:32 Taxus brevifolia taxadiene synthase gene nucleotide sequence

SEQ ID NO:33 Taxadiene synthase protein sequence

SEQ ID NO:34 Codon optimized taxadiene synthase nucleotide sequence without tag

SEQ ID NO:35 Codon optimized taxadiene synthase nucleotide sequence with C-terminal TEV-FLAG tag protein sequence

SEQ ID NO:36 Taxadiene synthase nucleotide sequence with C-terminal TEV-FLAG tag protein sequence

SEQ ID NO:37 Prenyltransferase domain of fusicoccadiene synthase nucleotide sequence

SEQ ID NO:38 Prenyltransferase domain of fusicoccadiene synthase protein sequence

SEQ ID NO:39 “Hot” codon optimized prenyltransferase domain of fusicoccadiene synthase nucleotide sequence without tag

SEQ ID NO:40 “Hot” codon optimized prenyltransferase domain of fusicoccadiene synthase nucleotide sequence with C-terminal Strep Tag

SEQ ID NO:41 Prenyltransferase domain of fusicoccadiene synthase with C-terminal Strep Tag protein sequence

SEQ ID NO:42 Primer 1 from Example 12

SEQ ID NO:43 Primer 2 from Example 12

SEQ ID NO:44 Native nucleotide sequence encoding a hypothetical protein EAS27885 from C. immitis

SEQ ID NO:45 Translation of C. immitis protein EAS27885

SEQ ID NO:46 Codon optimized nucleotide sequence for C. immitis EAS27885 without tag

SEQ ID NO:47 C. immitis hypothetical protein nucleotide sequence as expressed (IS-92) with C-terminal strep tag

SEQ ID NO:48 C. immitis hypothetical protein translation as expressed (IS-92) with C-terminal strep tag

SEQ ID NO:49 Nucleotide sequence Encoding a hypothetical protein EAA68264 from G. zeae

SEQ ID NO:50 Translation of gene encoding hypothetical protein EAA68264 from G. zeae

SEQ ID NO:51 Codon optimized gene encoding hypothetical protein EAA68264 from G. zeae without tag

SEQ ID NO:52 Codon optimized gene encoding hypothetical protein EAA68264 from G. zeae nucleotide sequence as expressed with c-terminal strep tag

SEQ ID NO:53 Translation of gene encoding hypothetical protein EAA68264 from G. zeae nucleotide sequence as expressed with c-terminal strep tag

SEQ ID NO:54 Nucleotide sequence from Aspergillus clavatus NRRL1 encoding hypothetical protein ACLA_{—}076850

SEQ ID NO:55 Translation of nucleotide sequence from Aspergillus clavatus NRRL1 encoding hypothetical protein ACLA_{—}076850

SEQ ID NO:56 Codon optimized nucleotide sequence for hypothetical protein ACLA_{—}076850 without tags

SEQ ID NO:57 Codon optimized nucleotide sequence for hypothetical protein ACLA_{—}076850 as expressed, with c-terminal strep-tag

SEQ ID NO:58 Translation of Codon optimized nucleotide sequence for hypothetical protein ACLA_{—}076850 as expressed, with c-terminal strep-tag

SEQ ID NO:59 Primer 1 from Example 13

SEQ ID NO:60 Primer 2 from Example 13

### Percent Sequence Identity

One example of an algorithm that is suitable for determining percent sequence identity or sequence similarity between nucleic acid or polypeptide sequences is the BLAST algorithm, which is described, e.g., in Altschul et al., J. Mol. Biol. 215:403-410 (1990). Software for performing BLAST analysis is publicly available through the National Center for Biotechnology Information. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (as described, for example, in Henikoff & Henikoff (1989) Proc. Natl. Acad. Sci. USA, 89:10915). In addition to calculating percent sequence identity, the BLAST algorithm also can perform a statistical analysis of the similarity between two sequences (for example, as described in Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, less than about 0.01, or less than about 0.001.

A polynucleotide or nucleic acid of the present disclosure can encode more than one gene. For example, the polynucleotide can encode for a first gene and a second gene, or a first gene, a second gene, and a third gene. Furthermore, any or all of the genes can be the same or different.

The polypeptides expressed in host cells of the present disclosure, including yeast, bacteria, or a microalga such as C. reinhardtii may be assembled to form functional polypeptides and protein complexes. As such, one embodiment of the disclosure provides a method to produce functional protein complexes, including, for example, dimers, trimers, and tetramers, wherein the subunits of the complexes can be the same or different (e.g., homodimers or heterodimers, respectively).

A polynucleotide or nucleic acid molecule as described herein can contain two or more sequences that are linked in a manner such that the product is not found in a cell in nature. The two or more nucleotide sequences can be operatively linked and, for example, can encode a fusion polypeptide, or can comprise an encoding nucleotide sequence and a regulatory element. A nucleic acid molecule also can be based on, but manipulated so as to be different from a naturally occurring polynucleotide, (e.g. biased for chloroplast codon usage or a restriction enzyme site can be inserted into the nucleic acid). A nucleic acid molecule may further contain a peptide tag (e.g., His-6 tag), which can facilitate identification of expression of the polypeptide in a cell. Additional tags include, for example: a FLAG epitope; a c-myc epitope; Strep-TAGII; biotin; and glutathione S-transferase. Such tags can be detected by any method known in the art (e.g., anti-tag antibodies or streptavidin). Such tags may also be used to isolate the operatively linked polypeptide(s), for example by affinity chromatography.

A polynucleotide or nucleic acid sequence comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (for example, as described in Jellinek et al., Biochemistry 34:11363-11372, 1995). Polynucleotides or nucleic acids useful for practicing the present disclosure may be isolated from any organism.

### Products

Examples of products contemplated herein include hydrocarbon products and hydrocarbon derivative products. A hydrocarbon product is one that consists of only hydrogen molecules and carbon molecules. A hydrocarbon derivative product is a hydrocarbon product with one or more heteroatoms, wherein the heteroatom is any atom that is not hydrogen or carbon. Examples of heteroatoms include, but are not limited to, nitrogen, oxygen, sulfur, and phosphorus. Some products can be hydrocarbon-rich, wherein, for example, at least 50%, at least 60%, at least 70%, at least 80%, at least 90%, or at least 95% of the product by weight is made up of carbon and hydrogen.

One exemplary group of hydrocarbon products are isoprenoids. Isoprenoids (including terpenoids) are derived from isoprene subunits, but are modified, for example, by the addition of heteroatoms such as oxygen, by carbon skeleton rearrangement, and by alkylation. Isoprenoids generally have a number of carbon atoms which is evenly divisible by five, but this is not a requirement as “irregular” terpenoids are known to one of skill in the art. Carotenoids, such as carotenes and xanthophylls, are examples of isoprenoids that are useful products. A steroid is an example of a terpenoid. Examples of isoprenoids include, but are not limited to, hemiterpenes (C5), monoterpenes (C 10), sesquiterpenes (C15), diterpenes (C20), triterpenes (C30), tetraterpenes (C40), polyterpenes (Cₙ, wherein “n” is equal to or greater than 45), and their derivatives. Other examples of isoprenoids include, but are not limited to, limonene, 1,8-cineole, α-pinene, camphene, (+)-sabinene, myrcene, abietadiene, taxadiene, farnesyl pyrophosphate, fusicoccadiene, amorphadiene, (E)-α-bisabolene, zingiberene, or diapophytoene, and their derivatives.

Useful products include, but are not limited to, terpenes and terpenoids as described above. An exemplary group of terpenes are diterpenes (C20). Diterpenes are hydrocarbons that can be modified (e.g. oxidized, methyl groups removed, or cyclized); the carbon skeleton of a diterpene can be rearranged, to form, for example, terpenoids, such as fusicoccadiene. Fusicoccadiene may also be formed, for example, directly from the isoprene precursors, without being bound by the availability of diterpene or GGDP. Genetic modification of organisms, such as algae, by the methods described herein, can lead to the production of fusicoccadiene, for example, and other types of terpenes, such as limonene, for example. Genetic modification can also lead to the production of modified terpenes, such as methyl squalene or hydroxylated and/or conjugated terpenes such as paclitaxel.

Other useful products can be, for example, a product comprising a hydrocarbon obtained from an organism expressing a diterpene synthase. Such exemplary products include ent-kaurene, casbene, and fusicocaccadiene, and may also include fuel additives.

The products produced by the present disclosure may be naturally, or non-naturally (e.g., as a result of transformation) produced by the host cell(s) and/or organism(s) transformed. For example, products not naturally produced by algae may include non-native terpenes/terpenoids such as fusicoccadiene. The host cell may be genetically modified, for example, by transformation of the cell with a sequence encoding a protein, wherein expression of the protein results in the secretion of a non-naturally produced product or products.

Examples of useful products include petrochemical products and their precursors and all other substances that may be useful in the petrochemical industry. Products include, for example, petroleum products, precursors of petroleum, as well as petrochemicals and precursors thereof. The fuel or fuel products may be used in a combustor such as a boiler, kiln, dryer or furnace. Other examples of combustors are internal combustion engines such as vehicle engines or generators, including gasoline engines, diesel engines, jet engines, and other types of engines. Products described herein may also be used to produce plastics, resins, fibers, elastomers, pharmaceuticals, neutraceuticals, lubricants, and gels, for example.

Isoprenoid precursors are generated by one of two pathways; the mevalonate pathway or the methylerythritol phosphate (MEP) pathway (FIG. 2 and FIG. 3). Both pathways generate dimethylallyl pyrophosphate (DMAPP) and isopentyl pyrophosphate (IPP), the common C5 precursor for isoprenoids. The DMAPP and IPP are condensed to form geranyl-diphosphosphate (GPP), or other precursors, such as farnesyl-diphosphate (FPP) or geranylgeranyl-diphosphate (GGPP), from which higher isoprenoids are formed.

Useful products can also include small alkanes (for example, 1 to approximately 4 carbons) such as methane, ethane, propane, or butane, which may be used for heating (such as in cooking) or making plastics. Products may also include molecules with a carbon backbone of approximately 5 to approximately 9 carbon atoms, such as naptha or ligroin, or their precursors. Other products may be about 5 to about 12 carbon atoms, or cycloalkanes used as gasoline or motor fuel. Molecules and aromatics of approximately 10 to approximately 18 carbons, such as kerosene, or its precursors, may also be useful as products. Other products include lubricating oil, heavy gas oil, or fuel oil, or their precursors, and can contain alkanes, cycloalkanes, or aromatics of approximately 12 to approximately 70 carbons. Products also include other residuals that can be derived from or found in crude oil, such as coke, asphalt, tar, and waxes, generally containing multiple rings with about 70 or more carbons, and their precursors.

The various products may be further refined to a final product for an end user by a number of processes. Refining can, for example, occur by fractional distillation. For example, a mixture of products, such as a mix of different hydrocarbons with various chain lengths may be separated into various components by fractional distillation.

Refining may also include any one or more of the following steps, cracking, unifying, or altering the product. Large products, such as large hydrocarbons (e.g. ≧C10), may be broken down into smaller fragments by cracking. Cracking may be performed by heat or high pressure, such as by steam, visbreaking, or coking. Products may also be refined by visbreaking, for example by thermally cracking large hydrocarbon molecules in the product by heating the product in a furnace. Refining may also include coking, wherein a heavy, almost pure carbon residue is produced. Cracking may also be performed by catalytic means to enhance the rate of the cracking reaction by using catalysts such as, but not limited to, zeolite, aluminum hydrosilicate, bauxite, or silica-alumina. Catalysis may be by fluid catalytic cracking, whereby a hot catalyst, such as zeolite, is used to catalyze cracking reactions. Catalysis may also be performed by hydrocracking, where lower temperatures are generally used in comparison to fluid catalytic cracking. Hydrocracking can occur in the presence of elevated partial pressure of hydrogen gas. Products may be refined by catalytic cracking to generate diesel, gasoline, and/or kerosene.

The products may also be refined by combining them in a unification step, for example by using catalysts, such as platinum or a platinum-rhenium mix. The unification process can produce hydrogen gas, a by-product, which may be used in cracking.

The products may also be refined by altering, rearranging, or restructuring hydrocarbons into smaller molecules. There are a number of chemical reactions that occur in catalytic reforming processes which are known to one of ordinary skill in the arts. Catalytic reforming can be performed in the presence of a catalyst and a high partial pressure of hydrogen. One common process is alkylation. For example, propylene and butylene are mixed with a catalyst such as hydrofluoric acid or sulfuric acid, and the resulting products are high octane hydrocarbons, which can be used to reduce knocking in gasoline blends.

The products may also be blended or combined into mixtures to obtain an end product. For example, the products may be blended to form gasoline of various grades, gasoline with or without additives, lubricating oils of various weights and grades, kerosene of various grades, jet fuel, diesel fuel, heating oil, and chemicals for making plastics and other polymers. Compositions of the products described herein may be combined or blended with fuel products produced by other means.

Some products produced from the host cells of the disclosure, especially after refining, will be identical to existing petrochemicals, i.e. contain the same chemical structure. For instance, crude oil contains the isoprenoid pristane, which is thought to be a breakdown product of phytol, which is a component of chlorophyll. Some of the products may not be the same as existing petrochemicals. However, although a molecule may not exist in conventional petrochemicals or refining, it may still be useful in these industries. For example, a hydrocarbon could be produced that is in the boiling point range of gasoline, and that could be used as gasoline or an additive, even though the hydrocarbon does not normally occur in gasoline.

### Vectors

The organisms/host cells herein can be transformed to modify the production and/or secretion of a product(s) with an expression vector, or a linearized portion thereof, for example, to increase production and/or secretion of a product(s). The product(s) can be naturally or not naturally produced by the organism.

An expression vector, or a linearized portion thereof, can comprise one or more polynucleotides that comprise nucleotide sequences that are exogenous or endogenous to the host organism.

In some instances, a sequence to be inserted into a host cell genome (e.g., a nuclear genome or chloroplast genome) is flanked by two sequences. These flanking sequences include those that have at least 50%, at least 60%, at least 70%, at least 80%, at least 90%, at least 95%, or 100% sequence identity to the sequence found in the host cell. The flanking homologous sequences enable recombination of the exogenous or endogenous sequence into the genome of the host organism through homologous recombination. In some instances, the flanking homologous sequences can be at least 100, at least 200, at least 300, at least 400, at least 500, at least 1000, or at least 1500 nucleotides in length.

Any of the vectors described herein can further comprise a regulatory control sequence. A regulatory control sequence may include, for example, promoter(s), operator(s), repressor(s), enhancer(s), transcription termination sequence(s), sequence(s) that regulate translation, or other regulatory control sequence(s) that are compatible with the host cell and control the expression of the nucleic acid molecules of the present disclosure. In some cases, a regulatory control sequence includes transcription control sequence(s) that are able to control, modulate, or effect the initiation, elongation, and/or termination of transcription. For example, a regulatory control sequence can increase the transcription and/or translation rate and/or efficiency of a gene or gene product in an organism, wherein expression of the gene or gene product is upregulated resulting (directly or indirectly) in the increased production, secretion, or both, of a product described herein. The regulatory control sequence may also result in increased of production, secretion, or both, of a product by increasing the stability of a gene or gene product.

A regulatory control sequence can be exogenous or endogenous in relationship to the host organism. A regulatory control sequence may encode one or more polypeptides that are enzymes that promote expression and production of a desired product. For example, an exogenous regulatory control sequence may be derived from another species of the same genus of the organism (e.g., another algal species).

Regulatory control sequences that can be used in the disclosed embodiments can effect inducible or constitutive expression of a desired sequence. For example, algal regulatory control sequences can be used; these sequences can be of nuclear, viral, extrachromosomal, mitochondrial, or chloroplastic origin.

Suitable regulatory control sequences include those naturally associated with the nucleotide sequence to be expressed (for example, an algal promoter operably linked with an algal-derived nucleotide sequence in nature). Suitable regulatory control sequences also include regulatory control sequences not naturally associated with the nucleic acid molecule to be expressed (for example, an algal promoter of one species operatively linked to a nucleotide sequence of another organism or algal species).

A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading phase. Linking is achieved by ligation at restriction enzyme sites. If suitable restriction sites are not available, then synthetic oligonucleotide adapters or linkers can be used as is known to those skilled in the art. Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

To determine whether a putative regulatory control sequence is suitable, the putative regulatory control sequence can be linked to a nucleic acid molecule encoding a protein that produces a detectable signal. The construct comprising the putative regulatory control sequence and nucleic acid may then be introduced into an alga or other organism by standard techniques, and expression of the protein monitored. For example, if the nucleic acid molecule encodes a dominant selectable marker, the alga or organism to be used is tested for the ability to grow in the presence of a compound for which the marker provides resistance.

In some cases, a regulatory control sequence is a promoter, such as a promoter adapted for expression of a nucleotide sequence in a non-vascular, photosynthetic organism. For example, the promoter may be an algal promoter, for example as described in U.S. Publ. Appl. No. 2006/0234368, now U.S. Pat. No. 7,449,568, issued Nov. 11, 2008, and U.S. Publ. Appl. No. 2004/0014174, and in Hallmann, Transgenic Plant J. 1:81-98 (2007). The promoter may be a chloroplast specific promoter or a nuclear specific promoter. The promoter may an EF1-α gene promoter or a D promoter. In some embodiments, the polypeptide, for example a synthase, is operably linked to an EF1-α. gene promoter. In other embodiments, a synthase is operably linked to a D promoter. Other exemplary promoters that can be used in the embodiments disclosed herein include, but are not limited to, the psbA, psbD, tufA, rbcL, HSP70A, and RBCS2 promoters.

A regulatory control sequence can be placed in a construct in a variety of locations, including for example, within coding and non-coding regions, 5′ untranslated regions (e.g., regions upstream from the coding region), or 3′ untranslated regions (e.g., regions downstream from the coding region). Thus, in some instances a regulatory control sequence can include one or more 3′ or 5′ untranslated regions, one or more introns, or one or more exons.

For example, the vector can comprise a 5′ regulatory region. In some embodiments, the 5′ regulatory comprises a promoter. The vector can also comprise a 3′ regulatory region. The promoter can be a constitutive promoter or an inducible promoter. Examples of inducible promoters include, for example, a light inducible promoter, a nitrate inducible promoter, or a heat responsive promoter.

For example, in some embodiments, a regulatory control sequence can comprise a Cyclotella cryptica acetyl-CoA carboxylase 5′ untranslated regulatory control sequence or a Cyclotella cryptica acetyl-CoA carboxylase 3′-untranslated regulatory control sequence (for example, as described in U.S. Pat. No. 5,661,017).

A regulatory control sequence may also encode chimeric or fusion polypeptides, such as the protein AB or SAA, that promote expression of an endogenous or exogenous nucleotide sequence or protein. Other regulatory control sequences can include intron sequences that may promote translation of an endogenous or exogenous sequence.

The regulatory control sequences used in any of the vectors described herein may be inducible. Inducible regulatory control sequences, such as promoters, can be inducible by light, for example. Regulatory control sequences may also be autoregulatable. Examples of autoregulatable regulatory control sequences include those that are autoregulated by, for example, endogenous ATP levels or by the product produced by the organism. In some instances, the regulatory control sequences may be inducible by an exogenous agent. Other inducible elements are well known in the art and may be adapted for use in the present disclosure.

Various combinations of the regulatory control sequences described herein may be embodied by the present disclosure and combined with other features of the present disclosure. In some cases, an expression vector comprises one or more regulatory control sequences operatively linked to a nucleotide sequence encoding a polypeptide. Such sequences may, for example, upregulate secretion. production, or both, of a product described herein. In some cases, an expression vector comprises one or more regulatory control sequences operatively linked to a nucleotide sequence encoding a polypeptide that effects, for example, upregulates secretion, production, or both, of a product.

In some instances, such vectors include promoters. Promoters useful in the present disclosure may come from any source (e.g., viral, bacterial, fungal, protist, or animal). The promoters contemplated for use herein can be, for example, specific to photosynthetic organisms, prokaryotic or eukaryotic non-vascular photosynthetic organisms, vascular photosynthetic organisms (e.g., flowering plants), yeast, or non-photosynthetic bacteria. The promoter can be, for example, a promoter for expression in a chloroplast and/or other plastid organelle. Alternatively, the promoter can be a promoter for expression in a bacterial host including, for example, a cyanobacteria. In one example, the promoter is chloroplast based. Examples of promoters contemplated for use in the present disclosure include those disclosed in U.S. Application No.: 2004/0014174. The promoter can be a constitutive promoter or an inducible promoter. A promoter typically includes necessary nucleic acid sequences near the start site of transcription, (e.g., a TATA element).

A “constitutive” promoter is a promoter that is active under most environmental and developmental conditions. An “inducible” promoter is a promoter that is active under environmental or developmental regulation. Examples of inducible promoters/regulatory elements include, for example, a nitrate-inducible promoter (for example, as described in Bock et al, Plant Mol. Biol. 17:9 (1991)), or a light-inducible promoter, (for example, as described in Feinbaum et al, Mol Gen. Genet. 226:449 (1991); and Lam and Chua, Science 248:471 (1990)), or a heat responsive promoter (for example, as described in Muller et al., Gene 111: 165-73 (1992)).

To select integration sites and/or determine codon usage, the genome of C. reinhardtii can be consulted. The entire chloroplast genome of C. reinhardtii is available to the public on the world wide web, at the URL “http://www.chlamy.org/chloro/default.html”, which is incorporated herein by reference. The chloroplast genome is also described in GenBank Ace. No.: AF396929, and in Maul, J. E., et al., Plant Cell 14 (11), 2659-2679 (2002). Generally, a portion of the nucleotide sequence of the chloroplast genomic DNA is selected as an integration site, such that it is not a portion of a gene, a regulatory sequence or a coding sequence, especially where integration of exogenous DNA would produce a deleterious effect with respect to the chloroplast and/or host cell (e.g., replication of the chloroplast genome). In this respect, the website containing the C. reinhardtii chloroplast genome, the GenBank Acc. No.: AF396929, and Maul, J. E., et al., Plant Cell 14 (11), 2659-2679 (2002), all provide maps showing the coding and non-coding regions of the chloroplast genome, thus facilitating selection of a sequence useful for constructing a vector of the present disclosure. For example, the chloroplast vector, p322, is a clone extending from the Eco (Eco RI) site at about position 143.1 kb to the Xho (Xho I) site at about position 148.5 kb of the C. reinhardtii chloroplast genome (http.://www.chlamy.org/chloro/default.html).

A vector utilized in the practice of the disclosure also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, or sequences that encode a selectable marker. As such, the vector can contain, for example, one or more cloning sites such as a multiple cloning site, which can, but need not, be positioned such that an exogenous or endogenous polynucleotide can be inserted into the vector and operatively linked to a desired element.

The vector can also contain a prokaryote origin of replication (ori), for example, an E. coli ori or a cosmid ori, thus allowing maintenance of the vector into a prokaryote host cell, as well as in a plant chloroplast, as desired. In some instances, the vectors of the present disclosure will contain elements such as an S. cerevisiae origin of replication. Such features, combined with appropriate selectable markers, allows for the vector to be “shuttled” between the target host cell and a bacterial and/or yeast cell, for example. The ability to transfer a shuttle vector of the disclosure into a secondary host may allow for the more convenient manipulation of the features of the vector. For example, a reaction mixture comprising a vector comprising a polynucleotide of interest can be transformed into a prokaryote host cell such as E. coli, amplified, and collected using routine methods, and examined to identify vectors containing an insert, peptide, or construct of interest. If desired, the vector can be further manipulated, for example, by performing site-directed mutagenesis on the polynucleotide of interest, then again amplifying and selecting for vectors that have the mutated polynucleotide of interest. The shuttle vector can then be introduced into plant cell chloroplasts, for example, wherein the polypeptide of interest can be expressed and, if desired, isolated according to methods known to one of skill in the art.

A vector can also contain additional elements such as a regulatory element. A regulatory element, as the term is used herein, broadly refers to a nucleotide sequence that regulates the transcription or translation of a polynucleotide, or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, and an IRES. A regulatory element can be a cell compartmentalization signal, for example, a sequence that targets a polypeptide to the cytosol, nucleus, chloroplast membrane, or cell membrane. In some aspects of the present disclosure, a cell compartmentalization signal (e.g., a chloroplast targeting sequence) may be ligated to a gene and/or transcript, such that translation of the gene occurs in the chloroplast. In other aspects, a cell compartmentalization signal may be ligated to a gene such that, following translation of the gene, the protein is transported to the chloroplast. Such signals are well known in the art and have been widely reported (for example, as described in U.S. Pat. No. 5,776,689; Quinn et al., J. Biol. Chem. 1999; 274(20): 14444-54; and von Heijne et al., Eur. J. Biochem. 1989; 180(3): 535-45).

A vector, or a linearized portion thereof, may include a nucleotide sequence encoding a reporter polypeptide or other selectable marker. The term “reporter” or “selectable marker” refers to a polynucleotide (or encoded polypeptide) that confers a detectable phenotype. A reporter may encode a detectable polypeptide, for example, a green fluorescent protein or an enzyme such as luciferase, which, when contacted with an appropriate agent (a particular wavelength of light or luciferin, respectively) generates a signal that can be detected by the eye or by using appropriate instrumentation (for example, as described in Giacomin, Plant Sci. 116:59-72, 1996; Scikantha, J. Bacteriol. 178:121, 1996; Gerdes, FEBS Lett. 389:44-47, 1996; and Jefferson, EMBO J. 6:3901-3907, 1997, fl-glucuronidase). A selectable marker can be, for example, a molecule that, when present or expressed in a cell, provides a selective advantage (or disadvantage) to the cell containing the marker, for example, the ability to grow in the presence of an agent that otherwise would kill the cell.

A selectable marker can provide a means to obtain prokaryotic cells, plant cells, or both, that express the marker and, therefore, can be useful as a component of a vector of the disclosure (for example, as described in Bock, R. (2001) Journal of Molecular Biology 312(3) 425-438). One class of selectable markers are native or modified genes which restore a biological or physiological function to a host cell (e.g., restores photosynthetic capability or restores a metabolic pathway). Other examples of selectable markers include, but are not limited to, those that confer antimetabolite resistance, for example, dihydrofolate reductase, which confers resistance to methotrexate (for example, as described in Reiss, Plant Physiol. (Life Sci. Adv.) 13:143-149, 1994); neomycin phosphotransferase, which confers resistance to the aminoglycosides neomycin, kanamycin, and paromycin (for example, as described in Herrera-Estrella, EMBO J. 2:987-995, 1983), hygro, which confers resistance to hygromycin (for example, as described in Marsh, Gene 32:481-485, 1984), trpB, which allows cells to utilize indole in place of tryptophan; hisD, which allows cells to utilize histinol in place of histidine (for example, as described in Hartman, Proc. Natl. Acad. Sci., USA 85:8047, 1988); mannose-6-phosphate isomerase which allows cells to utilize mannose (for example, as described in WO 94/20627); ornithine decarboxylase, which confers resistance to the ornithine decarboxylase inhibitor, 2-(difluoromethyl)-DL-ornithine (DFMO; for example, as described in McConlogue, 1987, In: Current Communications in Molecular Biology, Cold Spring Harbor Laboratory ed.); and deaminase from Aspergillus terreus, which confers resistance to Blasticidin S (for example, as described in Tamura, Biosci. Biotechnol. Biochem. 59:2336-2338, 1995). Additional selectable markers include those that confer herbicide resistance, for example, a phosphinothricin acetyltransferase gene, which confers resistance to phosphinothricin (for example, as described in White et al., Nucl. Acids Res. 18:1062, 1990; and Spencer et al., Theor. Appl. Genet. 79:625-631, 1990), a mutant EPSPV-synthase, which confers glyphosate resistance (for example, as described in Hinchee et al., BioTechnology 91:915-922, 1998), a mutant acetolactate synthase, which confers imidazolione or sulfonylurea resistance (for example, as described in Lee et al., EMBO J. 7:1241-1248, 1988), a mutant psbA, which confers resistance to atrazine (for example, as described in Smeda et al., Plant Physiol. 103:911-917, 1993), a mutant protoporphyrinogen oxidase (for example, as described in U.S. Pat. No. 5,767,373), or other markers conferring resistance to a herbicide such as glufosinate. Selectable markers include, for example, polynucleotides that confer dihydrofolate reductase (DHFR), neomycin, and tetracycline resistance for eukaryotic cells; ampicillin resistance for prokaryotes such as E. coli; and bleomycin, gentamycin, glyphosate, hygromycin, kanamycin, methotrexate, phleomycin, phosphinotricin, spectinomycin, streptomycin, sulfonamide, and sulfonylurea resistance in plants (for example, as described in Maliga et al., Methods in Plant Molecular Biology, Cold Spring Harbor Laboratory Press, 1995, page 39).

Reporter genes have been successfully used in chloroplasts of higher plants, and high levels of recombinant protein expression have been reported. In addition, reporter genes have been used in the chloroplast of C. reinhardtii. Reporter genes greatly enhance the ability to monitor gene expression in a number of biological organisms. For example, in the chloroplasts of higher plants, β-glucuronidase (uidA, for example, as described in Staub and Maliga, EMBO J. 12:601-606, 1993), neomycin phosphotransferase (nptII, for example, as described in Carrer et al., Mol. Gen. Genet. 241:49-56, 1993), adenosyl-3-adenyltransferase (aadA, for example, as described in Svab and Maliga, Proc. Natl. Acad. Sci., USA 90:913-917, 1993), and Aequorea victoria GFP (for example, as described in Sidorov et al., Plant J. 19:209-216, 1999), have been used as reporter genes (as described in Heifetz, Biochemie 82:655-666, 2000). Each of these genes has attributes that make them useful reporters of chloroplast gene expression, such as ease of analysis, sensitivity, or the ability to examine expression in situ. Proteins, such as Bacillus thuringiensis Cry toxins, have been expressed in the chloroplasts of higher plants, conferring resistance to insect herbivores (for example, as described in Kota et al., Proc. Natl. Acad. Sci., USA 96:1840-1845, 1999). Human somatotropin (for example, as described in Staub et al., Nat. Biotechnol. 18:333-338, 2000), a potential biopharmaceutical, has also been expressed. In addition, several reporter genes have been expressed in the chloroplast of the eukaryotic green alga, C. reinhardtii, including aadA (for example, as described in Goldschmidt-Clermont, Nucl. Acids Res. 19:4083-4089 1991; and Zerges and Rochaix, Mol. Cell Biol. 14:5268-5277, 1994), uidA (for example, as described in Sakamoto et al., Proc. Natl. Acad. Sci., USA 90:477-501, 19933; and Ishikura et al., J. Biosci. Bioeng. 87:307-314 1999), Renilla luciferase (for example, as described in Minko et al., Mol. Gen. Genet. 262:421-425, 1999), and the amino glycoside phosphotransferase from Acinetobacter baumnanii, aphA6 (for example, as described in Bateman and Purton, Mol. Gen. Genet. 263:404-410, 2000).

A gene encoding a protein of interest may be fused to a molecular marker or tag. In some instances, the tag may be an epitope tag or a tag polypeptide. For example, epitope tags can comprise a sufficient number of amino acid residues to provide an epitope against which an antibody cart be made, yet is short enough such that it does not interfere with the activity of the polypeptide to which it is fused. A tag may be unique so that an antibody raised to the tag does not substantially cross-react with other epitopes (e.g., a FLAG tag). Other appropriate tags that may be used, for example, are affinity tags. Affinity tags are appended to proteins so that they can be purified from their crude biological source using an affinity technique. Examples of such tags include, but are not limited to, chitin binding protein (CBP), maltose binding protein (MBP), glutathione-s-transferase (GST), a Strep-TagII tag, and metal affinity tags (e.g., pol(His). Positioning of tag(s) at the C- and/or N-terminal may be determined based on, for example, protein function. One of skill in the art will recognize that selection of an appropriate tag and its location in relationship to the protein of interest will be based on multiple factors, including for example, the intended use of the protein and the target protein itself.

One approach to construction of a genetically manipulated organism (e.g., algal strain) involves transformation with a nucleic acid which encodes a gene of interest, for example, a gene encoding fusicoccadiene synthase. In some embodiments, a transformation may introduce nucleic acids into any plastid of the host alga cell (e.g., chloroplast). In other embodiments, a transforming vector may be extrachromosomal (e.g., does not integrate into a genome). The organism transformed can be an alga. In still other embodiments, bacteria or yeast are transformed. Transformed cells are typically plated on selective media following the introduction of exogenous nucleic acids. This method may also comprise several steps for screening. Initially, a screen of primary transformants is typically conducted to determine which clones have proper insertion of the exogenous nucleic acids. Clones which show the proper integration arid/or vector capture may be propagated and re-screened to ensure genetic stability. Such methodology ensures that the transformants contain the genes of interest. In many instances, such screening is performed by polymerase chain reaction (PCR); however, any other appropriate technique known in the art may be utilized.

Many different methods of PCR are known in the art (e.g., nested PCR or real time PCR). For any given screen, one of skill in the art will recognize that PCR components may be varied to achieve optimal screening results. For example, magnesium concentration may need to be adjusted upwards when PCR is performed on disrupted alga cells to which EDTA (which chelates magnesium) is added to chelate toxic metals. In such instances, magnesium concentration may need to be adjusted upward, or downward (compared to the standard concentration in commercially available PCR kits) by about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 mM. Thus, after adjusting, the final magnesium concentration in a PCR reaction may be, for example about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5 mM or higher. Several examples provided below utilize PCR, however, one of skill in the art will recognize that other PCR techniques may be substituted for the particular protocols described. Following screening for clones with proper integration of exogenous nucleic acids, clones are typically screened for the presence of the encoded protein. Protein expression screening can be performed by Western blot analysis and/or enzyme activity assays.

A polynucleotide or recombinant nucleic acid molecule of the disclosure can be introduced into host cells, including bacteria, yeast, and algae, chloroplasts or nuclei using any method known in the art. A polynucleotide can be introduced into a cell by a variety of methods, which are well known in the art and selected, in part, based on the particular host cell. For example, when a bacteria, is used as a host cell, the expression vector can be introduced into the host cell by any conventional method known to one of skill in the art, such as a calcium chloride or electroporation, as described, for example, in Molecular Cloning (J. Sambrook et al., Cold spring H-arbor, 1989). When yeast is used as a host cell, the expression vector can be introduced into the host cell using a lithium or spheroplast transformation technique, for example. In addition, a polynucleotide can be introduced into a plant cell using various techniques. Such techniques include, but are not limited to: a direct gene transfer technique such as electroporation; microprojectile mediated (biolistic) transformation using a particle gun; a “glass bead method”; pollen-mediated transformation; liposome-mediated transformation; transformation using wounded or enzyme-degraded immature embryos; or transformation using wounded or enzyme-degraded embryogenic callus (for example, as described in Potrykus, Ann. Rev. Plant. Physiol. Plant Mol. Biol. 42:205-225, 1991).

The term “exogenous” is used herein in a comparative sense to indicate that a nucleotide sequence (or polypeptide) being referred to is from a source other than a reference source, is linked to a second nucleotide sequence (or polypeptide) with which it is not normally associated, or is modified such that it is in a form that is not normally associated with a reference material.

Plastid transformation is a method for introducing a polynucleotide into a plant cell chloroplast (for example, as described in U.S. Pat. Nos. 5,451,513, 5,545,817, and 5,545,818; WO 95/16783; and McBride et al., Proc. Natl. Acad. Sci., USA 91:7301-7305, 1994). In some embodiments, chloroplast transformation involves introducing a desired nucleotide sequence flanked by regions of chloroplast DNA, allowing for homologous recombination of the nucleotide sequence into the target chloroplast genome.

One of skill in the art will recognize that host cells, transformed with a vector as described above, include transformation with a circular or a linearized vector, or a linearized portion of a vector. In some instances, one to 1.5 kb flanking nucleotide sequences of chloroplast genomic DNA may be used. Smaller regions of flanking sequences can be used. One of skill in the art would be able to determine the size of the flanking region that should be used without undue experimentation. Using this method, point mutations in the chloroplast 16S rRNA and rps12 genes, which confer resistance to spectinomycin and streptomycin, can be utilized as selectable markers for transformation (for example, as described in Svab et al., Proc. Natl. Acad. Sci., USA 87:8526-8530, 1990), and can result in stable homoplasmic transformants, at a frequency of approximately one per 100 bombardments of target leaves.

Microprojectile mediated transformation also can be used to introduce a polynucleotide into a plant cell chloroplast (for example, as described in Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a plant tissue using a device such as the BIOLISTIC PD-1000 particle gun (BioRad; Hercules Calif.). Methods for the transformation using biolistic methods are well known in the art (see, e.g.; Christou, Trend in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (for example, as described in Duan et al., Nature Biotech. 14:494-498, 1996; and Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, and the glass bead agitation method.

Transformation frequency may be increased by replacement of recessive rRNA or r-protein antibiotic resistance genes with a dominant selectable marker, including, but not limited to the bacterial aadA gene (for example, as described in Svab and Maliga, Proc. Natl. Acad. Sci., USA 90:913-917, 1993). For example, approximately 15 to 20 cell division cycles following transformation may be required to reach a homoplastidic state. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein.

A method of the disclosure can be performed by introducing a recombinant nucleic acid molecule into a chloroplast or into the nucleus of a cell, wherein the recombinant nucleic acid molecule includes a first polynucleotide, which encodes at least one polypeptide (i.e., 1, 2, 3, 4, or more). In some embodiments, a polypeptide is operatively linked to a second, third, fourth, fifth, sixth, seventh, eighth, ninth, tenth and/or subsequent polypeptide. For example, several enzymes in a hydrocarbon production pathway may be linked, either directly or indirectly, such that products produced by one enzyme in the pathway, once produced, are in close proximity to the next enzyme in the pathway.

For transformation of chloroplasts, one aspect of the present disclosure is the utilization of a recombinant nucleic acid construct which contains both a selectable marker and one or more genes of interest. In one instance, transformation of chloroplasts is performed by co-transformation of chloroplasts with two constructs: one containing a selectable marker and a second containing the gene(s) of interest. The time required to grow some transformed organisms may be lengthy. The transformants are then screened both for the presence of the selectable marker and for the presence of the gene(s) of interest. Typically, secondary screening for the gene(s) of interest is performed by Southern blot.

In chloroplasts, regulation of gene expression generally occurs after transcription, and often during translation initiation. This regulation is dependent upon the chloroplast translational apparatus, as well as nuclear-encoded regulatory factors (for example, as described in Barkan and Goldschmidt-Clermont, Biochemie 82:559-572, 2000; and Zerges, Biochemie 82:583-601, 2000). The chloroplast translational apparatus generally resembles that of bacteria; chloroplasts contain 70S ribosomes; have mRNAs that lack 5′ caps and generally do not contain 3′ poly-adenylated tails (for example, as described in Harris et al., Microbiol. Rev. 58:700-754, 1994); and translation is inhibited in chloroplasts and in bacteria by selective agents such as chloramphenicol.

Some methods of the present disclosure take advantage of proper positioning of a ribosome binding sequence (RBS) with respect to a coding sequence, for example, a polynucleotide of interest. It has previously been noted that such placement of an RBS results in robust translation in plants (for example, as described in U.S. Application 2004/0014174, incorporated herein by reference). An advantage of expressing polypeptides in chloroplasts is that the polypeptides do not proceed through cellular compartments typically traversed by polypeptides expressed from a nuclear gene and, therefore, are not subject to certain post-translational modifications such as glycosylation. As such, the polypeptides and protein complexes produced by some methods of the disclosure can be expected to be produced without such post-translational modification.

The terms “polynucleotide”, “nucleic acid”, “nucleotide sequence”, or “nucleic acid molecule”, or similar terms known to one of skill in the art, are used broadly herein to mean a sequence of two or more deoxyribonucleotides or ribonucleotides that are linked together by a phosphodiester bond. As such, these terms are used interchangeably throughout the specification. These ter-is include, but are not limited to, RNA and DNA, a gene or a portion thereof, a cDNA, or a synthetic polydeoxyribonucleic acid sequence, and can be single stranded or double stranded, as well as a DNA/RNA hybrid. Furthermore, these terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR).

The nucleotides comprising a polynucleotide can be naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and are commercially available, as are polynucleotides containing such nucleotide analogs (for example, as described in Lin et al., Nucl. Acids Res. 22:5220-5234, 1994; Jellinek et al., Biochemistry 34:11363-11372, 1995; and Pagratis et al., Nature Biotechnol. 15:68-73, 1997). A phosphodiester bond can link the nucleotides of a polynucleotide of the present disclosure; however other bonds, for example, including a thiodiester bond, a phosphorothioate bond, a peptide-like bond, and any other bond known in the art may be utilized to produce synthetic polynucleotides (for example, as described in Tam et al., Nucl. Acids Res. 22:977-986, 1994; and Ecker and Crooke, BioTechnology 13:351360, 1995).

Any of the products described herein can be prepared by transforming an organism to cause the production and/or secretion by such organism of the product. An organism is considered to be a photosynthetic organism even if a transformation event destroys or diminishes the photosynthetic capability of the transformed organism (e.g., exogenous nucleic acid is inserted into a gene encoding a protein required for photosynthesis).

Any of the expression vectors described herein may be adapted for expression of a desired nucleic acid in a chloroplast or nucleus of a host organism. A number of chloroplast promoters from higher plants have been identified, for example, as described in Kung and Lin, Nucleic Acids Res. 13: 7543-7549 (1985). A chloroplast can be transformed by an expression vector comprising a nucleic acid sequence that encodes for a protein. In one embodiment the protein may be targeted to the chloroplast by a chloroplast targeting sequence. For example, targeting an expression vector or the gene product(s) encoded by an expression vector to the chloroplast may further enhance the effects provided by the regulatory control sequences described herein, and may effect the expression of a protein or peptide that allows for or improves the accumulation of a fuel molecule,

The concept of chloroplast targeting described herein may be combined with other features of the present disclosure. For example, a nucleotide sequence encoding a terpene synthase (e.g., fusicoccadiene synthase) may be operably linked to a nucleotide sequence encoding a chloroplast targeting sequence and the “linked” sequence then cloned into an expression vector. A host cell is then transformed with the expression vector and may produce more of the synthase as compared to a host cell transformed with an expression vector encoding terpene synthase but not a chloroplast targeting sequence. The increased terpene synthase expression may also result in more of the terpene (e.g., fusicoccadiene) being produced.

In yet another example, an expression vector comprising a nucleotide sequence encoding an enzyme that produces a product (e.g. fuel product, fragrance product, or insecticide product), not naturally produced by the organism, by using precursors that are naturally produced by the organism as substrates, is targeted to the chloroplast. By targeting the enzyme to the chloroplast, production of the product may be increased in comparison to a host cell, wherein the enzyme is expressed, but not targeted to the chloroplast. Without being bound by theory, this may be due to increased precursors being produced in the chloroplast and thus, more products may be produced by the enzyme encoded by the introduced nucleotide sequence.

### Modification of Enzymes

Various methods may be used to generate a variant polypeptide, for example, a variant terpene synthase. In some embodiments, variant polypeptide enzymes are generated by look-through mutagenesis, walk-through mutagenesis, gene shuffling, directed evolution, or sexual PCR. These methods allow for the generation of variant polypeptides containing random sequence(s), variant polypeptides made using predetermined modifications of particular residues, variant polypeptides that utilize evolutionary traits from different genes, and variant polypeptides that combine characteristics/functions of different parent genes.

The method of walk-through mutagenesis comprises introducing a predetermined amino acid into each and every position in a predefined region (or several different regions) of the amino acid sequence of a parent polypeptide. Walk-through mutagenesis is further described in greater detail in U.S. Pat. No. 5,798,208, which is hereby incorporated by reference in its entirety.

Look-through mutagenesis comprises introducing a predetermined amino acid into a selected set of positions, or a position, within a defined region (or several different regions) of the amino acid sequence of a parent polypeptide. Look-through mutagenesis is further described in greater detail in US Patent Publication No.: 2008/0214406, which is hereby incorporated by reference in its entirety.

Gene shuffling is a method for recursive in vitro or in vivo homologous recombination of pools of nucleic acid fragments or polynucleotides. Mixtures of related nucleic acid sequences or polynucleotides are randomly fragmented, and reassembled to yield a library or mixed population of recombinant nucleic acid molecules or polynucleotides. The equivalents of some standard genetic matings may also be performed by “gene shuffling” in vitro. For example, a “molecular backcross” can be performed by repeated mixing of the mutant's nucleic acid with the wild-type nucleic acid while selecting for the mutations of interest. In one example of in vivo shuffling, the mixed population of the specific nucleic acid sequence is introduced into bacterial or eukaryotic cells under conditions such that at least two different nucleic acid sequences are present in each host cell.

Variant polypeptides of the disclosure having altered properties can also be produced using “Sexual PCR.” In such an approach, amplified or cloned polynucleotides possessing a desired characteristic (for example, encoding a polypeptide with a region of higher specificity to a substrate) are selected (via screening of a library of polynucleotides, for example) and pooled.

Variant polypeptides of the disclosure having altered properties can also be produced using “Sequence Saturation Mutagenesis”. In such an approach, every nucleotide in a selected range of nucleotides is randomized using an early termination/extension protocol, described in Wong et al. (2004) Nucleic Acids Research, 32(3):e26.

Other techniques known to one skilled in the art can be used to generate variant polypeptides that can be used in the disclosed embodiments.

### Host, Organism

Examples of organisms that can be transformed using the compositions and methods herein include prokaryotic or eukaryotic organisms. In some instances, the organism is photosynthetic and can be vascular or non-vascular. Organisms useful herein can be of unicellular or multicellular organism.

A host organism is an organism comprising a host cell. In some embodiments, the host organism is photosynthetic. A photosynthetic organism is one that naturally photosynthesizes (has a plastid) or that is genetically engineered or otherwise modified to be photosynthetic. In some instances, a photosynthetic organism may be transformed with a construct of the disclosure which renders all or part of the photosynthetic apparatus inoperable. In some instances a host organism is non-vascular and photosynthetic. In some embodiments, the host organism is prokaryotic. Examples of some prokaryotic organisms of the present disclosure include, but are not limited to, cyanobacteria (e.g., Synechococcus, Synechocystis, Athrospira, Gleocapsa, Oscillatoria, and Pseudoanabaena) and E. coli. The host organism can be unicellular or multicellular. In some embodiments, the host organism is eukaryotic, for example, algae (e.g., microalgae, macroalgae, green algae, red algae, or brown algae) or fungi (e.g., yeast such as S. cerevisiae, Sz. pombe, and Candida spp.). In one embodiment, the green algae is Chlorphycean. In some embodiments, the host cell is a microalga. Examples of organisms contemplated herein include, but are not limited to, rhodophyta, chlorophyta, heterokontophyta, tribophyta, glaucophyta, chlorarachniophytes, euglenoids, haptophyta, cryptomonads, dinoflagellata, and phytoplankton.

As used herein, the term “non-vascular photosynthetic organism,” refers to any macroscopic or microscopic organism, including, but not limited to, algae, protists (such as euglena), cyanobacteria and other photosynthetic bacteria, which does not have a vascular system such as that found in higher plants. Examples of non-vascular photosynthetic organisms include bryophytes, such as marchantiophytes or anthocerotophytes. In some instances, the organism is a cyanobacteria, or algae (e.g., macroalgae or microalgae). The algae can be unicellular or multicellular algae. The algae can be a species of Chlamydomonas, Scenedesmus, Chlorella, or Nannochloropsis, for example. Examples of microalga include, but are not limited to, Chlamydomonas reinhardtii, D. salina, H. pluvalis, S. dimorphus, Chlorella vulgaris, N. salina, N. oculata, D. viridis, and D. tertiolecta. For example, the microalgae Chlamydomonas reinhardtii may be transformed with a vector, or a linearized portion thereof, encoding a fusicoccadiene synthase. In another embodiment, the alga is C. reinhardtii 137c.

In another instances, the organism can be a photosynthetic bacterium. A photosynthetic bacterium can be, for example, a member of the genus Synechocystis, Synechococcus, or Athrospira,

Also described herein are methods for utilizing non-photosynthetic bacteria as hosts to produce, for example, terpenoids, in some instances, the terpenoid is, for example, fusicoccadiene. Non-photosynthetic bacteria can be useful for producing terpenoids as non-metabolized products. In addition, various E. Coli strains, such as BL 21 or Bacillus spp. can be used in the present disclosure.

Genetic modifications of yeast host cells can be accomplished by complementation, transformation, homologous recombination, or other methods known to one of skill in the art. Genetic modification of bacterial cells can be accomplished, for example, by transient or stable transformation, or by modification of the bacterial genome. Techniques for transforming bacteria are well known to one of skill in the art.

As described above, methods and compositions of the present disclosure can also be performed using prokaryotic or eukaryotic organisms, for example, microorganisms. In addition to photosynthetic bacteria, non-photosynthetic bacteria including, but not limited to, Escherichia coli and Bacillus spp. can be utilized as host organisms for the embodiments disclosed herein. Additionally, fungi, in particular yeasts including, but not limited to Saccharomyces cerevisiae, Schizosaccharomcyes pombe, and Candida spp. can be utilized as host organisms for the embodiments disclosed herein.

The methods and compositions of the disclosure can be practiced using any plant having chloroplasts, including, for example, microalga and macroalgae. Examples of such plants are marine algae and seaweed, as well as plants that grow in soil.

Methods and compositions of the disclosure can generate a plant (e.g., alga) containing chloroplasts or a nucleus that is genetically modified to contain a stably integrated polynucleotide (for example, as described in Hager and Bock, Appl. Microbial. Biotechnol. 54:302-310, 2000). Accordingly, the present disclosure further provides a transgenic (transplastomic) plant, which comprises one or more chloroplasts and/or a nucleus comprising a polynucleotide encoding one or more endogenous or exogenous polypeptides (such as a terpene/terpenoid synthase), including a polypeptide or polypeptides that can specifically associate to form a functional protein complex, for example, a fusicoccadiene synthase.

In a one embodiment, the photosynthetic organism is a plant. The term “plant” is used broadly herein to refer to a eukaryotic organism containing plastids, particularly chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Exemplary useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, are seeds, fruits, cuttings, seedlings, tubers, rootstocks, and the like.

In other embodiments the photosynthetic organism is a vascular plant. Non-limiting examples of such plants include various monocots and dicots, including high oil seed plants such as high oil seed Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oilnut trees such as olive (Olea europaea), sesame, and peanut (Arachis hypogaea), as well as Arabidopsis, tobacco, wheat, barley, oats, amaranth, potato, rice, tomato, and legumes (e.g., peas, beans, lentils, alfalfa, etc.).

One of skill in the art will recognize that the organisms listed herein are merely representative of the possible host organisms that can be used in any of the disclosed embodiments, and are not limiting examples.

Some of the host organisms which may be used to practice the present disclosure are halophilic (e.g., Dunaliella salina, D. viridis, or D. tertiolecta). For example, D. salina can grow in ocean water, salt lakes (salinity from about 30 to about 300 parts per thousand), and high salinity media (e.g., artificial seawater medium, seawater nutrient agar, brackish water medium, or seawater medium, for example). In some embodiments of the disclosure, a host cell comprising a vector of the present disclosure can be grown in a liquid environment which is about 0.1, about 0.2, about 0.3, about 0.4, about 0.5, about 0.6. about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 31., about 3.2, about 3.3, about 3.4, about 3.5, about 3.6, about 3.7, about 3.8, about 3,9, about 4.0, about 4.1, about 4.2, about 4.3 molar, or higher concentrations of sodium chloride. One of skill in the art will recognize that other salts (sodium salts, calcium salts, sulfate salts, or potassium salts, for example) may also be present in the liquid environment.

Where a halophilic organism is utilized for the present disclosure, it may be transformed with any of the vectors described herein. For example, D. salina may be transformed with a vector which is capable of insertion into the chloroplast genome and which contains nucleic acids which encode a terpene producing enzyme (e.g., fusicoccadiene synthase). Transformed halophilic organisms may then be grown in high-saline environments (e.g., salt lakes, salt ponds, or high-saline media, for example) to produce the product(s) of interest. Isolation of the product(s) may involve removing a transformed organism from a high-saline environment prior to extracting the product(s) from the organism. In instances where the product is secreted into the surrounding environment, it may be necessary to desalinate the liquid environment prior to any further processing of the product.

Host cells can be grown under conditions which result in the production of a desired product, such as a terpene or terpenoid (e.g., fusicoccadiene). One of skill in the art will recognize that different growth conditions will be required, depending on the host cell. For example, where an alga (e.g., C. reinhardtii) is the host organism, growth in a liquid environment containing sufficient nitrogen, phosphorous and other essential elements may be required. In another example, where a non-photosynthetic bacterium such as E. coli is a host cell, growth on solid or liquid media may be appropriate to induce production of the desired product. In some instances, the growth environment is an aqueous environment.

A host organism may be grown under conditions which permit photosynthesis, however, this is not a requirement (e.g., a host organism may be grown in the absence of light). In some instances, the host organism may be genetically modified in such a way that its photosynthetic capability is diminished and/or destroyed. In growth conditions where a host organism is not capable of photosynthesis (e.g., because of the absence of light and/or genetic modification), typically, the organism will be provided the necessary nutrients to support growth in the absence of photosynthesis. For example, a culture medium in (or on) which an organism is grown, may be supplemented with any required nutrient, including an organic carbon source, nitrogen source, phosphorous source, vitamins, metals, lipids, nucleic acids, micronutrients, and/or any organism-specific requirement. Organic carbon sources include any source of carbon which the host organism is able to metabolize including, but not limited to, acetate, simple carbohydrates (e.g., glucose, sucrose, or lactose), complex carbohydrates (e.g., starch or glycogen), proteins, and lipids. One of skill in the art will recognize that not all organisms will be able to sufficiently metabolize a particular nutrient and that nutrient mixtures may need to be modified from one organism to another in order to provide the appropriate nutrient mix.

A host organism transformed to produce a protein described herein, for example, a synthase, can be grown on land, e.g., ponds, aqueducts, landfills, or in closed or partially closed bioreactor systems. Organisms, such as algae, can be grown directly in water, for example, in oceans, seas, lakes, rivers, or reservoirs. In embodiments where algae are mass-cultured, the algae can be grown in high density photobioreactors. Methods of mass-culturing algae are known in the art. For example, algae can be grown in high density photobioreactors (see, for example, Lee et al, Biotech. Bioengineering 44:1161-1167, 1994) and other bioreactors (such as those for sewage and waste water treatments) (for example, as described in Sawayama et al, Appl. Micro. Biotech., 41:729-731, 1994). Additionally, algae may be mass-cultured to remove heavy metals (for example, as described in Wilkinson, Biotech. Letters, 11:861-864, 1989), hydrogen (for example, as described in U.S. Patent Application Publication No. 20030162273), and pharmaceutical compounds.

In some cases, host organism(s) are grown near ethanol production plants or other facilities or regions (e.g., cities or highways, for example) generating CO₂. As such, the methods discussed herein include business methods for selling carbon credits to ethanol plants or other facilities or regions generating CO₂ while making fuels by growing one or more of the modified organisms described herein near the ethanol production plant.

In some embodiments, the pH of the media in which the host organism is grown may be controlled. The pH may be controlled using the addition of various acids. The acids used to control pH may include CO₂, nitric acid, phosphoric acid, or other acids. The pH of the media may be controlled to remain within the range of about pH 7.5 to about 8, about 8 to about 8.5, about 8.5 to about 9, about 9 to about 9,5, about 9.5 to about 10, about 10 to about 10.5, about 10.5 to about 11, or about 11 to about 11.5.

As discussed above, the organisms may be grown in outdoor open water, such as ponds, the ocean, the sea, rivers, waterbeds, marsh water, shallow pools, lakes, or reservoirs, for example. When grown in water, the organisms can be contained in a halo-like object comprising lego-like particles. The halo object encircles the algae and allows it to retain nutrients from the water beneath, while keeping it in open sunlight.

In some instances, organisms can be grown in containers wherein each container comprises 1 or 2 or a plurality of organisms. The containers can be configured to float on water. For example, a container can be filled by a combination of air and water to make the container and the host organism(s) in it buoyant. A host organism that is adapted to grow in fresh water can thus be grown in salt water (i.e., the ocean) and vice versa. This mechanism allows for the automatic death of the organism if there is any damage to the container.

In some instances a plurality of containers can be contained within a halo-like structure as described above. For example, up to 100, up to 1,000, up to 10,000, up to 100,000, up to 1,000,000, or more containers can be arranged in a meter-square of a halo-like structure.

In some embodiments, the product (e.g. fuel product) is collected by harvesting the organism. The product may then be extracted from the organism. In some instances, the product may be produced without killing the organisms. Producing and/or expressing the product may not render the organism unviabie. In other instances, the product may be secreted into a growing environment.

The product-containing biomass can be harvested from its growth environment (e.g. lake, pond, photobioreactor, or partially closed bioreactor system, for example) using any suitable method. Non-limiting examples of harvesting techniques are centrifugation or flocculation. Once harvested, the product-containing biomass can be subjected to a drying process. Alternately, an extraction step may be performed on wet biomass. The product-containing biomass can be dried using any suitable method. Non-limiting examples of drying methods include sunlight, rotary dryers, flash dryers, vacuum dryers, ovens, freeze dryers, hot air dryers, microwave dryers and superheated steam dryers. After the drying process the product-containing biomass can be referred to as a dry or semi-dry biomass.

In some embodiments, the production of the product (e.g. fuel product, fragrance product, or insecticide product) is inducible. The product may be induced to be expressed and/or produced, for example, by exposure to light. In yet other embodiments, the production of the product is autoregulatable. The product may form a feedback loop, wherein when the product (e.g. fuel product, fragrance product, or insecticide product) reaches a certain level, expression or secretion of the product may be inhibited. In other embodiments, the level of a metabolite of the organism may inhibit expression or secretion of the product. For example, endogenous ATP produced by the organism as a result of increased energy production to express or produce the product, may form a feedback loop to inhibit expression of the product. In yet another embodiment, production of the product may be inducible, for example, by an exogenous agent. For example, an expression vector for effecting production of a product in the host organism may comprise an inducible regulatory control sequence that is activated or inactivated by an exogenous agent.

The following examples are intended to provide illustrations of the application of the present disclosure. The following examples are not intended to completely define or otherwise limit the scope of the disclosure.

### EXAMPLES

### Example 1

### Synthesis of Codon Biased Genes Encoding Fusicoccadiene Synthase

A nucleic acid (SEQ ID NO: 1) encoding Phomopsis amygdali fusicoccadiene synthase (SEQ ID NO: 2) (gene product BAF45924.1, termed “PaFS”) was synthesized by DNA 2.0 in two different codon biases; one codon optimized by DNA 2.0 according to their usual algorithm using the C. reinhardtii chloroplast optimization (“regular” bias; IS87; SEQ ID NO: 4), the other utilized the most frequent C. reinhardtii codon at each amino acid position except where a change was necessary to eliminate undesired restriction sites (“hot” codon bias; IS88; SEQ ID NO: 7). In both cases, DNA encoding the amino acid sequence of SEQ ID NO: 3 was fused directly to the C-terminus to add an AgeI restriction enzyme site to the gene, and to add the Strep-TagII sequence for affinity purification and detection. The resulting amino acid sequence is shown in SEQ ID NO: 6.

### Example 2

### Production of Fusicoccadiene In Vitro by Recombinant Fusicoccadiene Synthase

The codon biased PaFS with a Strep tag II described in Example 1 above, was introduced into E. coli BL-21 cells. In this instance, the nucleic acid sequence encoding fusicoccadiene synthase with a Strep tag II (SEQ ID NO: 8) was ligated into the plasmid pST7, a customized vector using a T7 promoter and terminator and containing NdeI and XbaI sites for addition of the synthetic fusicoccadiene gene. The resulting plasmid was transformed into E. coli BL-21 (DE3) pLysS cells (Novagen). All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

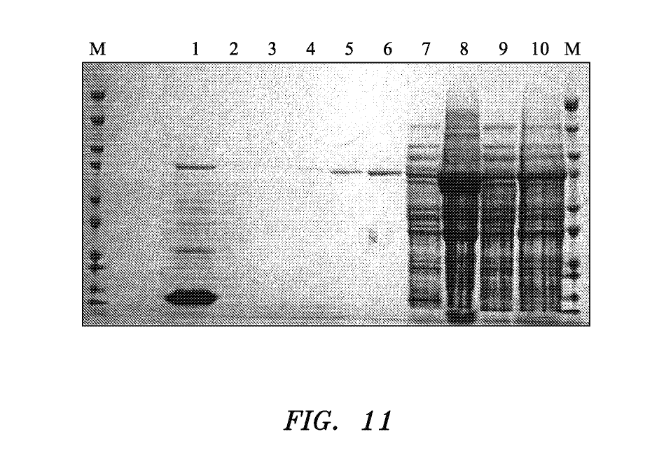

Expression of IS-88 (“hot” codon optimized fusicoccadiene synthase; encoded by the nucleic acid sequence of SEQ ID NO: 8) in a bacterial host under control of the T7 promoter was induced with IPTG. The bacteria were lysed by microfluidization, clarified by centrifugation, and the supernatant was applied to Streptactin resin (Qiagen, Inc.) used according to manufacturers instructions. The resin was washed and then the bound protein was eluted with desthiobiotin, as instructed. The samples were run on an SDS-PAGE gel, stained with coomassie brilliant blue, and imaged. Results are shown in FIG. 11 (Lanes: M=molecular weight marker; 1=:Resin; 2=Elution 5; 3=Elution 4; 4=Elution 3; 5=Elution 2; 6=Elution 1; 7=Flow through; 8=Pellet; 9=Clarified; 10=Crude Lysate). A fraction of the crude cell lysate was extracted with heptane and analyzed by Gas Chromatography using a Mass Selective Detector (GC/MSD). The results showed accumulation of fusicoccadiene in cells. This was identified by an essential oils mass spectrum library match and by comparison with the GC/MSD spectrum presented in Toyomasu T. et al. (2007), PNAS 104(9):3084-3088.

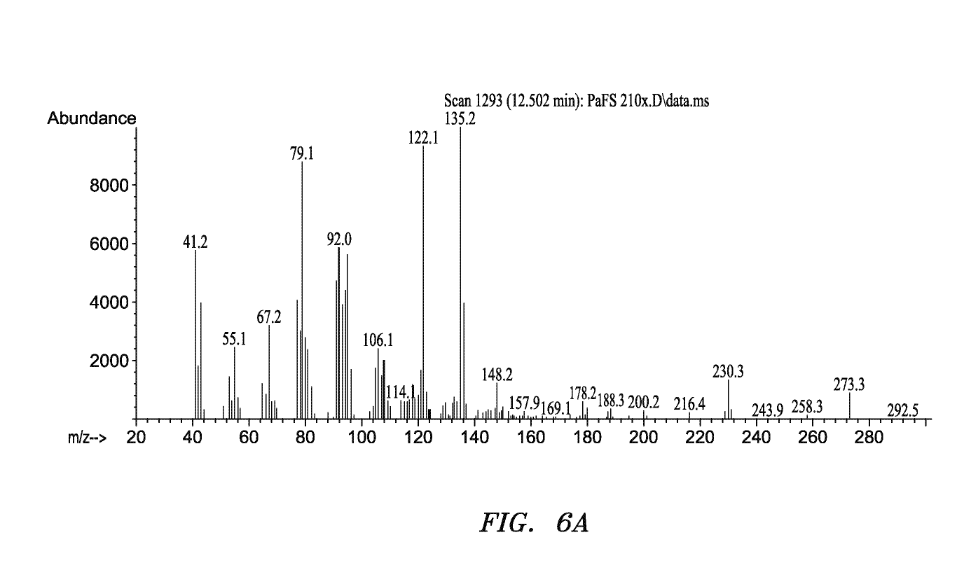

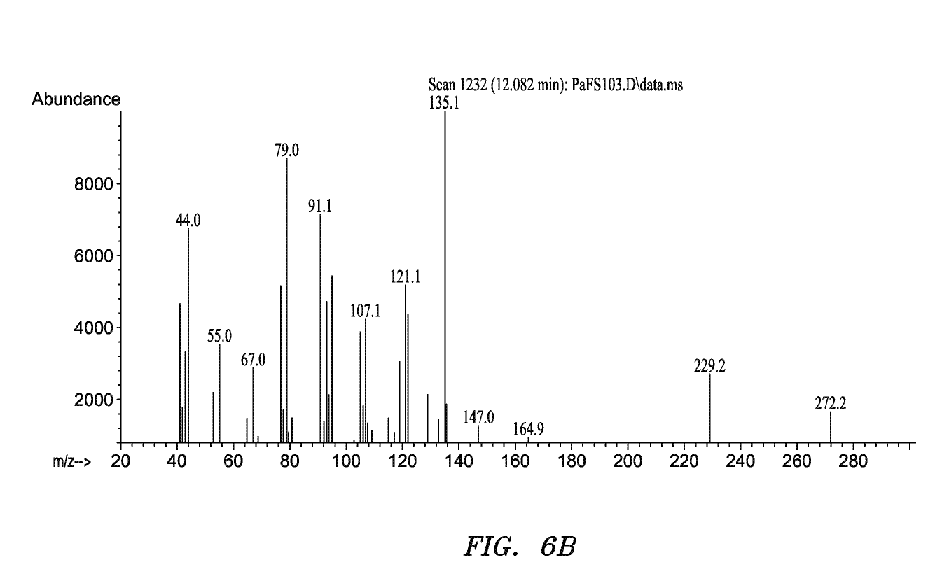

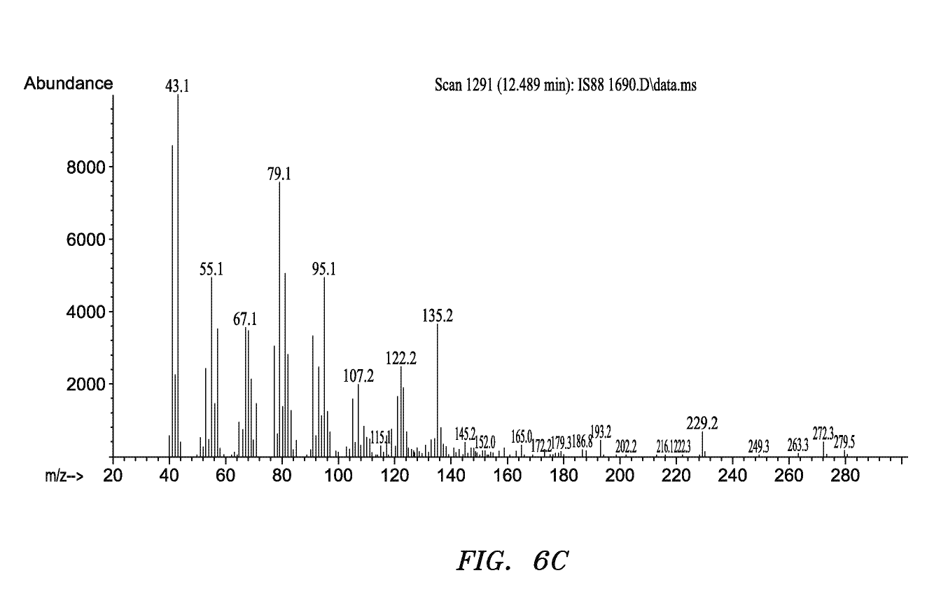

The purified protein was also assayed for activity. The enzyme was incubated in an assay mixture containing IPP and 1-¹³C-DMAPP (DMAPP with one carbon uniformly labeled with ¹³C). The products of the reaction were extracted with heptane and analyzed by GC/MSD. During the interval between the first experiment, this, and following experiments, the GC column was changed, resulting in a small change in retention time as the column length was increased. The result is shown in FIG. 6A, demonstrating the mass spectrum of the product (both the m/Z 272 molecular ion and the m/Z 229 fragment) was shifted by +1 amu (peak eluted at 12.50 min).

### Example 3

### Biosynthesis of Fusicocca-2 10(14)-Diene in E. coli In Vivo

The codon biased PaFS (SEQ ID NO: 8) with a Strep tag II described in Example 1 was cloned into a bacterial expression vector behind the T7 promoter as described in Example 2. The bacterial gene construct was transformed into BL21 (DE3) pLysS cells (Novagen), grown, and induced with IPTG at 17° C. for 36 hours. After induction, the cells were collected by centrifugation, lysed, and extracted with chloroform. The chloroform extract was dried in a rotary evaporator, and the residue was dissolved in heptane. The sample was analyzed by GC/MSD (FIG. 6B) and found to contain fusicoccadiene (peak eluted at 12.08 minutes).

### Example 4

### Algal Expression of Fusicoccadiene Synthase

The “hot” codon biased PaFS with a Strep tag II (encoded by the nucleic acid sequence of SEQ ID NO: 8) described in Example I was cloned into two algal expression vectors: 1) Chlamydomonas expression vector pSE-3HB-Kan-tD2; a vector containing a Kanamycin resistance gene driven by the Chlamydomonas atpA promoter, fusicoccadiene synthase driven by the tD2 promoter (i.e., a truncated Chlamydomonas D2 promoter), and flanked by homologous regions to drive integration into the Chlamydomonas chloroplast genome 3HB site; 2) Chlamydomonas expression vector pSE-D1-Kan; a vector containing a Kanamycin resistance gene driven by the Chlamydomonas atpA promoter, fusicoccadiene synthase driven by the D1 promoter, and flanked by homologous regions to drive integration into the Chlamydomonas chloroplast genome D1 site resulting in replacement of the native D1 gene.

The algal expression vector pSE-3HB-Kan-tD2 containing SEQ ID NO:8 was introduced into the chloroplast of the algal host strains (strain backgrounds 1690 and 137c, both mating type positive) using biolistic gold followed by growth on TAP plates with kanamycin selection (50 μg/ml). Colonies were screened for homoplasmicity and the presence of the fusicoccadiene synthase gene by PCR, Cultures (2 ml) of gene positive, homoplasmic algae were collected by centrifugation, resuspended in 250 μl of methanol. 500 μl of saturated NaCl in water and 500 μl of petroleum ether were added to the resuspended cultures. The solution was vortexed for three minutes, then centrifuged at 14,000×g for five minutes at room temperature to separate the organic and aqueous layers. The organic layer (100 μl) was transferred to a vial insert in a standard 2 ml sample vial and analyzed using GC/MSD, on the same column as in Example 2. The mass spectrum at 12,49 minutes for one sample (IS-88, PaFS with the “hot” codon bias under the D2 promoter, in the 1690 algal background) was obtained. The diagnostic ions at m/Z=272, 229, 135, 122, 107, 95, and 79 are present in this spectrum, demonstrating the presence of fusicocca-2,10 (14)-diene (FIG. 6C).

### Example 5

### Codon Optimization of PaFS in Algal Host Cells with Different Genetic Background

Two codon optimizations of PaFS for algal expression were tested. As described above, “regular” codon bias was applied to a nucleic acid encoding PaFS by DNA 2.0 software to generate sequence IS-87 (SEQ ID NO: 5). Sequence IS-88 (SEQ ID NO: 8) was generated by replacing all codons of PaFS with the codons most frequently used in the C. reinhardtii chloroplast genome except where such a replacement would introduce an undesirable feature such as a restriction enzyme site.

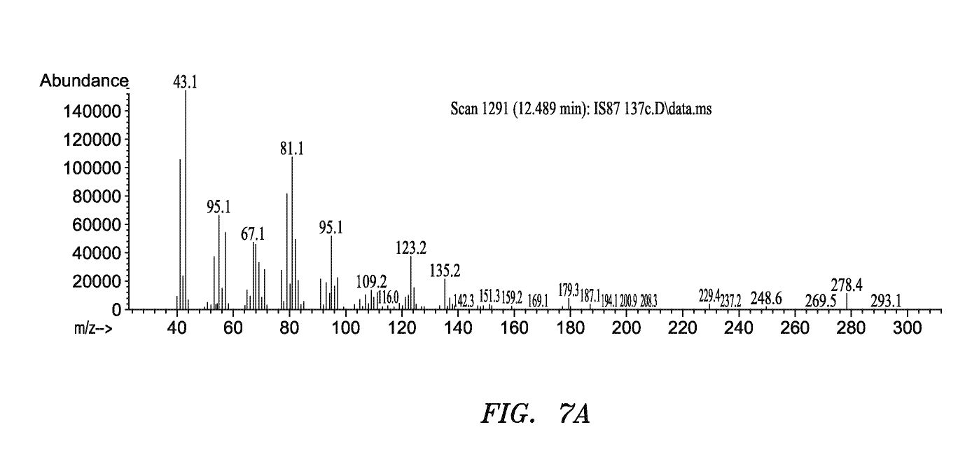

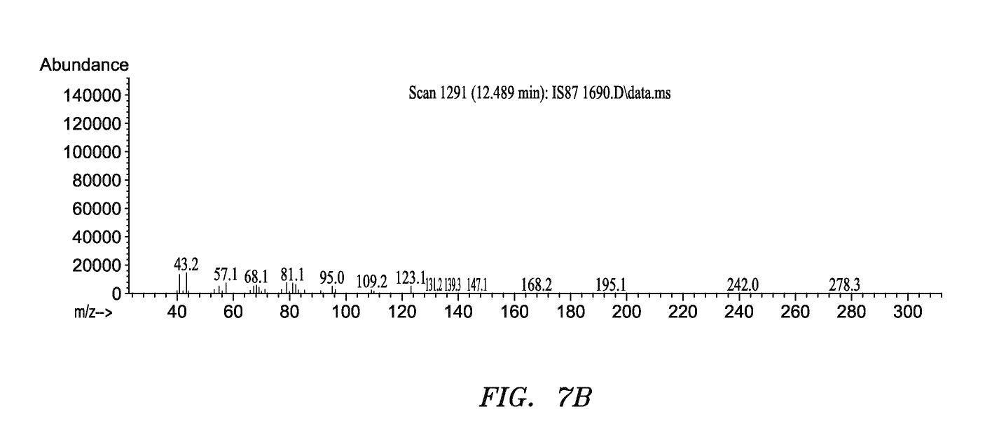

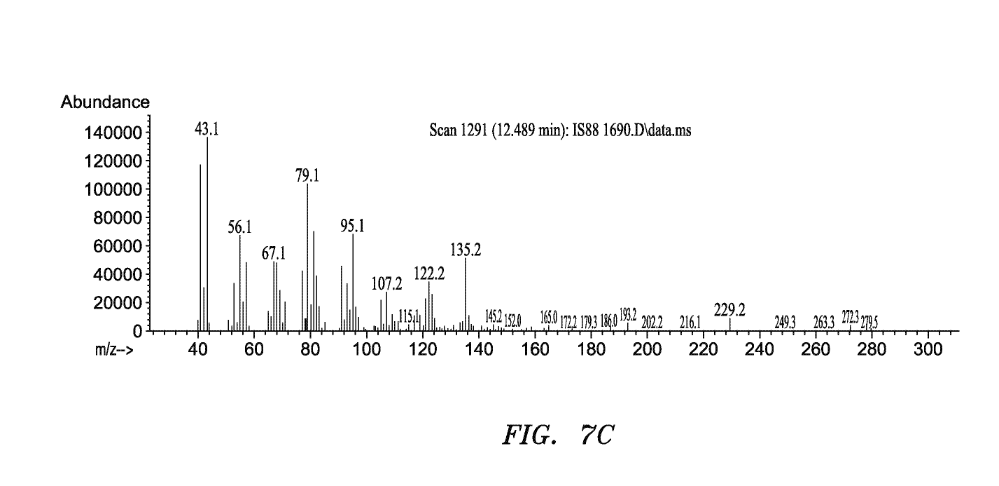

Three algal samples were extracted as described in Example 4 (replacing the petroleum ether with heptane) and analyzed by GC/MSD. FIG. 7A shows the mass spectrum for an algal extract from cells containing PaFS with regular codon bias in the C. reinhardtii 137c genetic background at 12.49 minutes post-injection. FIG. 7B shows the mass spectrum of an algal extract from wild type C. reinhardtii 1690 cells that lack the PaFS gene according to PCR screening (gene negative). Finally FIG. 7C shows the mass spectrum for an algal extract from cells containing the PaFS “hot” codon bias gene in C. reinhardtii 1690 from Example 4. The ions for fusicoccadiene are clearly present in FIG. 7A and FIG. 7C at m/z=229, 135, 123, and 95, and are absent in FIG. 7B. Of the differently optimized PaFS versions, the “Hot” codon optimized clone (SEQ ID NO:8) produced a much stronger fusicoccadiene signal than the “Regular” codon optimized clone (SEQ ID NO: 5).

Thin layer chromatography was performed to compare differently optimized PaFS versions (FIG. 8). In FIG. 8, lane one is fusicoccadiene produced in vivo by E. coli as described in Example 3. Lanes 2, 3, and 4 show the heptane extracts of Chlamydomonas cell cultures expressing genes IS-87 (regular codon bias fusicoccadiene synthase; encoded by the nucleic acid sequence of SEQ ID NO: 5), IS-88 (“hot” codon bias fusicoccadiene synthase; encoded by the nucleic acid sequence of SEQ ID NO: 8), or IS-89 (the nucleic acid sequence encoding the prenyltransferase domain of fusicoccadiene synthase) (SEQ ID NO: 40), 2 μl samples were spotted onto a silica gel TLC plate, developed with heptane, and stained with the general dye p-anisaldehyde. The spot near the top of the plate shows the purified fusicoccadiene.

### Example 6

### Production of Fusicoccadiene in Synechocystis sp. Strain PCC6803

The nucleic acid encoding the “hot” codon bias of PaFS (IS-88; SEQ ID NO: 8) was cloned into the cyanobacterium Synechocystis, downstream of the truncated IrtA promoter from PCC 6803, with the 3′-UTR of the gene encoding the S-layer protein from L. brevis as the terminator sequence. The truncated lrtA has previously been demonstrated to constitutively drive protein expression in PCC 6803. The regions of homology utilized for integration into the chromosome were from the 1 kb regions surrounding the psbY gene, a disposable subunit of the Synechocystis photosystem. The vector contains a kanamycin marker for antibiotic selection at a concentration of 5 ug/mL.

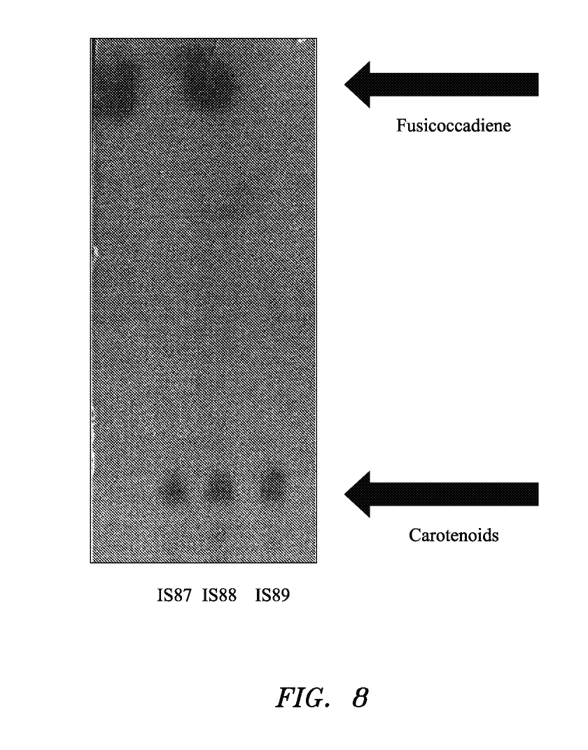

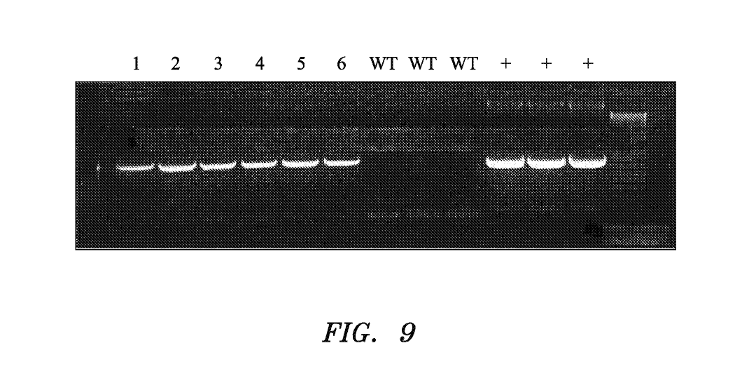

This DNA was introduced by natural transformation into Synechocystis sp strain PCC 6803 as follows. Liquid cultures of cells in log phase were concentrated to 10 million cells/mL and washed once with an excess volume of 10 mM NaCl. After removal of the salt solution, the cells were resuspended in an equal volume of nitrate-containing medium and treated with plasmid DNA at a concentration of 1 ug/mL. The cells and DNA were incubated at room temperature with shaking and 5% CO2 overnight while shaded from light. The following day, the cell suspension was plated onto a nitrate-containing agar plate in the presence of 5 ug/mL kanamycin. The plates were exposed to low light levels in the presence of CO_, for 3 days, and then shifted to high light conditions for 48 hrs to facilitate clearing. Upon appearance of colonies, clones were isolated, patched to another 5 ug/mL kanamycin plate, and incubated at room temperature with 5% CO₂ for an additional 5 days. Patches that grew colonies were subjected to colony PCR screening with primers specific to the “hot” codon bias of the fusicoccadiene synthase gene (termed PAFS103). Six gene-positive clones were identified (FIG. 9).

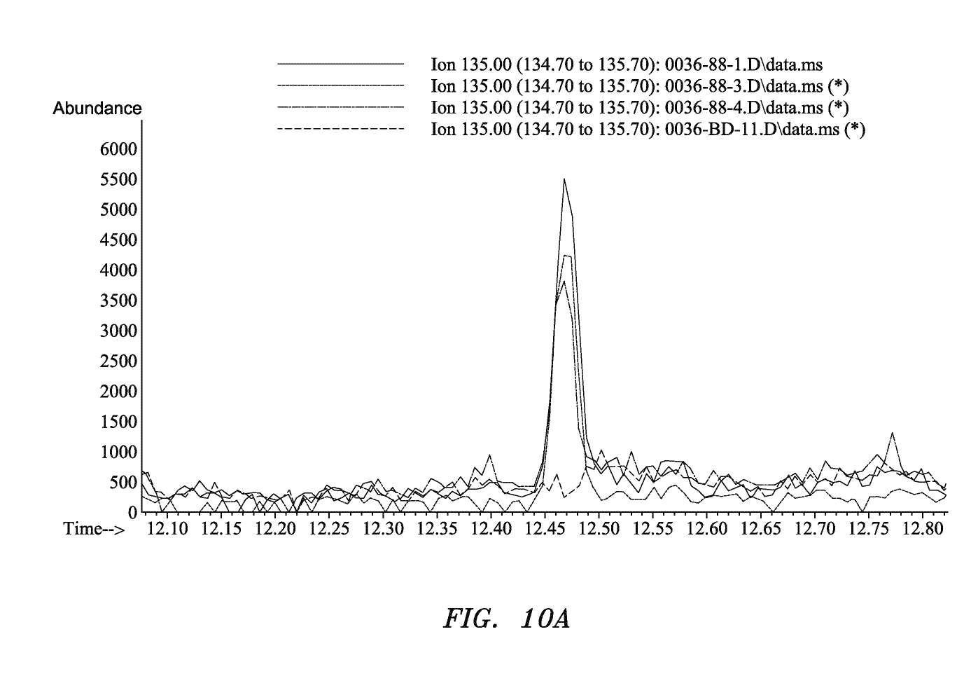

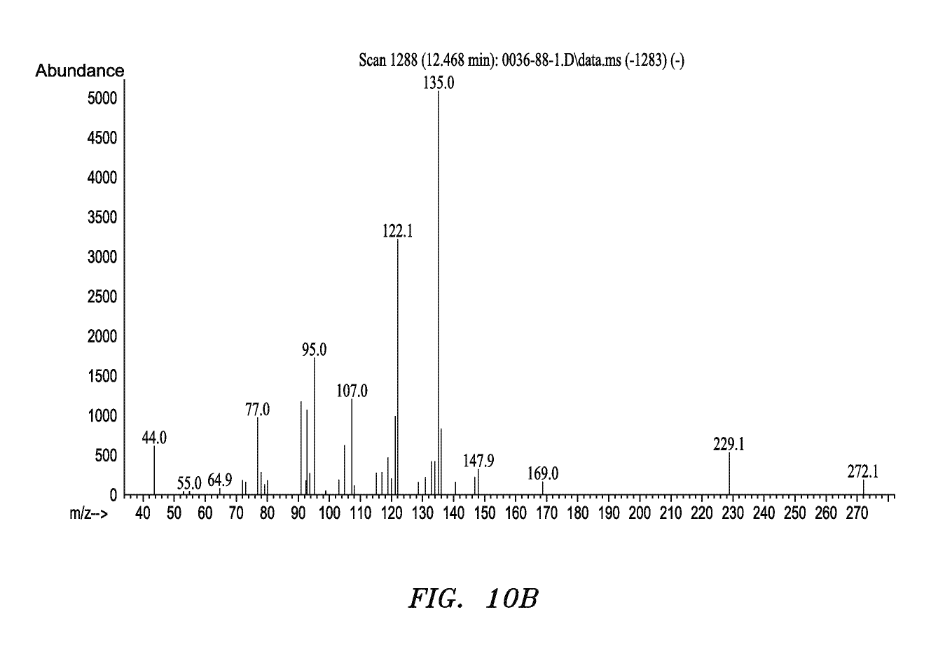

In order to confirm the presence of fusicoccadiene in the gene-positive clones, three of the six clones (clones 1, 3 and 4) were inoculated into liquid medium and grown for 48 hours in the presence of light and 5% CO₂. 3 milliliters of liquid culture of the clones were harvested, pelleted by centrifugation, and resuspended in brine solution. PCC6803 cells expressing a xylanase gene integrated at the same locus (psbY), were utilized as a negative control. Whole cell lysates were then prepared by sonication, and the resulting lysates extracted with 500 ul of heptane for 2 hours at room temperature. After phase separation by centrifugation, the organic layer was analyzed by GC/MSD, Results are shown in FIG. 10A and FIG. 10B.

FIG. 10A shows the m/z=135 extracted ion chromatogram data for three clones (0036-88-1, 0036-88-3, and 0036-88-4 respectively) and a negative control (0036-BD-11). The three fusicoccadiene synthase-containing clones all have a significant peak at 12.48 minutes, while the BD-11 clone does not have a peak. FIG. 10B is the mass spectrometry data for clone number one (0036-88-1) confirming the presence of the fusicoccadiene ions as described in example 4.

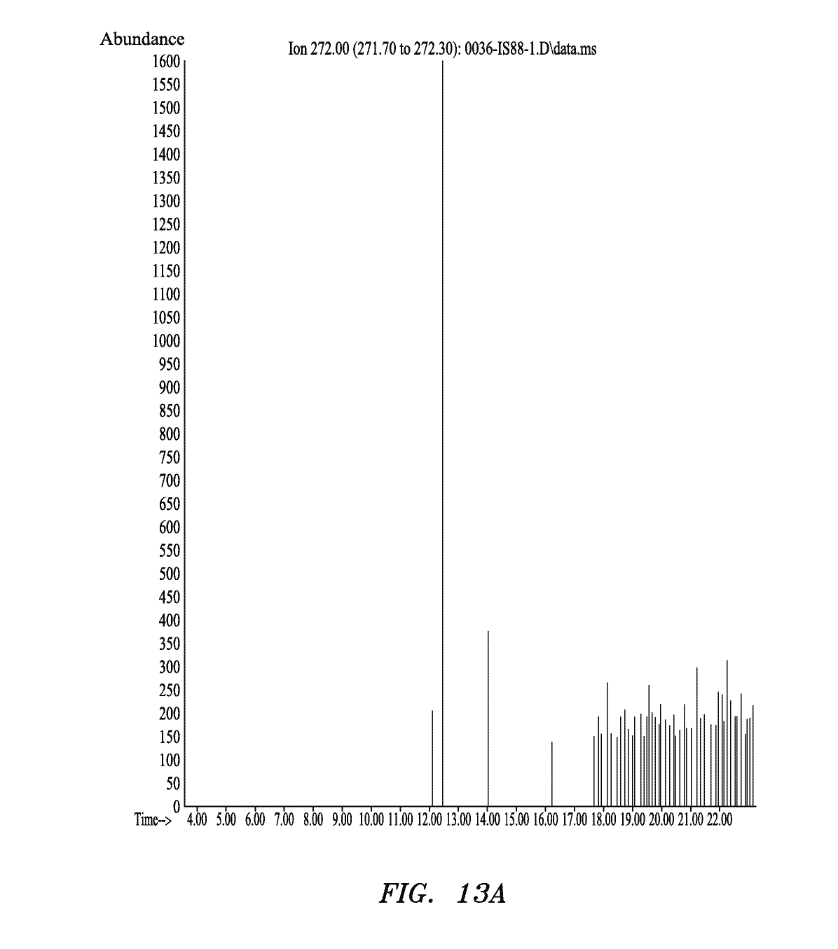

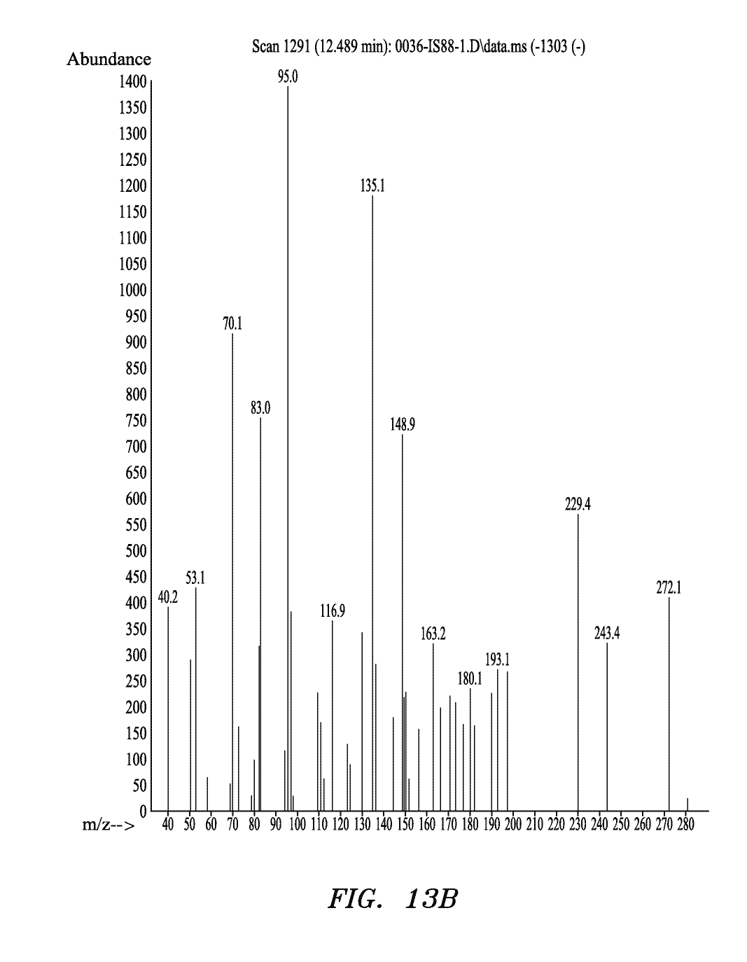

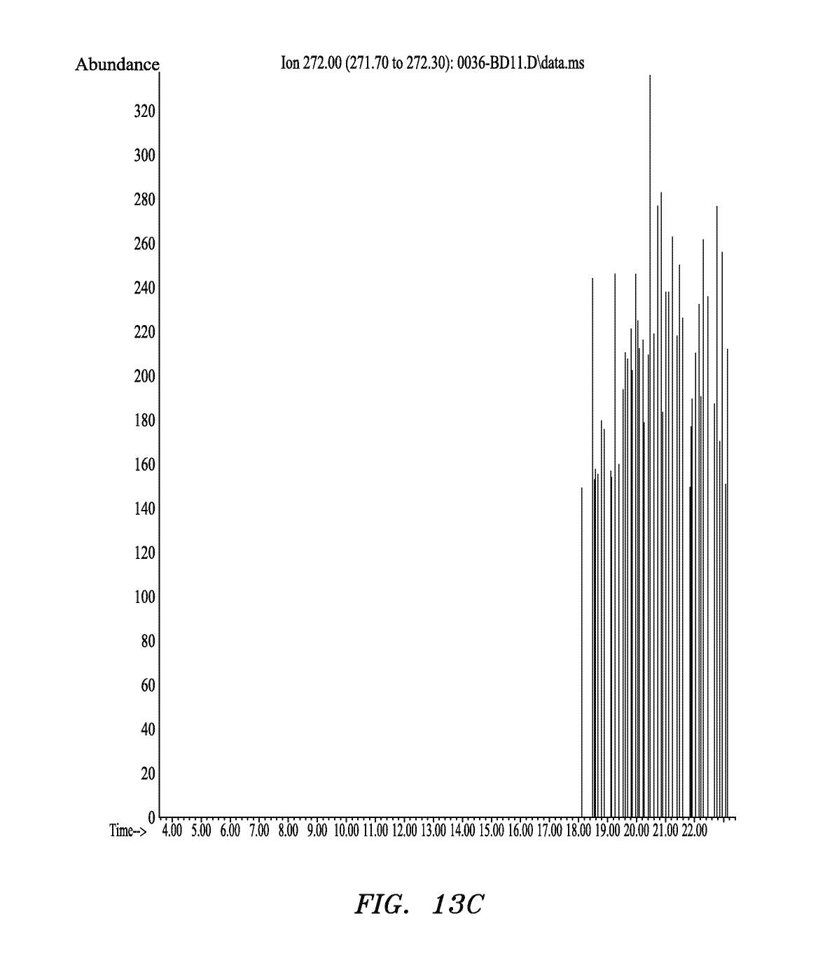

The m/z==272 extracted ion chromatogram and mass spectrum of clone 1 is shown in FIGS. 13A and 13B respectively. The extracted ion chromatogram contains a peak at 12.5 minutes that gives the characteristic mass spectrum for fusicoccadiene containing ions 135, 229 and 272. The m/z=272 extracted ion chromatogram of the negative control containing a xylanase gene instead of PaFs contains no peak at 12.5 minutes (FIG. 13C).

### Example 7

### Expression of the C-Terminal Domain of Fusicoccadiene Synthase

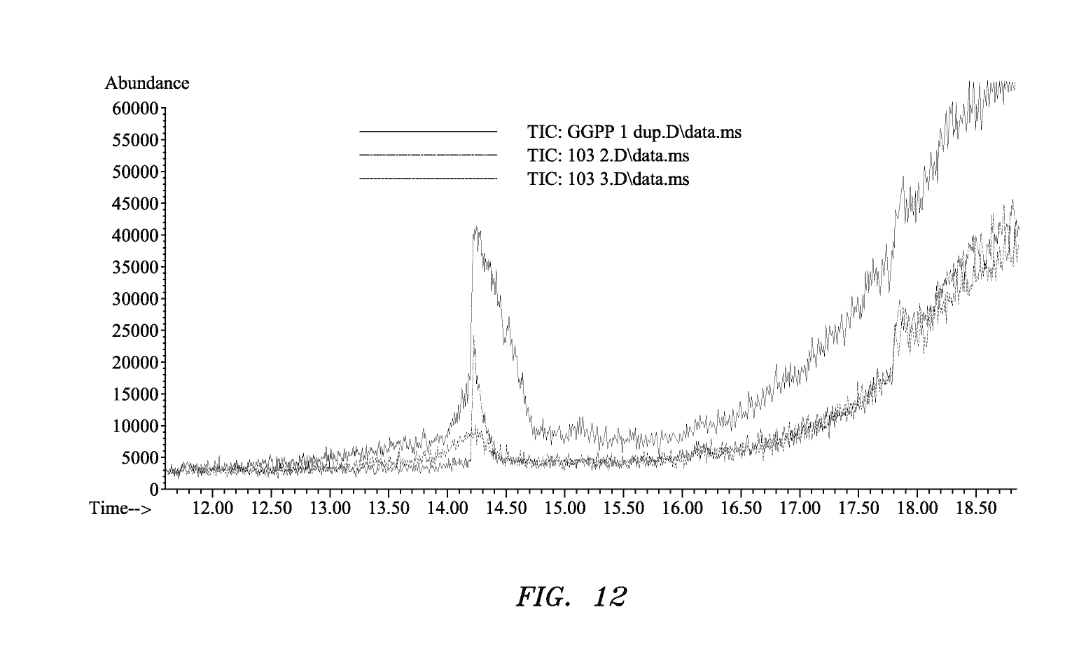

The C-terminal prenyltransferase domain (SEQ ID NO: 40) was cloned into vector pST7 and transformed into E. coli strain BL-2 as described in Example 2. Cells were grown in LB/Kan to an OD₆₀₀ₙₘ=0.6 and induced by the addition of IPTG at 16 C for 24 h. Cells were harvested by centrifugation and the enzyme was purified using streptactin resin [Qiagen, Inc.] as instructed by the manufacturer. The purified enzyme was analyzed by SDS-PAGE to confirm the molecular mass. The purified enzyme was assayed for activity by incubating with IPP and DMAPP, or with IPP and FPP, as substrates. After an overnight incubation at 30 C, the assay mixture was treated with alkaline phosphatase to convert the diphosphate esters into their corresponding alcohols. This mixture was then extracted using heptane, and the heptane extract was analyzed by GC/MSD for the production of geranylgeraniol (GGOH). In addition to the experimental samples, a sample of pure GGPP (Sigma-Aldrich) was treated with phosphatase and extracted as a positive control. A mass spectrum library match confirmed the production of GGOH from both IPP and DMAPP as well as IPP and FPP. Results are shown in FIG. 12.

FIG. 12 shows the total ion chromatograms of three reaction mixture extracts as analyzed by GC/MSD. One sample was of the standard compound, another sample was of the untransformed E. coli cells, and the third sample is of E. coli expressing the GGPP synthase as described above. In this chromatogram, geraniol elutes at time=14.3 minutes. The standard compound GGOH produced a peak with abundance=40000. The sample from untransformed E. coli produced a peak with abundance=7000, and the sample from the GGPP synthase containing E. coli produced a peak with abundance=25000, clearly demonstrating an increase in GGPP production in the transformed bacteria.

### Example 8

### Cloning and Transformation of PaFS Homologs

A GenBank database search for nucleic acids with sequence similarity to PaFS was performed. The nucleotide sequence (SEQ ID NO: 44), encoding the protein EAS27885 (SEQ ID NO: 45) from Coccidioides immitis; the nucleotide sequence (SEQ ID NO: 49) encoding the protein EAA68264 (SEQ ID NO: 50) from Gibberella zeae; and the nucleotide sequence (SEQ ID NO: 54), encoding the protein ACLA_{—}076850 from Aspergillus clavatusi (SEQ ID NO: 55) were found as candidate genes with the potential to contain PaFS-like activity. These genes were synthesized by DNA 2.0 utilizing the most frequent C. reinhardtii codon at each amino acid position except where a change is necessary to eliminate undesired restriction sites (“hot” codon bias). The hot codon optimized nucleic acid encoding protein EAS27885 including the Strep-tag sequence (SEQ ID NO: 47) encodes the protein sequence of SEQ ID NO:48. The hot codon optimized nucleic acid encoding protein EAA68264 including the Strep-tag sequence (SEQ ID NO:52) encodes the protein sequence of SEQ ID NO:53. The hot codon optimized nucleic acid encoding protein ACLA_{—}076850 including the Strep-tag sequence (SEQ ID NO:57) encodes the protein sequence of SEQ ID NO:58. The synthesized genes were cloned into several expression vectors: 1) bacterial expression vector behind the T7 promoter as described in Example 2; 2) Chlamydomonas expression vector behind the tD2 promoter as described in Example 4; 3) Chlamydomonas expression vector behind the D1 promoter as described in Example 4; and 4) Cyanobacterial expression vector behind the tlrtA promoter as described in Example 6. The host cells are cultured in conditions appropriate for bacteria (as described in Example 2), algae (as described in Example 4), or cyanobacteria (as described in Example 6). Cell extracts were prepared and tested for terpenoid production by the GC/MSD described in Example 2.

### Example 9

### Expression of Ent-Kaurene in Algal Host Cells

A gene from Phaeosphaeria nodorum was identified from Genbank (SEQ ID NO: 9) as encoding ent-Kaurene Synthase (SEQ ID NO: 10). A “hot” codon optimized sequence was synthesized by DNA 2.0 (SEQ ID NO: 13) encoding the ent-kaurene synthase with an N-terminal FLAG tag (SEQ ID NO: 14). SEQ ID NO: 13 was cloned into the algal expression vector pSE-3HB-Kan-tD2 and transformed into C. reinhardtii as described in Example 4.

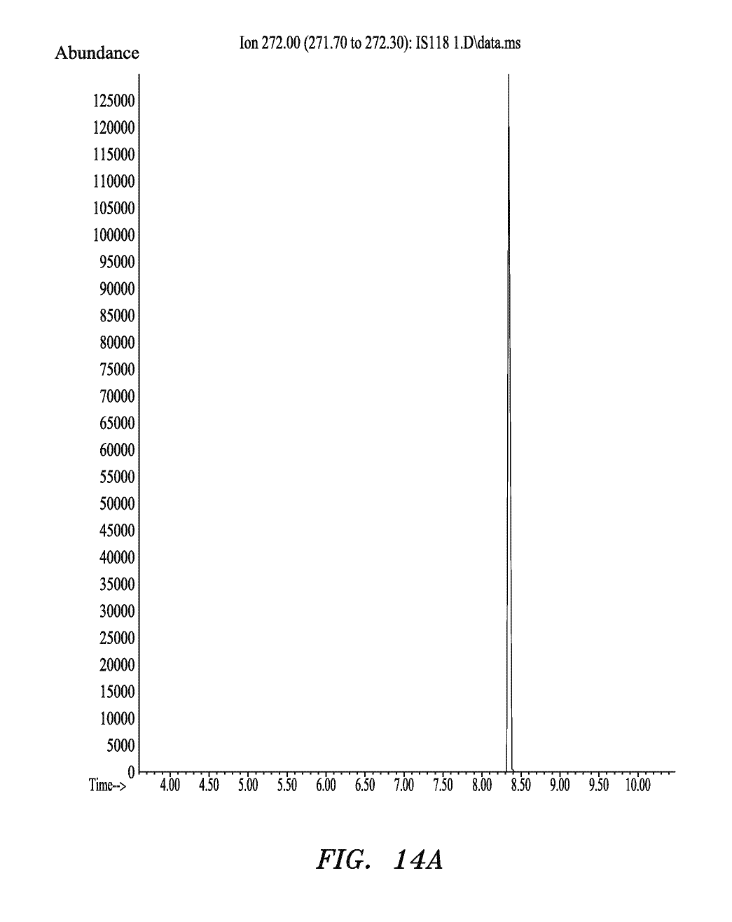

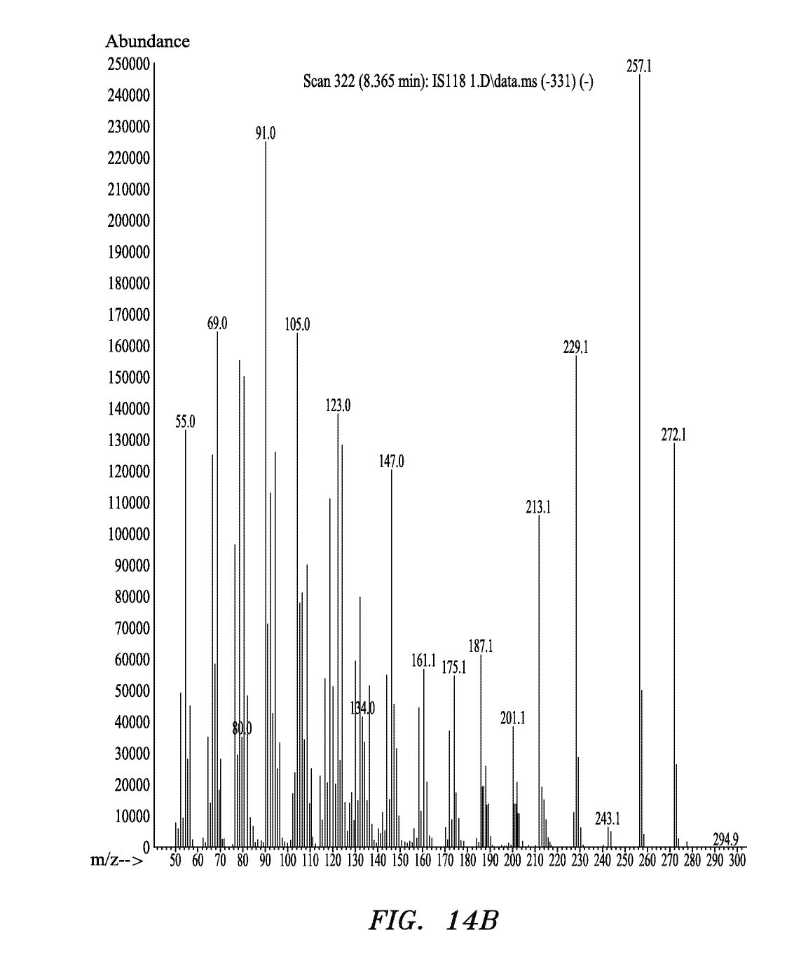

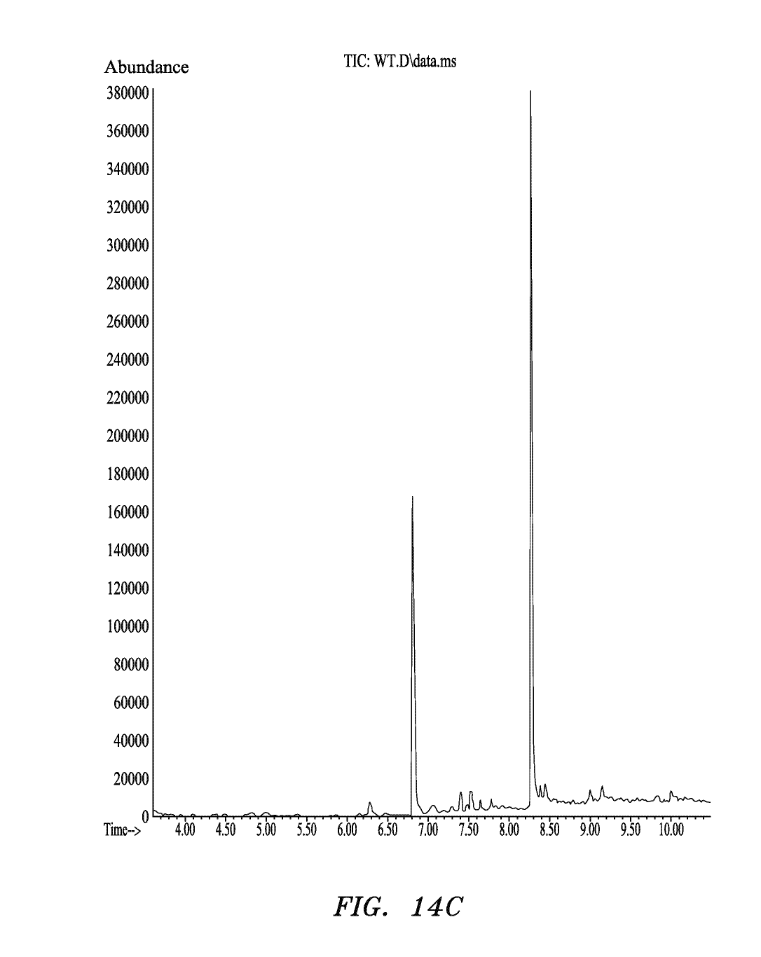

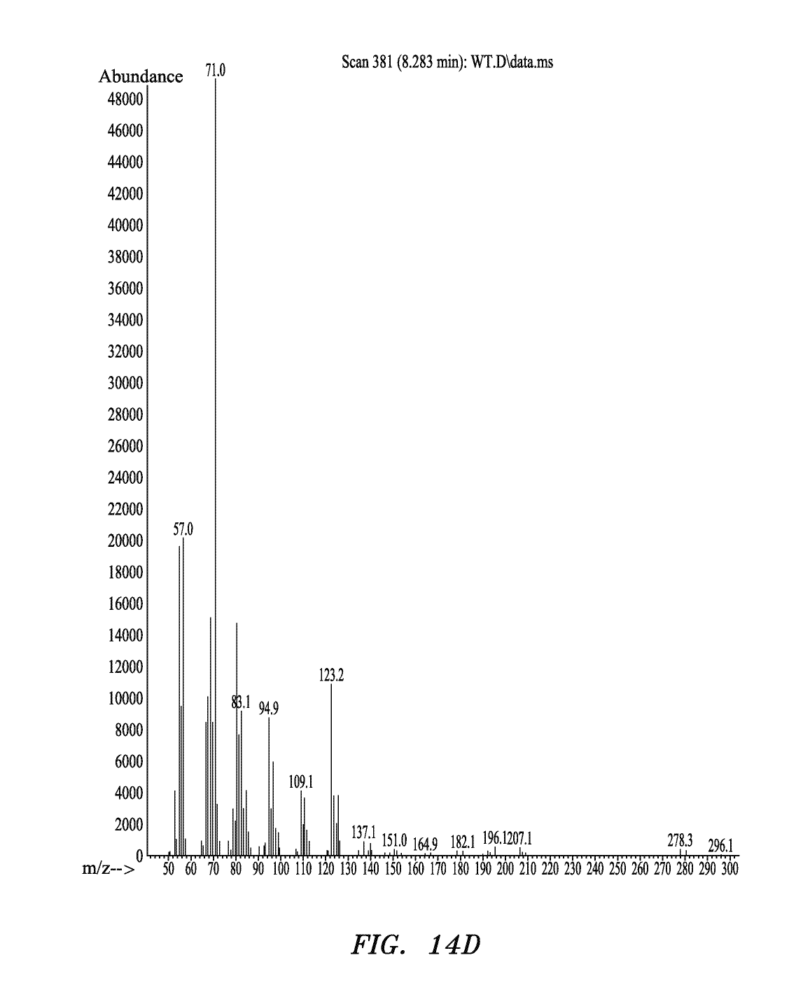

Transformants were grown to mid-log phase and collected by centrifugation and resuspended in brine. Cells were lysed by bead beating with zirconium beads. Whole cell lysates were extracted with 1 mL of heptane by vigorous vortexing. The resulting emulsion was clarified by centrifugation and the heptane was transferred to a glass vial containing a small amount of silica gel. The sample was vortexed and the silica gel allowed to settle. The heptane layer was than analyzed by GC/MSD. FIG. 14A is the m/z=272 extracted ion chromatogram of the organic extract from Chlamydomonas cells expressing ent-kaurene showing a strong peak at 8.36 minutes. The mass spectrum (FIG. 14B) of the peak at 8.36 minutes shows the characteristic ions of ent-kaurene including 229, 257, and 272. Chlamydomonas cells lacking the gene for ent-kaurene were extracted following the same procedure for use as a negative control. The total ion chromatogram of the organic extract of these samples does not contain a peak at 8.36 minutes (FIG. 14C). The mass spectrum of the strong peak at 8.28 minutes does not contain the ions for ent-kaurene namely, 229, 257 and 272 (FIG. 14D).

Ent-kaurene synthase was also cloned and expressed in Scenedesmus cells. The codon optimized ent-Kaurene synthase (SEQ ID NO: 13) was cloned into the Scenedesmus chloroplast expression vector p04-138, which uses the Scenedesmus psbD promoter to drive expression and recombines into the chloroplast genome in an intergenic region near the psbA site. The vector also contains the chloramphenicol acetyl transferase resistance gene driven by the Scenedesmus tufA promoter. Transformants were produced as described in Example 4, except selection was on 25 μg/ml chloramphenicol instead of kanamycin.

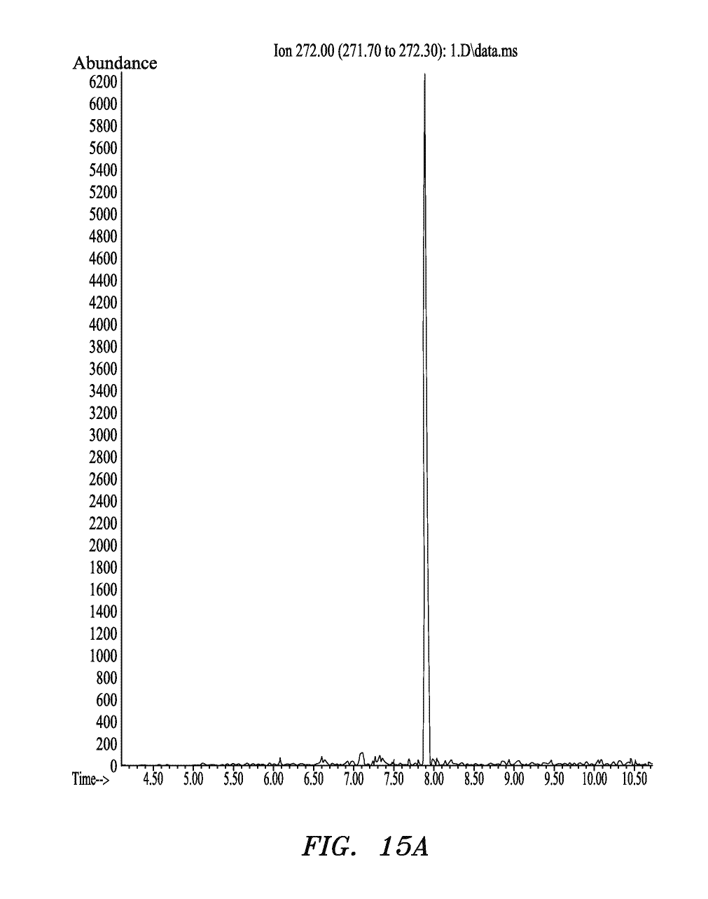

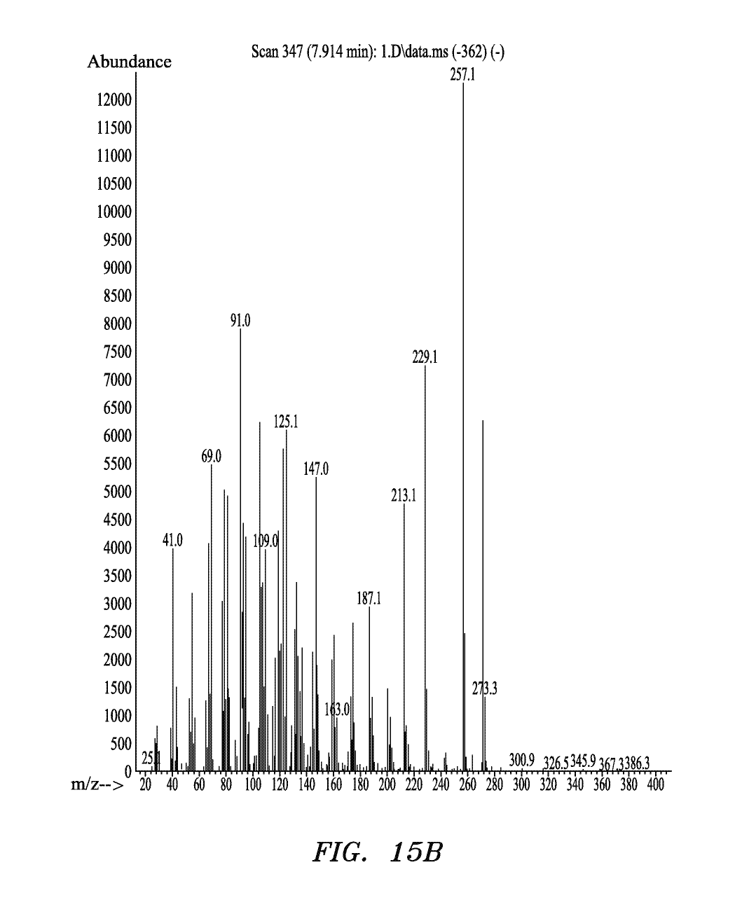

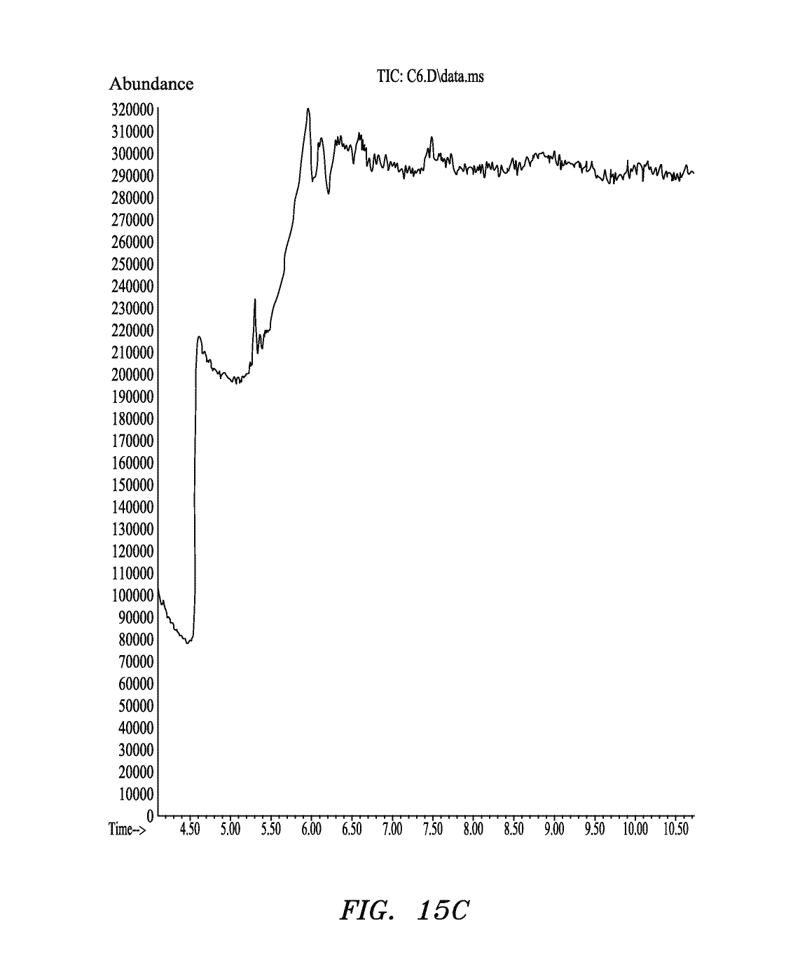

Cells expressing ent-kaurene synthase were lysed and extracted following the same procedure used for the Chlamydomonas samples described in Example 4. The organic extracts of the Scenedesmus samples were analyzed by GC/MSD. FIG. 15A shows the total ion chromatogram for an extract of a Scenedesmus sample that was gene positive for ent-kaurene synthase. The mass spectrum of this peak shown in FIG. 15B contains the molecular ion of 272 as well as the characteristic 229 and 257 ions. Scenedesmus cells which do not contain the ent-kaurene synthase gene were used as a negative control. The total ion chromatogram of the organic extracts from this sample shows no peak at 7.9 minutes (FIG. 15C).

### Example 10

### Expression of Casbene Synthase in Algal Host Cells

A gene from Ricinus communis was identified from Genbank (SEQ ID NO: 15) as encoding Casbene Synthase (SEQ ID NO: 16). A “hot” codon optimized sequence was synthesized by DNA 2.0 (SEQ ID NO: 18) encoding the ent-kaurene synthase with an C-terminal strep tag (SEQ ID NO:20), SEQ ID NO: 18 was cloned into the algal expression vector pSE-3HB-Kan-tD2 and transformed into C. reinhardtii as described in Example 4.

Transformants are grown to mid log phase. Cells are collected by centrifugation and are resuspended in brine. Cells are lysed by bead beating with zirconium beads. Whole cell lysates are extracted with 1 mL of heptane by vigorous vortexing. The resulting emulsion is clarified by centrifugation and the heptane supernatant is transferred to a glass vial containing a small amount of silica gel. The sample is vortexed and the silica gel is allowed to settle. The heptane layer is then analyzed by GC/MSD.

### Example 11

### Synthesis and Expression of Codon-Biased Gene Encoding a Fusion of Casbene Synthase and Geranylgeranyl Diphosphate Synthase

In order to increase the in vivo accumulation of casbene in algae, a gene encoding a fusion of the Ricinus communis casbene synthase and the geranylgeranyl diphosphate synthase domain of Phomopsis amygdali fusicoccadiene synthase was designed using the most frequent C. reinhardtii codon at each amino acid position except where a change was necessary to eliminate undesired restriction sites (“hot” codon bias), and was synthesized by DNA 2.0 (SEQ ID NO: 24), encoding the amino acid sequence SEQ ID NO: 25. In this fusion protein, amino acid residues 1-546 are from the casbene synthase gene, and amino acid residues 547-932 are from the geranyl geranyl diphosphate synthase gene. SEQ ID NO: 24 was cloned into the pSE-3HB-k-tD2 expression vector and transformed into C. reinhardtii as described in Example 4.

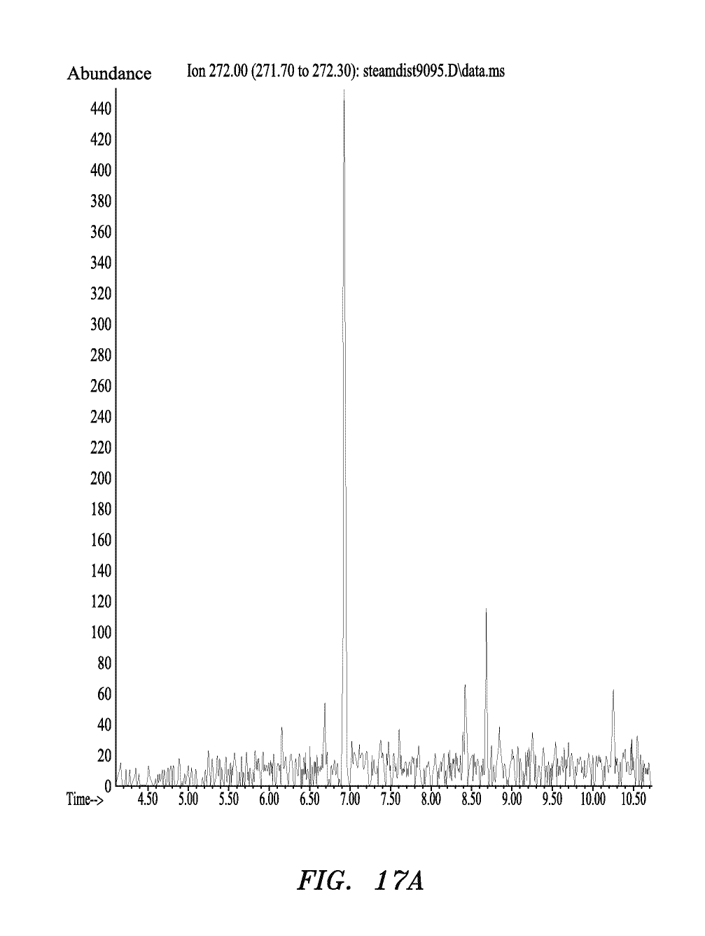

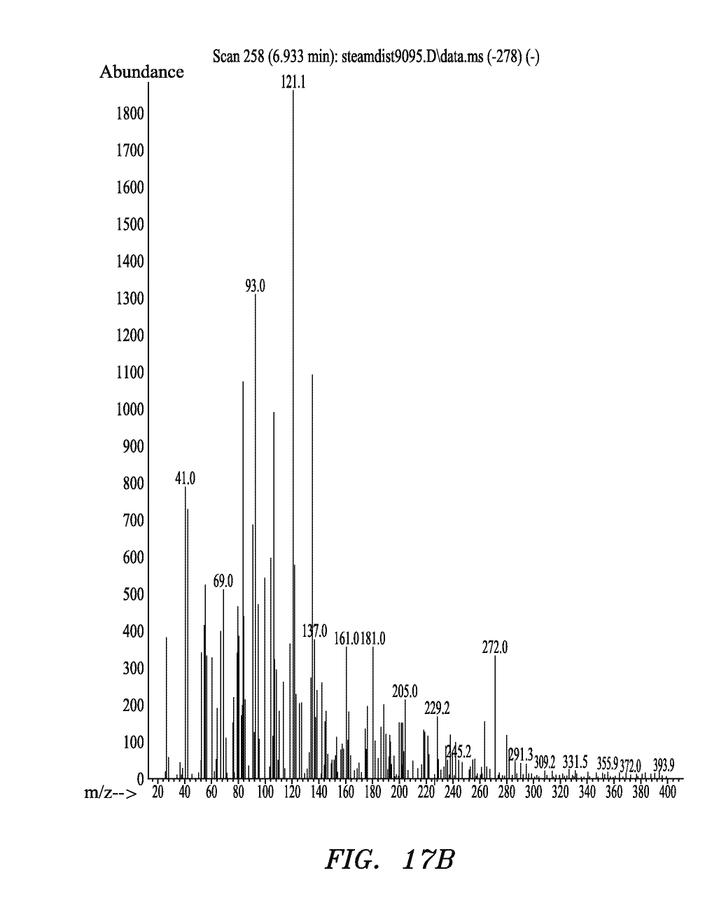

Transformants were grown to produce a 1 L liquid culture. This culture was steam distilled using hexane as the solvent according to the method of H. Maarse and R. Kepner (1970) J. Agric. Food Chem 18(6)1095-1101. After 10 hours at reflux, the hexane fraction was concentrated by rotary evaporation and analyzed by GC/MSD on a FAMEWAX column. FIG. 17A shows the m/z=272 extracted ion chromatogram of the hexane concentrate, showing a peak at 6.93 minutes. FIG. 17B shows the mass spectrum of this peak. The characteristic ions for casbene are present including: 229, 257 and 272. No gene for casbene synthase is present in C. reinhardtii and the wild-type organism does not produce or accumulate casbene.

### Example 12

### Production of Fusicoccadiene in Yeast

The “hot” codon biased PaFS with a Strep tag II (SEQ ID NO: 8) described in Example 1 is cloned into a yeast expression vector pPIC3.5 under the control of the AOX1 promoter, which can be induced by addition of alcohol to the yeast in culture.

To clone the IS-88 gene into the yeast expression vector, the DNA in SEQ ID NO: 8 is amplified by PCR using Primer 1-GGATCCAATAATGGAATTTAAATATTCAGAAG (SEQ ID NO: 42) and Primer 2-GAATTCTTATTTCTCAAATTGAGGGTG (SEQ ID NO: 43). These primers add a BamHI restriction site and Kozak translation initiation site to the 5′ end of the IS-88 gene, and an EcoRI restriction site to the 3′ end of the IS-88 gene. After amplification, both the PCR product and vector pPIC3.5 (Invitrogen, Carlsbad, Calif.) are digested with BamHI and EcoRI; the vector digest is treated with Calf Intestinal Phosphatase, and the digested vector and PCR product are run out on an agarose gel. The gel is stained with ethidium bromide, and the bands corresponding to the digested vector and insert are purified from the gel. The vector and insert are mixed, ligated, and transformed into E. coli. After transformation, the bacteria are plated onto LB solid agar plates containing ampicillin. Resistant colonies are expanded and DNA is prepared from the bacteria, and the vector is again digested with EcoRI and BamHI to confirm the correct insertion of the IS-88 gene.

Once the correct expression vector is isolated, it is introduced into Pichia pastoris according to directions provided with the “Pichia Expression Kit” (Invitrogen, Carlsbad, Calif.). Cultures (2 mls) of Pichia yeast expressing IS-88 are grown and induced using methanol as directed, and collected by centrifugation and resuspended in 250 μls of methanol. Saturated NaC in water (500 μls), 500 μls of petroleum ether, and 250 μs of 1 mm zirconium beads (Bio-spec Products) are added. The solution is vortexed for three minutes and centrifuged at 14,000 g for five minutes at room temperature to separate the organic and aqueous layers. The organic layer (100 μls) is transferred to a vial insert in a standard 2 ml sample vial and analyzed using GC/MSD, as described in Example 2.

### Example 13

### Higher Plant Expression of Fusicoccadiene Synthase

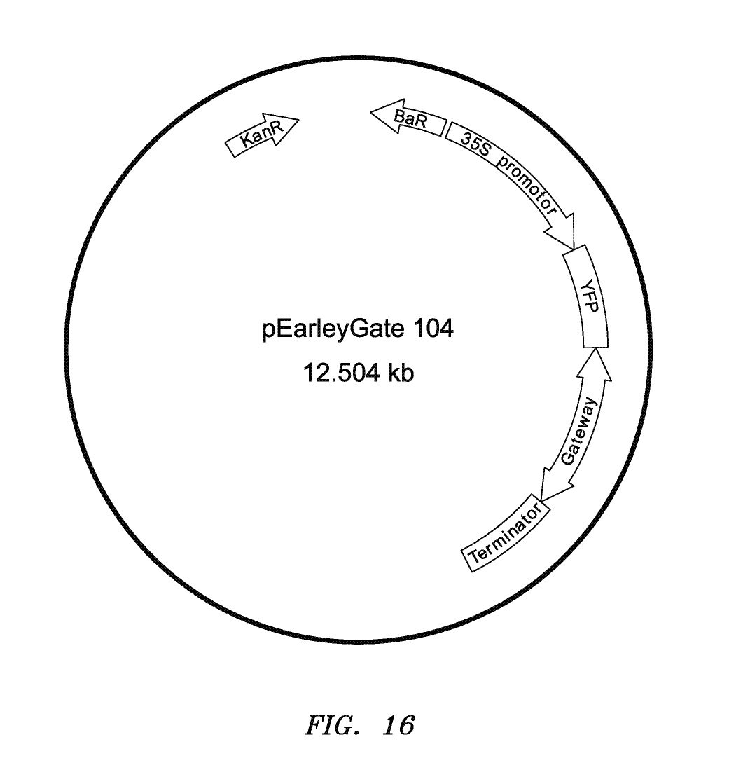

The “hot” codon biased PaFS with a Strep tag II (SEQ ID NO: 8) described in Example I is cloned into a Gateway cloning vector pENTR/D-TOPO (Invitrogen, Carlsbad, Calif.) and then transferred to the plant expression vector pEarleyGate104 (FIG. 16).

To clone the IS-88 gene into the Gateway cloning vector, the DNA in (SEQ ID NO: 8) is amplified by PCR using Primer 1 (CACCATGGAATTTAAATATTCAGAAG (SEQ ID NO: 59) and Primer 2 (TTATTTCTCAAATTGAGGGTG (SEQ ID NO: 60). The primers add a directional topoisomerase cloning sequence to the 5° end of the IS-88 gene. After amplification, the PCR product is mixed with the pENTR/D-TOPO vector and transformed into E. coli. After transformation, the bacteria are plated onto LB solid agar plates containing 50 μg/ml kanamycin. Resistant colonies are grown and DNA is isolated from the cells. The cloning vector containing the IS-88 gene and Gateway recombination sequences is digested with MluI and mixed with pEarleyGate104 DNA and clonase, according to the Invitrogen directions. The reaction mixture is transformed into E. coli and plated onto LB solid agar plates containing 50 μg/ml kanamycin. Resistant colonies are isolated and the plasmid DNA is isolated.

The expression vector pEarleyGate04-1S-88 is introduced into Agrobacterium tumefaciens according to directions provided with the “Agrobacterium transformation kit” (MPBiomedicals Life Sciences, Solon, Ohio). Kanamycin-resistant Agrobacterium cells are isolated on Agrobacterium medium agar (MPBiomedicals Life Sciences, Solon, Ohio) containing kanamycin.

To produce transgenic higher plants, A. tumefaciens bacteria containing the pEarleyGate104-IS88 plasmid are grown in Agrobacterium medium and used to transform Arabidopsis thaliana seedlings according to the method of Clough and Bent (1998, Plant Journal 16:735-743). Transgenic plants are identified by resistance to treatment with the herbicide glufosinate.

Transgenic whole Arabidopsis plants are grown to maturity and ground in a mortar and pestle using 1 ml of methanol per plant. The ground up suspension is transferred to a 2 ml centrifuge tube. Saturated NaCl in water (500 μls), 500 μl of petroleum ether, and 250 μl of mm zirconium beads (Bio-spec Products) are added to the suspension. The solution is vortexed for three minutes and centrifuged at 14,000 g for five minutes at room temperature to separate the organic and aqueous layers. The organic layer (100 μl) is transferred to a vial insert in a standard 2 ml sample vial and analyzed using GC/MSD as in Example 2.

### Example 14

### Use of a Diterpene Synthase as a Readout of Isoprenoid Pathway Metabolic Flux

Algal cells expressing the “I-lot” codon optimized fusicoccadiene synthase (SEQ ID NO:8) are cultured in a number of different conditions expected to modulate the flux through the isoprenoid pathway. These conditions include reduction of nitrogen levels in the growth media, reduction of sulfur levels in the growth media, reduction or increase in light levels during growth, and modulation of temperature during growth, among others. Cells are collected by centrifugation and extracted with organic solvent as described in Example 2. The organic extracts are analyzed by GC/MSD to quantify the relative amount of fusicoccadiene present in the algae, and normalized to either the number of cells per volume or the ash-free dry weight per volume of the test cultures. The relative amount of fusicoccadiene present reflects the flux through the isoprenoid pathway under the different culture conditions.

In the same manner, genetic induction of changes in flux through the isoprenoid pathway can be determined by quantifying fusicoccadiene levels. Algae expressing fusicoccadiene synthase are modified genetically by a number of means, including mutagenesis, breeding, introduction of other transgenes, or gene silencing using recombinant nucleic acids (for example, siRNA or miRNA). The quantity of fusicoccadiene present is measured as above. The relative amount of fusicoccadiene present again reflects the flux through the isoprenoid pathway.

Technical and scientific terms used herein have the meanings commonly understood by one of ordinary skill in the art to which the instant disclosure pertains, unless otherwise defined. Reference is made herein to various materials and methodologies known to those of skill in the art. Standard reference works setting forth the general principles of recombinant DNA technology include, for example, Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of yeast genetics useful for selected aspects of the disclosure include: Sherman et al. “Laboratory Course Manual Methods in Yeast Genetics”, Cold Spring Harbor Laboratory, Cold Spring Harbor, N.Y., 1986, and Guthrie et al., “Guide to Yeast Genetics and Molecular Biology”, Academic, New York, 1991.

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A non-vascular photosynthetic organism comprising a nucleic acid encoding a protein comprising (a) an amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55; or (b) an amino acid sequence of at least 90% identity to SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55.

2. The organism of claim 1, wherein said nucleic acid encodes a protein comprising the amino acid sequence of SEQ ID No. 2 or an amino acid sequence of at least 90% identity to SEQ ID NO. 2.

3. The organism of claim 2 wherein said nucleic acid comprises SEQ ID NO. 1, SEQ ID NO. 4, SEQ ID NO, 5, SEQ ID NO. 7 or SEQ ID NO, 8.

4. The organism of claim 1, wherein said nucleic acid encodes a protein comprising the amino acid sequence of SEQ ID No. 38 or an amino acid sequence of at least 90% identity to SEQ ID NO. 38.

5. The organism of claim 4 wherein said nucleic acid comprises SEQ ID NO. 37, SEQ ID NO. 39 or SEQ ID NO. 40.

6. The organism of claim 1, wherein said nucleic acid encodes a protein comprising the amino acid sequence of SEQ ID No. 10 or an amino acid sequence of at least 90% identity to SEQ ID NO. 10.

7. The organism of claim 6 wherein said nucleic acid comprises SEQ ID NO. 9, SEQ ID NO. 11 or SEQ ID NO. 13.

8. The organism of claim 1, wherein said nucleic acid encodes a protein comprising the amino acid sequence of SEQ ID No. 16 or an amino acid sequence of at least 90% identity to SEQ ID NO. 16.

9. The organism of claim 8 wherein said nucleic acid comprises SEQ ID NO. 15, SEQ ID NO. 17 or SEQ ID NO. 18.

10. The organism of claim 1, wherein said nucleic acid encodes a protein comprising the amino acid sequence of SEQ ID No. 22 or an amino acid sequence of at least 90% identity to SEQ ID NO. 22.

11. The organism of claim 10 wherein said nucleic acid comprises SEQ ID NO. 21, or SEQ ID NO. 24.

12. The organism of claim 1, wherein said nucleic acid encodes a protein comprising the amino acid sequence of SEQ ID No. 27 or an amino acid sequence of at least 90% identity to SEQ ID NO. 27.

13. The organism of claim 12 wherein said nucleic acid comprises SEQ ID NO. 26, SEQ ID NO. 28 or SEQ ID NO. 30.

14. The organism of claim 1, wherein said nucleic acid encodes a protein comprising the amino acid sequence of SEQ ID No. 33 or an amino acid sequence of at least 90% identity to SEQ ID NO. 33.

15. The organism of claim 14 wherein said nucleic acid comprises SEQ ID NO. 32, SEQ ID NO. 34 or SEQ ID NO. 35.

16. The organism of claim 1, wherein said nucleic acid encodes a protein comprising the amino acid sequence of SEQ ID No. 45, SEQ ID NO. 50 or SEQ ID NO. 55 or an amino acid sequence of at least 90% identity to SEQ ID No. 45, SEQ ID NO. 50 or SEQ ID NO. 55.

17. The organism of claim 16, wherein said nucleic acid sequence comprises SEQ ID NO. 44, SEQ ID NO. 46, SEQ ID NO. 47, SEQ ID NO. 49, SEQ ID NO. 51, SEQ ID NO. 52, SEQ ID NO. 54, SEQ ID NO. 56 or SEQ ID NO. 57.

18. A method of producing a terpenoid or terpene in a non-vascular photosynthetic organism, comprising transforming a non-vascular photosynthetic organism with a nucleic acid encoding a protein comprising (a) an amino acid sequence of SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55; or (b) an amino acid sequence of at least 90% identity to SEQ ID NO: 2, SEQ ID NO: 10, SEQ ID NO: 16, SEQ ID NO: 22, SEQ ID NO: 27, SEQ ID NO: 33, SEQ ID NO: 38, SEQ ID NO: 45, SEQ ID NO: 50, or SEQ ID NO: 55; and expressing said nucleic acid in said organism.

19. The method of claim 18, wherein said nucleic acid encodes a protein comprising the amino acid sequence of SEQ ID No. 2, SEQ ID NO. 38, or an amino acid sequence of at least 90% identity to SEQ ID NO. 2 or SEQ ID NO. 38.

20. The method of claim 19, wherein said nucleic acid comprises SEQ ID NO. 1, SEQ ID NO. 4, SEQ ID NO. 5, SEQ ID NO. 7, SEQ ID NO. 8, SEQ ID NO. 37, SEQ ID NO. 39 or SEQ ID NO.

21. The method of claim 18, wherein said nucleic acid encodes a protein comprising the amino acid sequence of SEQ ID No. 10 or an amino acid sequence of at least 90% identity to SEQ ID NO. 10.

22. The method of claim 21, wherein said nucleic acid comprises SEQ ID NO. 9, SEQ ID NO. 11 or SEQ ID NO. 13.

23. The method of claim 18, wherein said nucleic acid encodes a protein comprising the amino acid sequence of SEQ ID No. 16, SEQ ID NO. 22, or an amino acid sequence of at least 90% identity to SEQ ID NO. 16 or SEQ ID NO. 22.

24. The method of claim 18, wherein said nucleic acid encodes a protein comprising the amino acid sequence of SEQ ID No. 27 or an amino acid sequence of at least 90% identity to SEQ ID NO. 27.

25. The method of claim 24 wherein said nucleic acid comprises SEQ ID NO. 26, SEQ ID NO. 28 or SEQ ID NO. 30.

26. The method of claim 18, wherein said nucleic acid encodes a protein comprising the amino acid sequence of SEQ ID No, 33 or an amino acid sequence of at least 90% identity to SEQ ID NO. 33.

27. The method of claim 14, wherein said nucleic acid comprises SEQ ID NO. 32, SEQ ID NO. 34 or SEQ ID NO. 35.

28. The method of claim 18, wherein said nucleic acid encodes a protein comprising the amino acid sequence of SEQ ID No. 45, SEQ ID NO. 50 or SEQ ID NO. 55 or an amino acid sequence of at least 90% identity to SEQ ID No. 45, SEQ ID NO. 50 or SEQ ID NO. 55.

29. The organism of claim 28, wherein said nucleic acid sequence comprises SEQ ID NO. 44, SEQ ID NO. 46, SEQ ID NO. 47, SEQ ID NO. 49, SEQ ID NO. 51, SEQ ID NO. 52, SEQ ID NO. 54, SEQ ID NO. 56 or SEQ ID NO. 57.
